(19) United States
(12) Patent Application Publication
(10) Pub. No.: US 2018/0325804 A1
(43) Pub. Date: Nov. 15, 2018

(54) TOPICAL MUSCADINE FORMULATION FOR COSMETIC USE

(71) Applicants: Sonhee C. Park, Pleasanton, CA (US); Christos Kyrou, (US); Teodoro Ianiro, (US)

(72) Inventors: Sonhee C. Park, Pleasanton, CA (US); Christos Kyrou, Goshen, NY (US); Teodoro Ianiro, Concord, CA (US)

(73) Assignee: Shaklee Corporation, Pleasanton, CA (US)

(21) Appl. No.: 15/976,588

(22) Filed: May 10, 2018

Related U.S. Application Data
(60) Provisional application No. 62/505,549, filed on May 12, 2017.

(51) Int. Cl. A61K 8/9789; A61K 8/73; A61Q 19/00; A61Q 19/10; A61K 8/67; A61K 8/66; A61Q 19/08

(52) U.S. Cl. CPC A61K 8/9789; A61K 8/73; A61Q 19/00; A61Q 19/10; A61K 8/673; A61K 2800/88; A61K 8/676; A61K 8/678; A61K 8/66; A61Q 19/08; A61K 8/671

(57) ABSTRACT

Skin care regimens are disclosed, which use five components that each include a composition made with a combination of a decolorized muscadine (Vitis rotundifolia) pomace solvent extract, beta-glucan and grape seed extract, to improve the appearance of skin. The five components include a toner, day moisturizer, night moisturizer, cleanser and serum. Also disclosed are skin care methods to improve the appearance of skin by the application of effective amounts of these five components.

### CROSS REFERENCE TO RELATED APPLICATION(S)

This claims the benefit of U.S. Provisional Application No. 62/505,549, filed May 12, 2017, which is incorporated herein by reference.

### FIELD

This disclosure relates to combinations for the treatment of the skin that include components: a toner, a day moisturizer, a night moisturizer, a cleanser and/or a serum. Each of the components includes a decolorized muscadine (Vitis rotundifolia) pomace solvent extract, beta-glucan and a grape seed extract. Also disclosed are methods for improving the appearance of the skin using these combinations.

### BACKGROUND

Reactive oxygen species (ROS) are byproducts of aerobic metabolism and thus are generated continuously in humans and other organisms. Humans are also exposed to ROS from environmental sources such as pollution, sunlight and diet. While there are different chemical forms of ROS, they all produce deleterious actions on the structure and function of cellular constituents and macromolecules, including in the skin. The process of skin aging is complex, involving both chronological and environmental factors. The outermost layer of the skin, the epidermis, provides a barrier against chemical and biological insults. One function of the epidermis is to moderate the penetration of ROS into deeper skin layers.

Over time, the rate of generating new layers of skin in the epidermis slows, causing the skin to gradually thin. This can reduce the ability of the epidermis to provide adequate protection from harmful stimuli, such as damaging environmental factors. A weakened barrier can lead to, for example, a degradation of collagen and elastin, which can be manifested as skin wrinkling, and an increase in permeability to ROS. The capacity of the epidermis to recover from harmful stimuli is also reduced with age.

Grape extracts are known to have beneficial anti-oxidant properties. Muscadine grapes, for example, contain several bioactive polyphenolic compounds, including flavonoids (such as flavonols, anthocyanins, and flavanones, as well as flavan-3-ols and oligomers thereof known as proanthocyanidins) and non-flavonoids (such as phenolic acids, tannins and stilbene derivatives, for example resveratrol). The biological effects of flavonoids are believed to be due to free radical scavenging, beneficial effects on cellular signaling pathways and gene expression, and selective interference with the cell division cycle of rapidly and abnormally proliferating mammalian cells.

U.S. Pat. No. 4,272,544 discloses the application and use of four skin care products; a skin cleanser formulation, a skin tonic, a skin cream, and a skin lotion formulation. U.S. Published Patent Application No. 2004/0191330 discloses a skin care regimen including a hydrating wash, a toner-astringent, an eye cream, a facial serum having alpha hydroxy and beta hydroxy acids, a day cream having a sunscreen agent, and a night cream having spent grain wax and Glycine Soja (Soybean) Protein. The use of multiple components in a skin care regimen can enhance production of collagen and elastin, and reduce free radicals. However, a need remains for additional skin care regimens that can reduce fine lines and wrinkles, and improve the appearance of the skin.

### SUMMARY

Methods and compositions for improving the appearance of skin are disclosed herein, incorporating decolorized grape pomace solvent extracts. Use of compositions that include a decolorized grape pomace solvent extract, beta-glucan and grape seed extract can improve the appearance of skin by, for example, reducing the depth of wrinkles, reducing the number of wrinkles, reducing the length of wrinkles, reducing the width of wrinkles, reducing skin roughness, increasing skin smoothness, increasing skin radiance, increasing skin firmness, reducing skin sagging, increasing the evenness of skin tone, reducing pore size, reducing skin hyperpigmentation, or any combination of these improvements. Skin care methods are disclosed which use a combination of components. The rate of application can be, but is not limited to, once or twice a day for one or multiple weeks. In some embodiments, a combination is used that includes including five components: a toner, a day moisturizer, a night moisturizer, a cleanser and a serum.

Advances in the production of decolorized grape solvent extracts allow for the use of such grape extracts in topical preparations, including for skin care products. The present disclosure provides compositions that include a decolorized grape pomace extract that has a lower condensed tannin content while substantially preserving polyphenols and desirable anti-oxidant activity in the extract. In some examples, the decolorized extract is obtained from bronze and/or purple muscadine (Vitis rotundifolia) grape pomace. The presently disclosed compositions also include grape seed extract and beta-glucan.

In certain examples, the compositions include an effective amount of decolorized muscadine (Vitis rotundifolia) pomace solvent extract, beta-glucan and grape seed extract, wherein the decolorized muscadine pomace solvent extract comprises a liquid bronze muscadine pomace extract combined with a liquid purple muscadine pomace extract to produce a liquid muscadine pomace extract, wherein a) the bronze muscadine pomace extract and the purple muscadine pomace extract are solvent extracts, b) the mixture of bronze muscadine pomace extract and purple muscadine pomace extract promotes solubility of ellagic acid in the muscadine pomace extract, c) the bronze muscadine pomace extract and the purple muscadine pomace extract are filtered and fermented extracts, and d) the muscadine pomace extract has a polyphenol content of at least about 2%. Also disclosed is combinations of these components, for use in improving the appearance of skin. Thus, methods are disclosed for improving the appearance of the skin, such as the facial skin.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph showing the percentage of human subjects exhibiting an improvement in under eye skin health after 12 weeks of following an exemplary skin care regimen, in reference to a reduction in wrinkle number, length, width, area and depth.

FIG. 2 is a graph showing the magnitude of improvement in under eye skin health in the subjects of FIG. 1.

FIG. 3 is a graph showing the percentage of human subjects exhibiting an improvement in crow's feet skin health after 12 weeks of following an exemplary skin care regimen, in reference to a reduction in wrinkle number, length, width, area and depth.

FIG. 4 is a graph showing the magnitude of improvement in crow's feet skin health in the subjects of FIG. 3.

FIG. 5 is a graph showing the percentage of human subjects exhibiting an improvement in skin roughness after 12 weeks of following an exemplary skin care regimen, in reference to skin roughness depth, maximum and average roughness, and skin smoothness.

FIG. 6 is a graph showing the magnitude of improvement in skin roughness in the subjects of FIG. 5.

FIG. 7 is a graph showing the percentage of subjects exhibiting an improvement in skin firmness after 8 weeks of following an exemplary skin care regimen (left bar), and the magnitude of improvement in skin firmness (right bar).

FIG. 8 shows two graphs, one of the percentage of subjects exhibiting an improvement in skin health as measured by an analysis of global facial wrinkles after 1, 2, 4, 8 and 12 weeks of following an exemplary skin care regimen (left graph), and the other of the corresponding average percentage of improvement in skin health (right graph).

FIG. 9 shows two graphs, one of the percentage of subjects exhibiting an improvement in skin health as measured by an analysis of periorbital wrinkles after 1, 2, 4, 8 and 12 weeks of following an exemplary skin care regimen (left graph), and the other of the corresponding average percentage of improvement in skin health (right graph).

FIG. 10 shows two graphs, one of the percentage of subjects exhibiting an improvement in skin health as measured by an analysis of forehead wrinkles after 1, 2, 4, 8 and 12 weeks of following an exemplary skin care regimen (left graph), and the other of the corresponding average percentage of improvement in skin health (right graph).

FIG. 11 shows two graphs, one of the percentage of subjects exhibiting an improvement in skin health as measured by an analysis of cheek wrinkles after 1, 2, 4, 8 and 12 weeks of following an exemplary skin care regimen (left graph), and the other of the corresponding average percentage of improvement in skin health (right graph).

FIG. 12 shows two graphs, one of the percentage of subjects exhibiting an improvement as measured by an analysis of overall skin health after 1, 2, 4, 8 and 12 weeks of following an exemplary skin care regimen (left graph), and the other of the corresponding average percentage of improvement in skin health (right graph), as determined by visual grading by an expert skin grader.

FIG. 13 shows two graphs, one of the percentage of subjects exhibiting an improvement in skin health as measured by an analysis of skin texture after 1, 2, 4, 8 and 12 weeks of following an exemplary skin care regimen (left graph), and the other of the corresponding average percentage of improvement in skin health (right graph).

FIG. 14 shows two graphs, one of the percentage of subjects exhibiting an improvement in skin health as measured by an analysis of skin radiance after 1, 2, 4, 8 and 12 weeks of following an exemplary skin care regimen (left graph), and the other of the corresponding average percentage of improvement in skin health (right graph).

FIG. 15 shows two graphs, one of the percentage of subjects exhibiting an improvement in skin health as measured by an analysis of skin firmness after 1, 2, 4, 8 and 12 weeks of following an exemplary skin care regimen (left graph), and the other of the corresponding average percentage of improvement in skin health (right graph).

FIG. 16 shows two graphs, one of the percentage of subjects exhibiting an improvement in skin health as measured by an analysis of skin sagging after 1, 2, 4, 8 and 12 weeks of following an exemplary skin care regimen (left graph), and the other of the corresponding average percentage of improvement in skin health (right graph).

FIG. 17 shows two graphs, one of the percentage of subjects exhibiting an improvement in skin health as measured by an analysis of skin tone evenness after 1, 2, 4, 8 and 12 weeks of following an exemplary skin care regimen (left graph), and the other of the corresponding average percentage of improvement in skin health (right graph).

FIG. 18 shows two graphs, one of the percentage of subjects exhibiting an improvement in skin health as measured by an analysis of global facial pore size after 1, 2, 4, 8 and 12 weeks of following an exemplary skin care regimen (left graph), and the other of the corresponding average percentage of improvement in skin health (right graph).

FIG. 19 shows two graphs, one of the percentage of subjects exhibiting an improvement in skin health as measured by an analysis of global facial skin hyperpigmentation after 1, 2, 4, 8 and 12 weeks of following an exemplary skin care regimen (left graph), and the other of the corresponding average percentage of improvement in skin health (right graph). Skin that exhibited darker spots or mottled spots on the face, indicating more intense pigmentation in those areas, was considered to be hyperpigmented.

FIG. 20 is a graph showing the percentage of subjects that perceived an improvement in their skin health in 13 specific parameters after 12 weeks of following an exemplary skin care regimen.

FIG. 21 is a flow chart of an exemplary extraction method that lowers the condensed tannin content while substantially preserving polyphenol content, in an embodiment of a process used to produce a decolorized muscadine pomace solvent extract.

FIG. 22 is schematic depiction of a system for carrying out the method of FIG. 21.

FIGS. 23A-23B are comparative trace of chromatographic profiles of polyphenols in a precursor muscadine pomace extract before (23A) and the decolorized extract after (23B) the tannin content is lowered using an embodiment of a process used to produce a decolorized muscadine pomace solvent extract. The desired polyphenolic profile of the precursor extract is retained, while the level of condensed tannins is greatly lowered in the decolorized extract.

FIG. 24 is a graph illustrating the results of the elastase inhibition test with the decolorized muscadine pomace solvent extract, demonstrating an effect on skin elasticity by inhibiting elastin reduction.

FIG. 25 is a graph illustrating the results of a collagenase inhibition test with the decolorized muscadine pomace solvent extract. Enhanced collagenase inhibition improves skin firmness by avoiding collagen loss in the skin.

FIGS. 26A-26B show the results of a DPPH assay that measures free radical scavenging power as shown in Trolox equivalents (TE) with the decolorized muscadine pomace solvent extract; the higher the TE value the greater the anti-oxidant power. As shown in FIG. 26B, 100 mcg/ml of TME showed the same antioxidant power as 185 mcg of Trolox indicating that TME is 1.85 times more potent antioxidant than Trolox as measured by DPPH assay. Trolox is a known powerful antioxidant used as a control substance for antioxidant tests.

FIGS. 27A-27B show the results of a TT Dimer Assay. FIG. 27A shows that 1% decolorized muscadine pomace solvent extract pretreatment before ultraviolet B (UVB) exposure completely prevented DNA damage. The extract was better than the positive control (1 mM Trolox). A 1% decolorized muscadine pomace solvent extract treatment after UVB exposure (post-treatment) also showed 45% reduction in DNA damage, suggesting an effect on DNA repair; the effect from 0.1% was lower than 1% in both pre- and post-treatments. FIG. 27B presents additional results. TME at 0.25% showed a significant reduction (38%) in TT Dimer formation as compared to the untreated control. GSE+glucan did not show significant reductions as compared to the control. When GSE+glucan were combined with TME, the reduction in TT-dimer formation were more significant (69% reduction and 44% reduction), suggesting a combined effect of these the ingredients, all of which are included in the components of the disclosed combinations.

FIG. 28 shows cell survival after UVB exposure; the decolorized muscadine pomace solvent extracts significantly increased cell survival as compared to untreated cells, and the increase was larger than the increase seen with 20 μM Trolox, a positive control treatment.

FIG. 29 shows tyrosinase inhibition at different concentrations of the decolorized muscadine pomace solvent extract, indicating an ability to diminish hyperpigmentation.

FIG. 30 is a graph of a TT Dimer DNA damage comparison assay between a decolorized topical muscadine pomace solvent extract (TME) and orally administered muscadine pomace extract (OME) that has not been decolorized; there was no statistical difference in reducing DNA damage between OME and TME containing 0.09% and 0.009% of polyphenols. The OME is the extract disclosed in prior U.S. Pat. No. 8,568,804, U.S. Pat. No. 9,132,162 and U.S. Pat. No. 9,173,916.

FIG. 31 is a graph illustrating that both the OME (not decolorized, ●) and TME (decolorized, ▪) demonstrated excellent activity (efficacy) in inhibiting Advanced Glycation End products (AGE) formation in a dose-related. Manner maximal inhibition of AGE formation reached 95-100% at concentrations of 15-20 μg polyphenols/ml for both extracts. However, the OME was more potent than the TME as indicated by the concentrations required to inhibit AGE formation by 50% (IC₅₀) The IC₅₀ value for the OME was 0.65 μg polyphenols/ml whereas the IC₅₀ value for the TME was 3.94 μg polyphenols/ml. This suggests that while tannins contribute to the AGE-inhibitory activity, the other muscadine polyphenols remaining in the TME are equally efficacious in inhibiting protein glycation as those found in the OME.

### DETAILED DESCRIPTION

Disclosed herein are compositions containing an effective amount of decolorized muscadine (Vitis rotundifolia) pomace solvent extract, beta-glucan and grape seed extract, and their use in combinations for improving the appearance of the skin. Additionally disclosed are methods of skin care using these compositions to improve the appearance of skin. The compositions may additionally include other ingredients that are present in a sufficient amount to improve the appearance of skin, such as panthenol, Vitamins A, C, and E, superoxide dismutase, schizandra chinensis extract, lotus japonicus extract, or any combination thereof. An improvement in the appearance of skin may be measured by a reduction in the depth, length, width, or number of wrinkles; a reduction in skin roughness, skin sagging, pore size, or skin hyperpigmentation; and/or an increase in skin smoothness, radiance, firmness, or the evenness of skin tone.

### I. TERMS

The following explanations of terms and methods are provided to better describe the present disclosure and to guide those of ordinary skill in the art in the practice of the present disclosure. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. All percentages and ratios are calculated by weight unless otherwise indicated. The term “about” refers to an amount within a specific range of a value. For example, “about” a specific molecular weight or gram amount indicates within 5% of that molecular weight or gram amount. In a non-limiting example, “about” 100 grams refers to 95 grams to 105 grams. In addition, “about” a specific percentage refers to within 0.05%. In a non-limiting example, “about” 2% refers to 1.95% to 2.05%.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Administration: To provide or give a subject an agent by any effective route. Exemplary routes of administration include, but are not limited to, topical, oral, injection (such as subcutaneous, intramuscular, intradermal, intraperitoneal, intravenous, and intratumoral), sublingual, transdermal, intranasal, topical and inhalation routes.

Anti-oxidant composition: A composition that has anti-oxidant activity.

Anti-oxidative effective amount: An amount sufficient to induce an anti-oxidant effect in a subject to whom the amount of a composition is administered.

Cleanser: A skin care product that is formulated to remove cosmetics, dead skin cells, oil, dirt and other loose material from the surface of the skin. A cleanser is generally the first component in a skin regimen, and is used prior to application of a toner, serum, and/or moisturizer. A cleanser can be a gel, which can liquefy upon contact with the skin, or an emulsion of oil and water. A cleanser can have various forms, including but not limited to, gels, liquids, wipes, creams or lotions, and can be foaming or non-foaming.

Combination: Two or more separate components, generally included in separate containers, that act together to improve the appearance of the skin, when they are applied to the skin in sequence. A combination can include two, three, four, five or six separate compositions (components). A combination can include a toner composition, a day moisturizer composition, a night moisturizer composition, a cleanser composition and a serum composition.

Day Moisturizer: A moisturizer that contains a sunscreen, such as a sunscreen with a sun protection factor (SPF) rating such as about 20, about 30, about 40 or about 50. A day moisturizer is generally the last component in a morning skin regimen, and can be used after application of a cleanser, toner and/or serum, and prior to the application of makeup.

Decolorized: A “decolorized” extract refers to one having less color than a reference, and it does not require an absence of coloration. A “decolorized” extract as used in this specification refers to the extract having a lowered level of condensed tannins and a less dark coloration of the type associated with condensed tannins in red grapes, red grape extracts, and red wine.

Effective Amount: An amount of a composition that alone, or together with an additional agent(s) (for example, additional anti-oxidant compounds), induces the desired response. The effective amount can be administered in a single dose, or in several doses, for example daily. However, the effective amount can depend on the subject being treated, the type of the condition being treated, and the manner of administration.

Excipient: An inactive substance used as a carrier for the active ingredients of a composition. Excipients can include substances that are used to bulk up formulations with active ingredients, allow for convenient and accurate dosage, stabilize the active ingredients, and make the delivery system optically and/or organoleptically acceptable. Examples of pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like.

Extract: A solution or other preparation of at least some of the active ingredients of a plant or one or more of its parts, such as its fruit or seeds. The extracts disclosed herein are solvent extracts, in which seeds are exposed to a liquid extract solvent (such as heated water) to remove active principles from the seeds. An extract initially obtained by solvent extraction may be converted into a dried form and still be considered an “extract.” An “aqueous” or “water” extract refers to an extract obtained by solvent extraction with water and no other solvent (such as ethanol).

Flavonoids: A class of plant secondary metabolites. Flavonoids constitute an important group of dietary polyphenolic compounds that are widely distributed in plants. More than 4000 chemically unique flavonoids have been identified in plant sources, such as fruits, vegetables, legumes, nuts, seeds, herbs, spices, flowers, as well as in beverages such as tea, cocoa, beer, wine, and grape juice.

On a dry weight basis, grape seeds contain about 4 to about 8% flavonoids. Flavonoids in grape seeds refer primarily to flavan-3-ols, specifically (+)-catechin, (−)-epicatechin, and (−)-epicatechin 3-gallate, and complexes thereof. The flavan-3-ols in grape seeds are present in monomeric, oligomeric or polymeric forms. Two or more chemically linked flavan-3-ol monomers are called proanthocyanidins or oligomeric proanthocyanidins (“OPCs”), which includes procyanidins and prodelphinidins. OPCs containing two monomers are called dimers, three monomers are called trimers, four monomers are called tetramers, five monomers are called pentamers, etc. The oligomers have chain lengths of 2 to 10; polymers represent components with chain lengths greater than 10. Thus, oligomers in grape seed extracts include, for instance, dimers and trimers, and there is evidence that the polymers can have as many as 50 to 100 units.

Flavonoids are present in all parts of the grape, including the skin, juice and pulp, and not just in the grape seed. In order for polyphenolic compounds to be used commercially as a grape extract, including as grape seed or grape pomace extracts, these compounds have to be separated from grapes in a more concentrated form. Extensive research suggests that grape extracts, such as grape seed extracts, are beneficial in many areas of health because of its promoting youthful skin, cell health, elasticity, and flexibility. Other studies have shown that proanthocyanidins help to protect the body from sun damage, to improve vision, to improve flexibility in joints, arteries, and body tissues such as the heart, and to improve blood circulation by strengthening capillaries, arteries, and veins.

Improving the appearance of skin: Effecting a visually and/or physically measurable benefit in skin appearance and/or feel, which can include reducing, preventing, or delaying the appearance of dermatological signs of age in the skin. Dermatological signs of skin aging include skin wrinkling, thinning of the dermal layers, reduced elasticity and hydration levels, and increased pigmentation and redness. Benefits that may be provided using the disclosed compositions and methods include, but are not limited to, a reduction in the depth, length, width, or number of skin wrinkles; a reduction in skin roughness, skin sagging, pore size, or skin hyperpigmentation; and an increase in skin smoothness, radiance or luminosity, firmness, elasticity, or the evenness of skin tone or coloration.

Moisturizer: A skin care product that is formulated to add moisture to the external layers of the skin, and can act as a vehicle for topical delivery of ingredients to the skin. A moisturizer can have different modes of action, including by forming a film on the surface of the skin to reduce moisture loss, by attracting water vapor from the air to add moisture, or by adding moisturizing factors to the skin surface. A moisturizer can be an emulsion, or a viscous semi-solid.

Muscadine Grape (Vitis rotundifolia): Grapes native to the southeastern United States, and found in the wild from Delaware to the Gulf of Mexico and westward to Missouri, Kansas, Oklahoma, and Texas. Muscadines are well adapted to the warm, humid conditions of the southeastern United States. The fruit is borne in small, loose clusters of 3 to 40 grapes, quite unlike the large, tight bunches characteristic of European and American grapes. The round, 1 to 1½ inch fruits have a thick, tough skin and contain up to 5 hard, oblong seeds. In color the fruits range from greenish bronze through bronze, pinkish red, purple and almost black. They are considered a red grape as the term is used in this specification.

Many different varieties of muscadine grapes are available, including female (pistillate) varieties such as Black Beauty, Black Fry, Darlene, Fry, Higgins, Jumbo, Scuppernong, Sugargate, Summit, Supreme, and Sweet Jenny, and self-fertile varieties such as Carlos, Cowart, Dixieland, Dixie Red, Fry Seedless, Magnolia, Nesbitt, Noble, Redgate, Regale and Sterling.

For example the bronze colored varieties of muscadine grapes are identified by those skilled in the art as including Carlos, Chowan, Doreen, Higgins, Magnolia, Nevermiss, Pamlico, Roanoke, Scuppernong, Sterling, and Summit cultivars. Purple varieties are darker skinned then the bronze colored varieties and include Albermarle, Bountiful, Cowart, GA-1, Hunt, NC-1, Noble, Regale, Tarheel, and Jumbo. Some of the purple varieties are also referred to as black colored.

The phytochemical constituents of the whole muscadine grape differ from Vitis vinifera. Muscadines have a higher total phenolic content distinguished by high ellagic, gallic, and flavonoid glycoside concentrations. The presence of ellagic acid in muscadine grapes is unique and is found in the form of free ellagic acid, ellagic acid glycosides, methoxylated derivatives and ellagitannins Another unique feature is the anthocyanin chemistries observed in muscadines. Present as 3,5-diglucosides (as opposed to 3-glucosides) of delphinidin, cyanidin, petunidin, peonidin, and malvidin in non-acylated forms, these compounds and the natural color influence from other anthocyanins present within the grape impart a dark purple color to juice and pomace obtained from the purple varieties. Purple pomace extracts contain anthocyanins while bronze pomace extracts do not.

The red and purple colored anthocyanins found in bronze and purple muscadine grapes are polyphenolic compounds that have anti-oxidant properties. Purple and bronze muscadine grapes contain several other flavonoid classes of polyphenols with flavan-3-ols and their oligomers being the most abundant class and flavonols being the second most abundant flavonoids present in whole muscadines. The major phenolics reported for the muscadine skin fraction (in descending order) are ellagic acid, myricetin, quercetin, and kaempferol while those reported for seeds are epicatechin, catechin and gallic acid (Pastrana-Bonilla et al. J. Agric. Food Chem. 51:5497-5503, 2003).

A muscadine grape contains pomace and juice. “Other than the whole grape” includes a muscadine grape from which at least some of the juice has been extracted, and in some examples includes less than about 95% or less than about 90% of the original juice in the grape.

Night Moisturizer: A moisturizer that does not contain a chemical sunscreen. A night moisturizer is generally the last component in an evening skin regimen, and can be used after application of a cleanser, toner and/or serum.

Pharmaceutically Acceptable Vehicles: The pharmaceutically acceptable vehicles (carriers) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more compositions, such as one or more grape extract compositions, and additional naturally or non-naturally occurring pharmaceutical agents that would not be found with the grape extracts in nature. The use of pharmaceutically acceptable carriers does not imply that that product so made is useful only for pharmaceutical purposes. Rather it implies that the product is suitable for administration to or consumption by a subject, for example, in a topical composition. In particular embodiments, the vehicle is a carrier for a topical composition, such as a liquid, gel, foam, ointment or lotion. Particularly useful vehicles are those that are pharmaceutically or cosmetically acceptable for topical applications, such as one or more aqueous systems, glycerin, C₁₋₄ alcohols, fatty alcohols, fatty ethers, fatty esters, polyols, glycols, vegetable oils, mineral oils, liposomes, laminar lipid materials, silicone oils, water, or any combinations thereof.

For topical compositions, such as those disclosed herein, the muscadine grape extract compositions can be formulated in any suitable product form. Such product forms include, but are not limited to, aerosol spray, dispersion, emulsion, foam, gel, liquid, lotion, mousse, ointment, patch, pomade, powder, pump spray, solid, solution, stick, or towelette. The carrier can also be a variety of existing skin lotions, gels, creams, ointments, toners, cleansers, moisturizers or sun screens to which the muscadine grape extract is added in a desired concentration, for example about 0.025% to about 0.25% by weight.

Polyphenols: Polyphenols from grapes and cocoa have been found to enhance both cardiovascular function and cognitive health. Flavanols (also called flavan-3-ols) represent the majority of grape seed and cocoa polyphenols; this class of phenolic compounds ranges from monomeric species such as catechin and epicatechin to oligomers (often termed proanthocyanidins) to polymers (often termed tannins or condensed tannins) The term “phenolic” is used interchangeably with the term polyphenol in the art and in this specification.

Polyphenols are also present in other parts of the grape, including in the skin, juice and pulp. The highest concentration of polyphenols in a grape resides in the grape seeds. Grape seeds are waste products of the winery and grape juice industry. These seeds contain lipid, protein, carbohydrates, and about 4 to about 8% polyphenols (dry weight) depending on the variety. Grape seed extract can protect the body from premature aging, disease, and degeneration.

Pomace: The skins, seeds, and pulp remaining following juice extraction. In one example a pomace extract is a bronze muscadine pomace extract, a purple muscadine pomace extract or a combination thereof. Many different varieties of muscadine grape pomaces are available as starting materials, and they include female (pistillate) varieties such as Black Beauty, Black Fry, Darlene, Fry, Higgins, Jumbo, Scuppernong, Sugargate, Summit, Supreme, and Sweet Jenny, and self-fertile varieties such as Carlos, Cowart, Dixieland, Dixie Red, Fry Seedless, Magnolia, Nesbitt, Noble, Redgate, Regale and Sterling. Muscadine pomace contains phenolic compounds, including gallic acid and ellagic acid, having anti-oxidant properties.

The pomace can be present in a whole grape wherein the whole grape contains at least about 90% or at least about 95% of the juice of the grape, or the pomace can be substantially isolated and consist essentially only of the pomace once the grape has been compressed to remove the juice.

Pomace-only: The pomace portion of a grape from which juice has been removed, for example by compression of the grape. As used herein, “pomace-only” refers to a pomace that contains no more than about 1% juice as a percentage of its weight. In some embodiments, the pomace-only contains no more than about 0.5% juice as a percentage of its weight.

Purified: The term purified does not require absolute purity; rather, it is intended as a relative term. Thus, for example, a purified substance is one in which the substance is more enriched than the substance in its natural environment, for example in a fruit (e.g., grape). In one embodiment, a preparation is purified such that the substance represents at least about 5% (such as, but not limited to, at least about 10%, at least about 20%, at least about 30%, at least about 40%, at least about 50%, at least about 70%, at least about 80%, at least about 90%, at least about 95%, at least about 98% or at least about 99%) of the total content of the preparation.

Range: With respect to ranges, the term “in the range of x to y” or “from x to y” includes any value between x and y, as well as the endpoints x and y.

Red grapes: Red grapes are those from which red wine is generally made, and which have generally higher levels of tannins than white grapes from which white wine is made. “Red wine” is a general term for dark wines. A determination of the color of a wine can be made according to the International Organization of Vine and Wine which provides method to assess the color of a wine using a spectrophotometer and the calculation of indices in the Lab color space. Examples of grapes from which red wine is made include Syrah, Merlot, Cabernet Sauvignon, Malbec, Pinot Noir, Zinfandel, Sangiovese, Barbera, and Muscadine. Muscadine grapes (Vitis rotundifolia) range in color from bronze to dark purple to black in color when ripe.

Regimen: As used herein, a regimen is a systematic treatment practice to improve the appearance of skin. A regimen may include regular application to the skin of one or multiple components, such as a toner, day moisturizer, night moisturizer, cleanser and serum. The skin can be treated with one or all of the components once a day, or the components may be applied multiple times a day, such as in the morning and in the evening. For example, a day moisturizer may be applied in the morning and a night moisturizer may be applied in the evening. Generally, a regimen includes multiple applications.

Selective extraction: Selective extraction refers to preferential extraction of a target (such as condensed tannins) In some embodiments, selective extraction means that the target is the predominant species extracted.

Serum: A skin care product that is formulated to deliver ingredients deeply into the skin. As such, a serum generally contains higher concentrations of active ingredients, and is applied directly to clean skin prior to application of a moisturizer. A serum can have a low viscosity and a higher proportion of water than oil.

Skin care composition: A topical composition having active ingredients that can improve the health, aesthetic and/or cosmetic appearance of skin. Such improvements can be manifested, for example, by a reduction in dermatological signs of aging caused by factors such as chronological aging, hormonal aging, and photoaging; reduction in skin fragility, pore size reduction, loss of collagen and/or elastin; diminishing appearance and/or depth of lines and/or wrinkles including fine lines and/or wrinkles; reducing hyperpigmentation; improvement in skin tone, radiance, clarity and/or tautness; reducing skin sagging; promoting anti-oxidant activity; improving skin firmness, plumpness, texture, suppleness and/or softness; improvement in procollagen and/or collagen production.

Subject: Living multi-cellular vertebrate organisms, a category that includes both human and veterinary subjects, such as a companion animal, including a cat, dog or horse. A subject who follows a skin care regimen, or a method of caring for the skin, to improve the appearance of skin is a subject who may benefit from such an improvement, such as a subject who desires to offset tissue damage caused by reactive oxygen species, or decrease signs of aging or other conditions that are associated with aging skin.

Tannins: Naturally occurring polyphenolic biomolecules that bind to and precipitate proteins, amino acids and alkaloids. The term “tannin” originally referred to the use of wood tanning agents from oak that were used in tanning animal hides into leather. However, the term tannin is widely applied to a large polyphenolic compounds with molecular weights ranging from about 500 to over 3,000 (gallic acid esters) and up to about 20,000 (proanthocyanidins) that are found in plants, such as red grape seeds, seeds and stems. In terms of their chemical composition, plant tannins are divided into the hydrolysable tannins and the condensed tannins or flavonoids. The hydrolysable tannins are polymerized simple phenolic substances, such as esters of gallic acid and its dimers (digallic acid, ellagic acid). The hydrolysable tannin can be further divided into gallotannins that yield gallic acid after hydrolysis, and ellagitannins that release ellagic acid after hydrolysis. In contrast to the hydrolysable tannins, the condensed tannins are not decomposable by hydrolysis. On the contrary, when subjected to heating in an acidic medium, they progressively polymerize and form amorphous anthocyanin pigments of red color, or insoluble yellow-brown products, of high molecular mass, called phlobaphenes.

Toner; A skin care product that is formulated to cleanse the skin and reduce pore size. In some embodiments, a toner can be applied after a cleanser and prior to application of a serum or moisturizer. A toner can have a low viscosity and a higher proportion of water than oil.

Topical application: A topically applied agent is applied only in a specific surface area of the skin, and not throughout the body. In particular examples, the composition is applied to the skin in an area where an improved effect is desired, such as the crow's feet areas around the eye. For example, the composition can be topically applied to facial skin. A topical composition that is intended for application to the skin is a “skin composition.”

Ultrafiltration: A type of membrane filtration in which forces (such as pressure or concentration gradients) lead to a separation through a semipermeable membrane. Ultrafiltration membranes are typically characterized by the molecular weight cut off (MWCO) of the membrane. Suspended solids and solutes of higher molecular weight are retained in the retentate, while water and lower molecular weight solutes pass through the membrane in the permeate. Different types of modules can be used for ultrafiltration processes. Examples of such modules are tubular elements that use polymeric membranes cast on the inside of plastic or paper tubes; hollow fiber designs that contain multiple hollow fibers; spiral wound modules in which flat membrane sheets are separated by a thin meshed spacer material that is rolled around a central perforated tube and fitted into a tubular steel pressure vessel casing; and plate and frame assemblies that use a membrane placed on a flat plate separated by a mesh like material through which the filtrate passes.

Unit dose: A physically discrete unit containing a predetermined quantity of an active material calculated to individually or collectively produce a desired effect, such as a therapeutic effect. A single unit dose or a plurality of unit doses can be used to provide the desired effect or activity, such as an improvement in the appearance of skin. In one example, a unit dose includes a desired amount of an anti-oxidant. In a different example, a unit dose includes a desired amount of an agent that reduces the depth of wrinkles, the number of wrinkles, the length of wrinkles, the width of wrinkles, the amount of skin roughness, skin sagging, pore size, or skin hyperpigmentation; increases skin smoothness, skin radiance or luminosity, skin firmness, the evenness of skin tone; or any combination thereof. In another example, the unit dosage form contains multiple predetermined dosages of the active material.

### II. COMBINATIONS USED IN A SKIN CARE REGIMEN

Given the known effects of age and exposure to damaging stimuli on the skin, including from ROS, sunlight, and pollution, there is a need for effective products that provide protection from these harmful factors and improve the appearance of the skin. The five components used in the skin care regimens and methods disclosed herein, achieve these objectives by combining several ingredients in a consumer-acceptable form, which at the same time effectively improves the appearance of skin upon application.

Each of the five components of the combinations described herein include a decolorized muscadine pomace solvent extract having a reduced content of condensed tannins, beta-glucan and grape seed extract. Topical compositions made with decolorized muscadine pomace solvent extracts have a polyphenol profile that is beneficial to the skin, but the compositions are relatively decolorized because of their lower condensed tannin content. As used herein, a “low condensed tannin content” means a condensed tannin content of about 10% or less by weight. The compositions include a decolorized muscadine pomace extract, beta-glucan, and grape seed extract.

The compositions are used in a five component system (combination) that improves the appearance of the skin, such as a reducing in the depth, length, width, or number of wrinkles, reducing skin roughness, reducing skin sagging, increasing elasticity, reducing pore size, reducing skin hyperpigmentation, increasing skin smoothness, increasing skin radiance, increasing skin luminosity, increasing skin firmness, evening skin tone and/or evening skin coloration. These compositions are applied sequentially.

A. Decolorized Muscadine Pomace Solvent Extracts

The compositions used in the combinations and methods disclosed herein, contain a decolorized muscadine pomace extract that has a lower level of condensed tannin content as compared to a muscadine pomace extract that has not been decolorized. Tannins are widely distributed in many types of plants. Tannins can be found in leaf, bud, seed, root and stem tissues. Tannins are found in monocots (44 families, according to Wikipedia) and dicots (180 families, according to Wikipedia), and are found in gymnosperms and angiosperms. All species of the following dicots contain tannins: Aceraceae, Actinidiaceae, Anacardiaceae, Bixaceae, Burseraceae, Combretaceae, Dipterocarpaceae, Ericaceae, Grossulariaceae, and Myricaceae. All species of Najadaceae and Typhaceae (monocots), also contain tannins. Condensed tannins (including proanthocyanidins, polyflavonoid tannins, catechol-type tannins, pyrocatechollic type tannins, non-hydrolysable tannins or flavolans) are found in plant species such as Ltihcarpus glaber, Prunus sp, Schinopsis lorentzil, Acacia mollissima, Vitis vinifera, and Commiphora angioensis. Pine barks and spruce barks contain condensed tannins Vascular plants (e.g., Tracheophytes, vascular plants) also include condensed tannins. The decolorized plant extracts have a lower condensed tannin content as compared to non-decolorized precursor extracts of any of these plants that contain condensed tannins.

In some embodiments, the plant extracts are grape pomace extracts, such as, but not limited to, muscadine (Vitis rotundifolia) grape pomace extracts that contain condensed tannins. In some non-limiting examples, the plant extracts are red grape pomace extracts that contain condensed tannins. In certain examples, the decolorized muscadine pomace solvent extract is made from a liquid muscadine pomace extract that is a combined extract of bronze and purple muscadine grapes, having an enhanced solubility of ellagic acid (which is unique to muscadine grapes). The combined muscadine grape pomace precursor extract obtained by any of the processes disclosed herein, has levels of condensed tannins that impart dark coloration to the extract.

As discussed in applicant's disclosures of PCT Publication No. WO 2010/014870, PCT Publication No. WO 2010/014873, U.S. Pat. No. 8,568,804, U.S. Pat. No. 9,132,162 and U.S. Pat. No. 9,173,916 (all five of which are also incorporated herein by reference), muscadine extracts with improved ellagic acid solubility can be obtained by combining bronze and purple muscadine pomace extracts. Various methods of making the combined extracts were disclosed, such as combining a bronze muscadine pomace extract with a purple muscadine pomace extract to produce a muscadine pomace extract, wherein the ratio of bronze muscadine pomace extract to purple muscadine pomace extract ranges from about 0.1 to about 10 (weight to weight), such as about 0.3 to about 3 (weight to weight). In certain examples, the decolorized muscadine pomace solvent extract is prepared from such a combined precursor extract.

The combined precursor extract can be made by separate extraction of bronze and purple muscadine grapes with subsequent combination of the extracts, or by simultaneous extraction of bronze and purple muscadine grapes combined in desired ratios. In the disclosed examples, the decolorized muscadine pomace solvent extract is an aqueous extract of the precursor combined liquid muscadine pomace.

PCT Publication No. WO 2010/014870, PCT Publication No. WO 2010/01487, U.S. Pat. No. 8,568,804. U.S. Pat. No. 9,132,162 and U.S. Pat. No. 9,173,916 disclose that the precursor muscadine pomace extract compositions have improved anti-oxidant activity. Methods of producing compositions containing decolorized muscadine pomace solvent extracts include combining a muscadine (Vitis rotundifolia) pomace extract having a polyphenol content of at least about 2% and trans-resveratrol from a source other than muscadine with a minimum purity of at least about 5%, wherein a ratio of muscadine polyphenols to trans-resveratrol is in the range of about 0.1/1 to about 10/1 (weight to weight). Methods of producing the compositions containing decolorized muscadine pomace solvent extracts include combining a muscadine (Vitis rotundifolia) pomace extract having a polyphenol content of at least about 2% and trans-resveratrol from a source other than muscadine with a minimum purity of at least about 5%, wherein a ratio of muscadine pomace extract to trans-resveratrol is in the range of about 0.2/1 to about 50/1 (weight to weight), such as about 5/1 to about 50/1 (weight to weight) including about 20/1 to about 50/1 (weight to weight), such as about 18 to about 1 (weight to weight), thereby producing a muscadine pomace extract and trans-resveratrol mixture with anti-oxidant activity.

In some embodiments, the ratio of bronze to purple muscadine pomace extract in the precursor liquid extract ranges from about 0.1 to about 10, such as about 0.3 to about 3. For example, the ratio of bronze muscadine pomace extract to purple muscadine pomace extract is about 2.75 to about 1 (weight to weight), about 2.5 to about 1 (weight to weight), about 2.25 to about 1 (weight to weight), about 2 to about 1 (weight to weight), about 1.5 to about 1 (weight to weight), or about 1 to about 1 (weight to weight). In other examples, the ratio is about 10 to about 1, about 7.5 to about 1, or about 5 to about 1. As used herein the term “about” is defined as ±0.5. In a particular example, the ratio of bronze muscadine pomace extract to purple muscadine pomace extract is about 2.25 to about 1 (weight to weight).

In certain embodiments, the combined precursor muscadine (Vitis rotundifolia) pomace liquid extract has a polyphenol content of at least about 2%. For example, the polyphenol content is at least about 3%, at least about 3.5%, at least about 4%, at least about 4.5%, at least about 5%, at least about 6%, at least about 7%, at least about 8%, at least about 10%, at least about 12%, or at least about 14%. In a particular example, the combined precursor muscadine (Vitis rotundifolia) pomace liquid extract has a polyphenol content of about 4%.

In some embodiments, the disclosed combined precursor liquid muscadine pomace extracts include about 20% to about 50% solids, such as at least about 25%, at least about 30%, at least about 35%, at least about 37%, at least about 40%, at least about 42%, at least about 44%, at least about 46% or at least about 48%, in a liquid. In a particular example, the combined precursor extract includes about 40% solids in a liquid.

The liquid pomace precursor extracts can be obtained by any extraction method, such as pressing under pressure or extracting with a solvent. Particular examples are solvent extraction, for example with alcohol, water (such as heated water), or a combination of alcohol and water.

The extract can further be fermented to remove extracted sugars. In one example, fermentation is performed following extracting the bronze muscadine pomace and purple muscadine pomace but prior to combining the bronze muscadine pomace extract with the purple muscadine pomace extract to produce a liquid muscadine pomace extract. In other examples, fermentation is performed after combining the bronze muscadine pomace extract with purple muscadine pomace extract in the desired post extraction ratio (such as, but not limited to, at about a 2:25 to about 1 bronze to purple ratio).

Fermentation may be performed by any method known to one of skill in art. For example, yeast and yeast nutrients can be added to the pomace and fermentation continued until the residual sugar content is converted to ethanol. In one example, two pounds of yeast are added per 1000 gallons of 1× (unconcentrated) extract; fermentation is typically complete after three days. In other examples, the amount and/or strain of yeast and duration and temperature of fermentation may vary according to individual methods known to one of skill in art. In some examples, enzymes are used to clarify and/or settle residues or to improve extraction yield in the pomace extracts. Examples of such enzymes include pectinase or a blend of enzymes from Aspergillus niger that are commercially available from sources such as Scott Laboratories. These enzymes may be added to the pomace extract before or during fermentation.

In some embodiments, the bronze muscadine pomace extracts and purple muscadine pomace extracts are filtered prior to and/or following fermentation. Filtration can be performed according to general methods known to those of skill in the art. In a particular example, extracts are filtered through sieves of appropriate mesh size, such as USP mesh (typically 120 mesh) or a similar cloth filter (for example filters commercially available from Millipore Corporation).

In certain embodiments, preparation of the liquid muscadine pomace extracts further includes concentrating the bronze muscadine pomace extract and the purple muscadine pomace extract so that each extract includes about 20% to about 50% solids, such as at least about 25%, at least about 30%, at least about 35%, at least about 37%, at least about 40%, at least about 42%, at least about 44%, at least about 46% or at least about 48%, in a liquid. In a particular example, the extracts are concentrated so that each extract includes about 40% solids in a liquid. Generally known methods for concentrating samples, including methods for concentrating samples disclosed herein, can be used to concentrate the bronze and purple extracts.

In any of the foregoing examples of preparing the decolorized muscadine pomace solvent extract, whole purple grapes can be substituted for purple pomace. The purple muscadine pomace extract can include (a) an extract of whole purple muscadine grapes; (b) an extract of purple muscadine pomace from other than whole grapes; or (c) a mixture of (a) and (b). The whole purple grapes contain grape juice that is a source of anthocyanins from other than the grape pomace, and when solvent extracted from the whole grape, these anthocyanins have been found to surprisingly enhance solubility of ellagic acid in the mixture of bronze and purple muscadine grape extracts. However, additional sources of anthocyanins can also be provided by a colored fruit or a byproduct of a colored fruit other than purple muscadine grapes, such as a blueberry, blackberry or raspberry. In some examples, the anthocyanin is from a product of a fruit processing stream such as a juice concentrate or a by-product of a fruit processing stream such as fruit skins that are separated from a fruit puree processing stream.

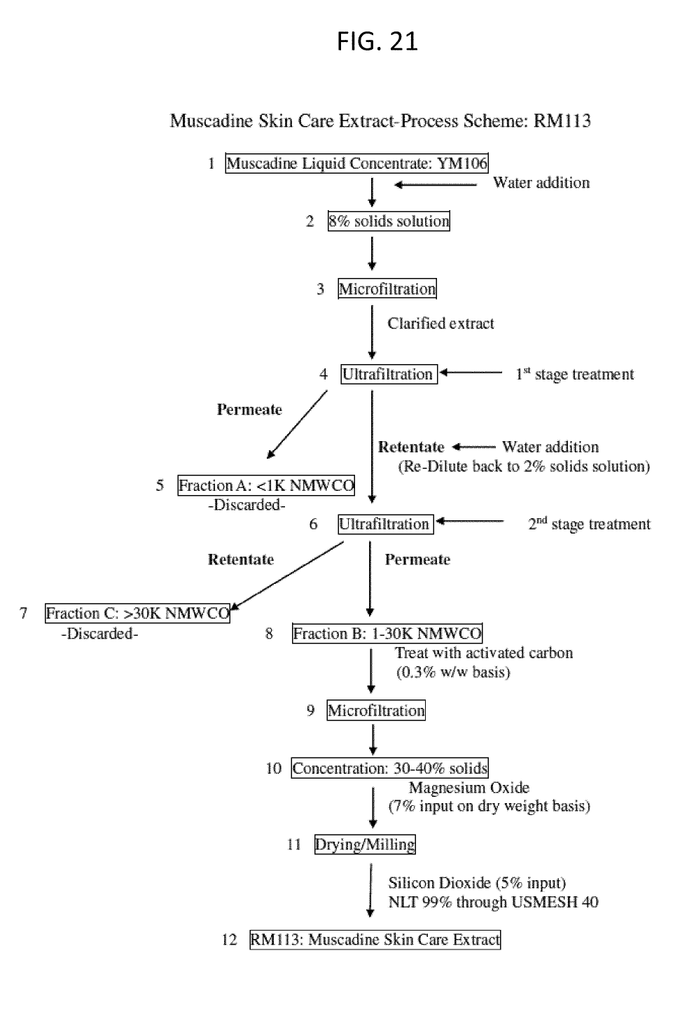

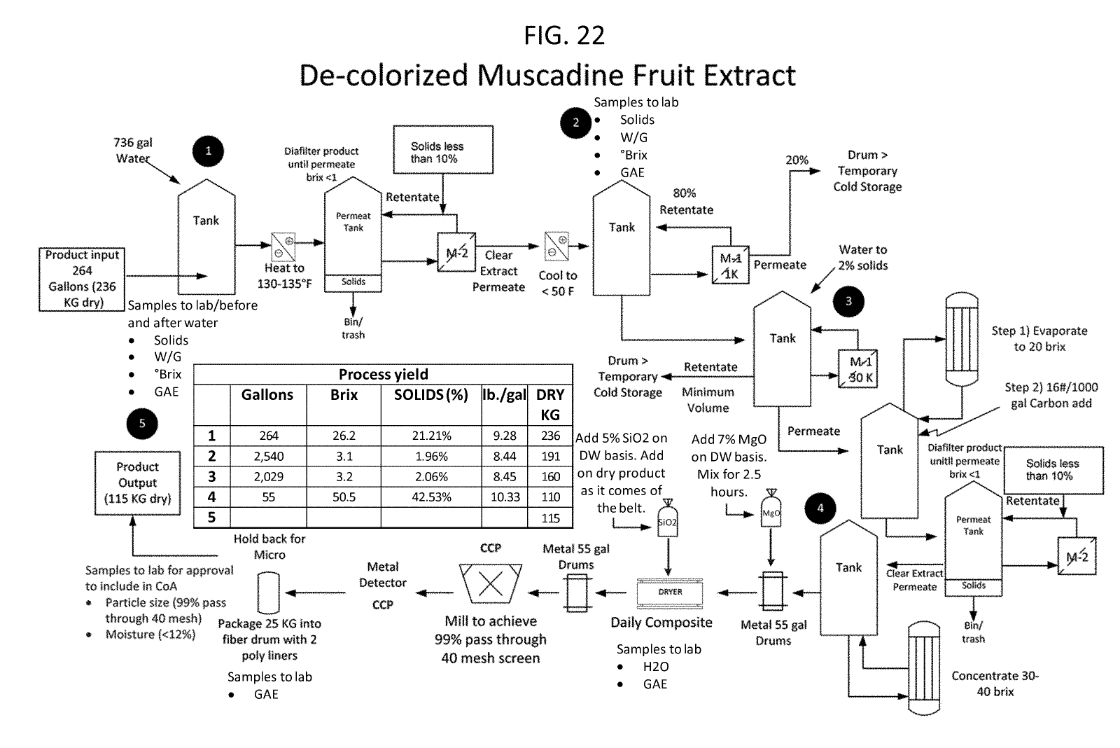

A decolorized muscadine pomace solvent extract can be prepared from a precursor extract by subjecting the precursor extract to a decolorization process. An example of a decolorization process for reducing the tannin content of the muscadine grape pomace extract is shown in FIG. 21. A precursor material is a muscadine pomace extract liquid concentrate 1. In this example, the concentrate 1 is about 40 to about 45% solids, as described in U.S. Pat. Nos. 8,512,771 and 9,173,916. Concentrate 1 is diluted to less than about 10% solids by adding purified water to produce an about 8% solids solution 2 (the percent solids can range from about 2 to about 10%). Solution 2 is heated to about 130 to about 135° F., and passed through a microfiltration device 3 to clarify the material. The microfiltration process is more efficient when the precursor material is diluted to a very low level of solids (about 2 to about 5%) and heating also helps facilitate the process. In this specific example, the microfiltration device 3 is a mechanical filter of 0.45 micron pore size and NMWCO of 200,000 daltons (or 200 kDa) which removes microorganisms and suspended particles from solution in preparation for a subsequent ultrafiltration treatment 4. The clarified extract is quickly cooled down to <50° F. in a holding tank before starting the 1′ stage ultrafiltration step.

The cooled and clarified extract is then subjected to ultrafiltration treatment 4 with standard nominal molecular weight cutoff ultrafiltration membrane of 1000 daltons (or 1 kDa) yielding a permeate (Fraction A) at 5. Fraction A includes compounds having molecular weights of less than about 1000 and contains mainly organic acids, sugars, and minerals but surprisingly contains less than about 1% phenolic compounds. These phenolic compounds include trace amounts of phenolic acids and flavonoids such as gallic acid and quercetin. Although molecules having a molecular weight of up to about 1,000 daltons are able to permeate the ultrafiltration membrane, it was unexpectedly found that this fraction contained a very low phenolic acid content, perhaps due to physicochemical interactions between the phenolic acids and other molecules, possibly fiber, contained in the precursor material. This unexpected finding can be obtained with the use of standard nominal molecular weight cutoff ultrafiltration membranes as low as about 500 daltons and up to about 5000 (or 5 kDa). Fraction A could serve as a “muscadine” type flavor component in other products, but this material is discarded in the process of preparing the decolorized muscadine pomace solvent extract used for the topical compositions described herein.

The retentate (material that does not pass through the ultrafiltration membrane) remaining from the low molecular weight ultrafiltration (described above) is reconstituted in purified water to approximately 2% solids concentration and subjected to a second ultrafiltration process 6 with a 30 kDa cutoff ultrafiltration membrane. The resulting retentate 7 (Fraction C) is discarded and the permeate 8 (Fraction B) containing the decolorized muscadine pomace extract is retained for further processing into the disclosed topical compositions. Fraction C primarily contains polymerized condensed tannins, protein, and fiber. In contrast, Fraction B is enriched with polyphenols (about 9 to about 10% dry weight), low in sugars (less than about 4% dry weight) and greatly reduced in polymeric condensed tannin content (undergoing a minimum reduction of about 70% of the initial condensed tannin content). Although molecules having a molecular weight of up to about 30,000 daltons are able to permeate the ultrafiltration membrane, it was unexpectedly found that at least about 70% of the polymeric condensed tannins remained in Fraction C, possibly owing to physicochemical interactions. This unexpected finding may result from the use of standard nominal molecular weight cutoff ultrafiltration membranes as low as about 25,000 daltons (25 kDa) and up to about 100,000 daltons (or 100 kDa).

Fraction B also contains fiber (about 1.0%), protein (about 7.8%), fat (about 0.1%) and organic acids (about 16.0%). Fraction C, which is enriched in polymeric condensed tannins, could serve as a source of anti-oxidants for use in a variety of products, but this material is discarded in the preparation process of the decolorized muscadine pomace solvent extract. At this point in the decolorization process, the muscadine fruit extract, Fraction B, has minimal sugar content (less than about 4% dry weight) and is greatly reduced in polymeric condensed tannins Fraction B is useful for skin care products because it contains powerful polyphenols yet has little or no potential for brown discoloration owing to the removal of condensed tannins and its minimal sugar content.

When introduced into various formulations for topical use, brown color undertones are not detected.

Permeate 8 (Fraction B) is further processed by treating it with activated carbon at an about 0.3% weight to weight basis to remove odiferous components that are undesirable in a skin care product (buttery/yeasty notes). After treatment with activated carbon, Fraction B is subjected to a final microfiltration process 9 to remove all traces of activated carbon. After microfiltration the solution is concentrated 10 to about 30 to about 40% solids before mineralization with magnesium oxide to facilitate a free flowing powder after drying. Magnesium oxide is added in at an input of about 7.0% on a dry weight basis to the concentrate to completely facilitate the mineralization process, so as to form the magnesium salts of all the organic acids that exist in the extract. Finally, the product is subjected to drying/milling 11 to provide a uniform particle size. Silicon dioxide is added during this milling stage to prevent product caking.

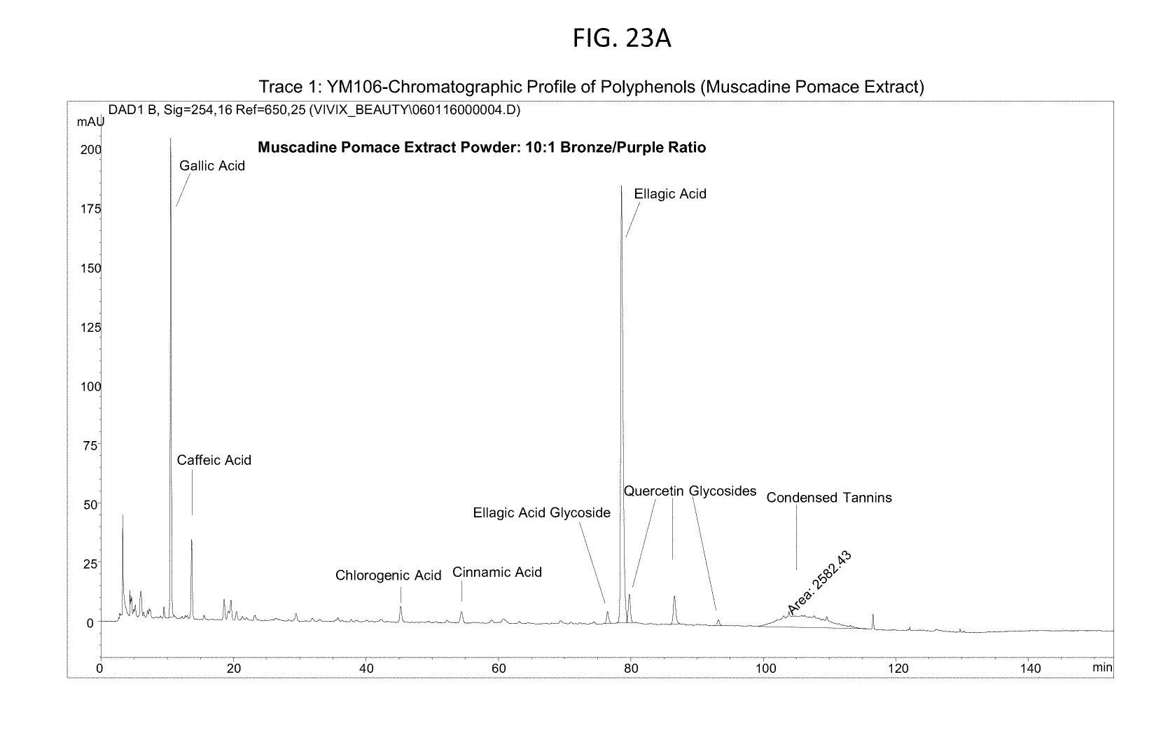

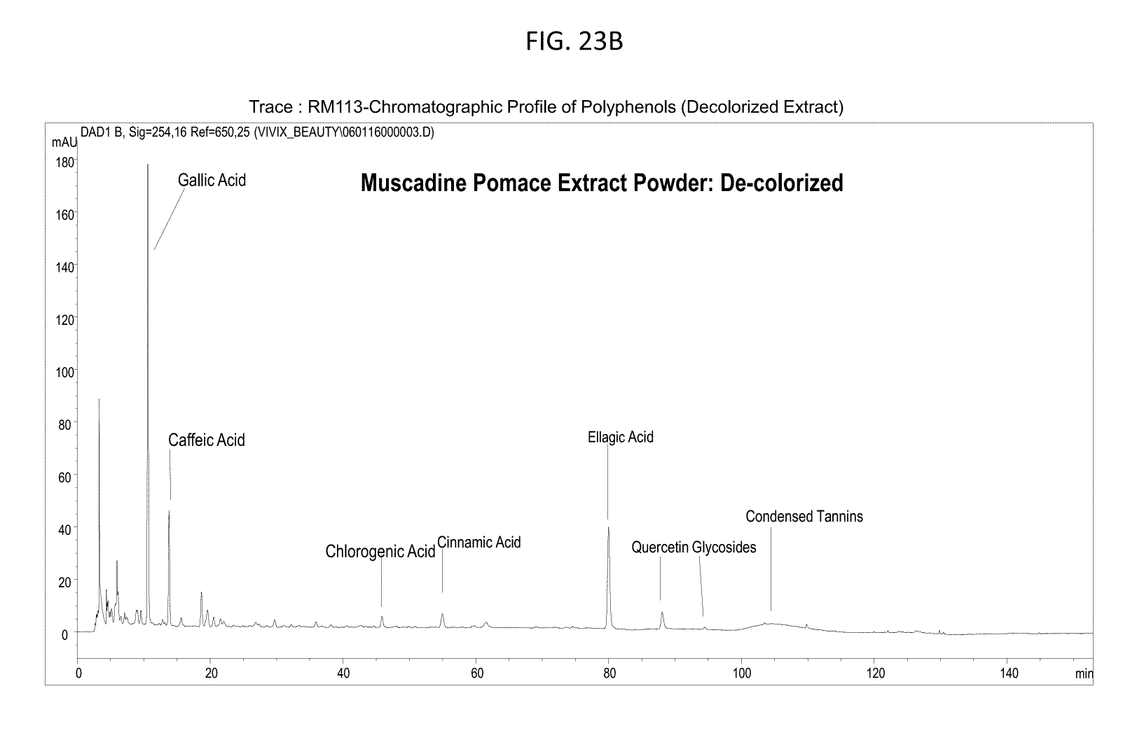

Compared to the precursor material, Fraction B has a mass reduction of about 75 to about 80% yet only about 35 to about 40% polyphenol content reduction when subjected to the above described process. Therefore, when comparing Fraction B to the precursor material, on an equivalent mass basis, the resulting reduction of non-tannic polyphenol content in Fraction B is only about 18 to about 20%. Thus, the predominant loss of polyphenols in Fraction B is attributed to the chemical class of polymeric condensed tannins; virtually no loss of other polyphenol chemical classes was detected (see chromatograms in FIG. 23). Thus, Fraction B contains, on average, about 9% muscadine polyphenols (predominantly non-tannic) whereas, the precursor material contains, on average, about 14% polyphenols consisting of both tannic and non-tannic muscadine polyphenols. Tables 1-3 depict the nutritional and polyphenol content comparisons and FIG. 23 provides a chromatographic comparison, for precursor material versus the decolorized extract produced by the above described process.

Table 1 compares the nutritional profiles (presented as % of dry extract) of the precursor and decolorized extracts. While the nutritional profiles are quite similar, owing to the mass reduction of the precursor material, there is some concentration of nutrient classes such as protein and simple sugars. Nevertheless, the final simple sugar (monosaccharide) content of the decolorized extract is quite low (less than about 4 to less than about 5%) and thus suitable for use in a topically applied product.

TABLE 1
Nutritional Profiles of Precursor and Decolorized Muscadine Extracts.

| | Precursor Extract | Decolorized Extract |
|---|---|---|
| Compound Class | (% dry weight) | (% dry weight) |
| Protein | 4.6 | 7.8 |
| Fat | 1.0 | 0.1 |
| Ash (Inorganic Constituents) | 16.0 | 18.9 |
| Sugar Acids | 16.5 | 16.5 |
| Soluble Fiber | 0.0 | 0.9 |
| Insoluble Fiber | 2.0 | 0.1 |
| Organic Acids | 23.0 | 16.0 |
| Sugar Alcohols | 3.9 | 8.8 |
| Polyphenols | 14 (Avg. G.A.E.) | 9 (Avg. G.A.E.) |
| Simple Sugars | 0.5 | 3.5 |
| Moisture | 12.0 | 10.0 |
| Amino Acids | 3.9 | 7.8 |
| “other” | 2.6 | 0.6 |
| Total | 100.0 | 100.0 |

Table 2 shows, on a dry weight basis, the total polyphenol content of each extract and further divides the polyphenol content into condensed tannin content and content of polyphenols other than condensed tannins. As shown in Table 2, while the total polyphenol content is about 35% lower in the decolorized extract (90 versus 140 mg/g dry weight), the condensed tannin content of the decolorized extract is at least about 70% lower than that found in the precursor extract (6 versus 21 mg/g dry weight). Thus, the majority (roughly 80%) of the polyphenol loss is attributable to the removal of condensed tannins confirming the preferential removal of condensed tannins versus other polyphenols using the process described above.

TABLE 2
Polyphenol content of Precursor and Decolorized Muscadine Extracts.
Phenolic Profiling

| | | | | De- | |
|---|---|---|---|---|---|
| | | Precursor | | colorized | |
| | Precursor | (% of total | Decolorized | (% of total | |
| Phenolic | (mg/g | poly- | (mg/g | poly- | Analysis |
| Class | extract) | phenols) | extract) | phenols) | Method |
| All other | 119.0 | 85.0 | 84.0 | 93.3 | (G.A.E. |
| Poly- | | | | | minus |
| phenols | | | | | condensed |
| | | | | | tannins)* |
| Con- | 21.0 | 15.0 | 6.0 | 6.7 | n-Butanol- |
| densed | | | | | HCL |
| Tannins | | | | | assay |
| Total | 140 | 100 | 90 | 100 | G.A.E |
| Poly- | | | | | Assay |
| phenols | | | | | |

*Gallic Acid Equivalents assay (Total Polyphenols via UV/VIS) − Condensed Tannins assay via UV/VIS = non-condensed tannins content.

The decolorized extract retains all the core polyphenols (listed in Table 4) found in the precursor material (phenolic acids, flavonoids, anthocyanins, and hydrolysable tannins) as shown in the chromatograms in FIG. 23 and further illustrated in Table 3 wherein the core/hallmark muscadine polyphenols are profiled. Using analytical methods, approximately 30% of the total polyphenols in the precursor extract (43.84 mg out of 140 mg) were measured as single phenolic classes and/or compounds and were compared to the corresponding classes/compounds in the decolorized extract.

As shown in Table 3, important (and in some cases unique) muscadine polyphenol classes such as phenolic acids, ellagic acid, gallic acid, ellagitannins, and gallotannins were detectable in the decolorized extract; in some cases, the percentage of selected compounds, e.g., gallic acid, increased. Thus, the primary difference in polyphenol content between the two extracts is the about 70% reduction in condensed tannin content. In summary, the reduction of condensed tannin content together with the low level of simple sugars renders decolorized Fraction B to be amenable for skin care applications when added in the typical usage amounts of topical formulations. These formulations are of use for skin care, and are more stable when stored, so that the product does not turn brown or darken in color over time.

TABLE 3
Polyphenol Profiles of Precursor and Decolorized Muscadine Extracts.

| | | Precursor | | Decolorized | |
|---|---|---|---|---|---|
| | Precursor | (% of | | (% of | |
| | (mg/g | identified | Decolorized | identified | |
| Phenolic Class | extract) | polyphenols) | (mg/g extract) | polyphenols) | Analysis Method |
| Ellagic Acid | 2.78 | 6.3 | 0.56 | 2.6 | HPLC/MS |
| Ellagic Acid | 0.68 | 1.6 | 0.67 | 3.1 | Quantitation w/ |
| Glycosides | | | | | Respective Standards |
| Gallic Acid | 7.58 | 17.3 | 6.55 | 30.7 | |
| Quercetin/ | 1.35 | 3.1 | 0.55 | 2.6 | |
| Glycosides | | | | | |
| Gallotannins | 2.21 | 5.1 | 2.16 | 10.1 | Hydrochloric Acid |
| Ellagitannins | 2.60 | 5.9 | 1.64 | 7.7 | Hydrolysis |
| | | | | | HPLC/MS |
| | | | | | Quantitation: Ellagic |
| | | | | | Acid and Methyl |
| | | | | | Gallate increase |
| Proanthocyanidins | 20.83 | 47.5 | 6.33 | 29.7 | C-18 S.P.E. |
| (Oligomeric/ | | | | | Vanillin-Sulfuric Acid |
| Polymeric) | | | | | Assay & n-Butanol- |
| | | | | | HCL assay |
| Anthocyanins | 3.34 | 7.6 | 0.99 | 4.6 | HPLC/MS |
| | | | | | Quantitation (520 nm) |
| | | | | | Cyanidin-3,5- |
| | | | | | Diglucoside Std |
| Catechins | 0.59 | 1.3 | 0.60 | 2.8 | HPLC/MS |
| | | | | | Quantitation (280 nm) |
| | | | | | (+/−)-Catechin Std |
| Phenolic Acids | 1.88 | 4.3 | 1.29 | 6.1 | HPLC/MS |
| | | | | | Quantitation (254 nm) |
| | | | | | Chlorogenic Acid |
| Experimental | 43.84 | 100 | 21.34 | 100 | |
| Total | | | | | |

TABLE 4
Core Muscadine Grape Polyphenols.

Phenolic Acids
Ellagic Acid (aglycone & glycosides)
Gallic Acid
Chlorogenic, Caffeic, Cinnamic, and p-Coumaric
Flavonoids
Anthocyanosides: diglycoside linkages
Anthocyanidins: delphnidin, petunidin, malvidin
Flavonols: quercitin, myricetin, and kaempferol
Flavanols (flavan-3-ol's): catechin, epicatechin
Stilbenes
trans-Resveratrol, piceatannol(tetrahydroxy)
Tannins
Hydrolysable: ellagitannins, gallotannins
Condensed: proanthocyanidins (oligomeric/polymeric)

The extraction methods disclosed above for selectively lowering the levels of condensed tannins, advantageously preserve or improve the levels of other polyphenols that are beneficial to the skin, while avoiding the drawbacks posed by the presence of high levels of condensed tannins.

In certain embodiments, the decolorized muscadine pomace solvent extract is a combination of liquid extracts of bronze and purple muscadine pomace. In some embodiments of the decolorized muscadine pomace solvent extract, the ratio of bronze muscadine pomace extract to purple muscadine pomace extract ranges from about 0.1 to about 10 (weight to weight), such as from about 0.3 to about 3 (weight to weight).

In some examples, the phenolic content of the decolorized muscadine pomace solvent extract includes about 2 to about 3% ellagic acid and about 30 to about 31% gallic acid, for example: about 2 to about 3% ellagic acid, about 3 to about 4% ellagic acid glycosides, about 30 to about 31% gallic acid, about 2 to about 3% quercetin, about 10 to about 11% gallotannins, about 7 to about 8% ellagitannins, about 29 to about 30% proanthocyanidins, about 4 to about 5% anthocyanins, about 2 to about 3% catechins, and about 6 to about 7% phenolic acids.

The purple muscadine pomace extracts used for the decolorized muscadine pomace solvent extract may be comprised of an extract of whole purple (Vitis rotundifolia) muscadine grapes, an extract of purple muscadine pomace from other than whole grapes, or both.

In certain embodiments, the decolorized muscadine pomace solvent extract can include between about 7% to about 10% polyphenols, such as about 9% to about 10% polyphenols, and less than about 5% monosaccharides, such as less than about 4% monosaccharides, by weight of the extract. In a particular example, the decolorized muscadine (Vitis rotundifolia) pomace solvent extract has a polyphenol content of about 9% to about 10%, and contains less than about 4% monosaccharides by weight of the decolored extract.

In some non-limiting examples, the condensed tannins are less than about 10% of the total polyphenol content of the decolorized muscadine pomace solvent extract. In additional non-limiting examples, the decolorized muscadine (Vitis rotundifolia) pomace solvent extract total polyphenols in the decolorized muscadine pomace solvent extract consist of at least about 85%, such as at least about 90%, polyphenols other than condensed tannins.

The decolorized muscadine (Vitis rotundifolia) pomace solvent extract may additionally include naturally occurring components from the grape pomace. For example, the decolorized muscadine pomace solvent extract may contain about 0.5% to about 5% fiber, about 7% to about 14% protein, about 0.5% to about 3% fat, and about 15% to about 20% organic acids by weight of the extract. In certain examples, the decolorized muscadine pomace solvent extract includes about 0.5% to about 2% or about 1% to about 2% fiber, about 7% to about 9% or about 7% to about 8% protein, about 0.05% to about 1% or about 0.5% to about 1.5% fat, and about 15% to about 17% or about 15.5 to about 16.5% organic acids, with each percentage by weight of the extract.

B. Additional Ingredients

Beta-glucans are naturally occurring polysaccharides that contain beta-D-glucose, and are found in the cell walls of certain cereals, yeast, bacteria and fungi. In some examples, the beta-glucan is extracted from a cereal. In particular examples, the beta-glucan is extracted from oat, including the common oat Avena sativa. In certain examples, the beta-glucan has a molecular weight of between about 0.5 to about 1.0×10⁶ Da. The beta-glucan can be a polysaccharide of glucose, and may comprise both 1,4 and 1,3 beta glycosidic linkages.

In some embodiments, beta-glucan provides health benefits, including but not limited to, smoothing skin, moisturizing skin, stimulating fibroblast growth and/or collagen synthesis, reducing the discoloration of scars, and stimulating the immune defense.

Grape seed extract is derived from whole grape seeds and contains beneficial dietary components. Grape seeds are waste products of the winery and grape juice industries. These seeds contain fiber, lipid, protein, carbohydrates, and generally about 4 to about 8% polyphenols (dry weight) depending on the variety. Commercial extracts of grape seeds, including Vitis vinifera grape seed extracts, are often standardized to provide between about 70 to about 95% polyphenolics on a dry weight percentage.

The grape seed extract included in the compositions disclosed herein may be a Vitis vinifera grape seed extract. In some examples, the grape seed extract has a total polyphenol content of less than about 70%, and in some examples the polyphenol content is less than about 50%. In particular examples, the grape seed extract contains about 38 to about 50% polyphenols, about 9 to about 12% fiber, about 1 to about 2% protein, about 25 to about 30% sugars and less than about 1% lipid. In embodiments, the compositions comprise about 0.001% to about 1.0% of decolorized muscadine pomace solvent extract, such as about 0.01% to about 0.5% of decolorized muscadine pomace solvent extract, or about 0.025% to about 0.25% of decolorized muscadine pomace solvent extract, with percentages being based upon the total weight of the composition. In certain examples, the compositions comprise about 0.000001% to about 1.0% beta-glucan, such as about 0.000005% to about 0.1% beta-glucan, or about 0.00005% to about 0.05% beta-glucan, with percentages being based upon the total weight of the topical composition. In some examples, the compositions comprise about 0.0001% to about 1.0% beta-glucan, such as about 0.0001% to about 0.1%, about 0.0005% to about 0.1% beta-glucan, or about 0.00005% to about 0.05% beta-glucan, with percentages being based upon the total weight of the topical composition. In further examples, the compositions comprise about 0.00001% to about 0.1% grape seed extract, such as about 0.0001% to about 0.05% grape seed extract, or about 0.0002% to about 0.01% grape seed extract, with percentages being based upon the total weight of the composition.

Embodiments of the compositions can comprise about 0.001% to about 1.0% of decolorized muscadine pomace solvent extract, 0.000001% to about 1.0% beta-glucan, and about 0.00001% to about 0.1% grape seed extract, with percentages being based upon the weight of the composition. For example, the composition can comprise about 0.01% to about 0.5% of decolorized muscadine pomace solvent extract, about 0.000005% to about 0.1% beta-glucan, and about 0.0001% to about 0.05% grape seed extract, by weight of the composition. In further examples, the composition comprises about 0.025% to about 0.25% of decolorized muscadine pomace solvent extract, about 0.00005% to about 0.05% beta-glucan, and about 0.0002% to about 0.01% grape seed extract, by weight of the composition.

The disclosed compositions can include additional ingredients. Vitamins, such as Vitamin E, have been shown to make the skin softer and smoother after topical application, which can offset some of the damaging effects of the sun and other environmental pollutants. Vitamin A palmitate has been shown to create smoother skin and help enhance the process of cellular turnover. This enhancement rids the skin of the outermost dead layer of skin by bringing more youthful appearing skin cells to the surface. Ingredients that are known to have anti-oxidant activity, such as Vitamin C and superoxide dismutase, or that are believed to be skin rejuvenating ingredients, such as such as lotus japonicus extract and schizandra chinensis fruit extract, may also be included in any of the compositions disclosed herein.

The compositions disclosed herein can also include Vitamin A (retinol and its analogues, such as retinyl palmitate) and/or Vitamin E (tocopherol and its analogues, such as tocopheryl acetate). In certain examples, the compositions comprising a decolorized muscadine pomace solvent extract, beta-glucan and grape seed extract, also include an effective amount of Vitamin A, Vitamin E, or both Vitamins A and E. In certain disclosed embodiments, the compositions comprising a decolorized muscadine pomace solvent extract, beta-glucan and grape seed extract, further include panthenol, Vitamin C (and its analogues, such as magnesium ascorbyl phosphate, ascorbyl palmitate, etc.), and/or superoxide dismutase, which are present in a sufficient amount to improve skin appearance when applied to the skin.

In some embodiments, the composition includes, by weight of the composition, about 0.0001% to about 0.5% Vitamin A, such as about 0.0005% to about 0.01%; about 0.00001% to about 0.1% Vitamin C, such as about 0.0001% to about 0.001%; about 0.001% to about 1.0% Vitamin E, such as about 0.01% to about 0.1%; about 0.0001% to about 1.0% panthenol, such as about 0.0005% to about 0.05%; about 0.00000001% to about 0.1% superoxide dismutase, such as about 0.0000001% to about 0.001%; or any combination thereof. In non-limiting examples of compositions that contain Vitamins C, A and/or E, the Vitamin C may be in the form of magnesium ascorbyl phosphate; the Vitamin A may be in the form of Vitamin A palmitate; and the Vitamin E may be in the form of Vitamin E acetate.

In non-limiting examples of components made with compositions that include extracts of lotus japonicus and/or schizandra chinensis fruit, the composition comprises, by weight of the composition, about 0.01% to about 5.0% lotus japonicus extract, such as about 0.05% to about 0.5%. The compositions can comprise, by weight of the composition, about 0.001% to about 5.0% schizandra chinensis fruit extract, such as about 0.01% or about 0.3%. In an example, the composition comprises, by weight of the composition, about 0.5% lotus japonicus extract and about 0.01% schizandra chinensis fruit extract. In an additional example, the composition comprises, by weight of the composition, about 0.05% lotus japonicus extract and about 0.3% schizandra chinensis fruit extract

In certain examples, the compositions that include panthenol, Vitamins C, A or E, superoxide dismutase, extracts of lotus japonicus and/or schizandra chinensis fruit, or any combination of panthenol, Vitamins C, A and E, superoxide dismutase, lotus japonicus extract, and schizandra chinensis fruit extract, can improve the appearance of skin, when topically applied to the skin.

The CTFA International Cosmetic Ingredient Dictionary and Handbook (2004 and 2008) disclosed a variety of cosmetic ingredients that can be used in the compositions and methods disclosed herein. Any of these cosmetic ingredients can be included in any of the compositions that include a decolorized muscadine pomace solvent extract, as disclosed herein.

The disclosed components can include fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11, and iron oxides), adsorbents, lubricants, solvents, moisturizers (including, e.g., emollients, film formers, and agents that affect the natural moisturizing mechanisms of the skin), water-repellants, UV absorbers (physical and chemical absorbers such as avobenzone, titanium dioxide, zinc oxide, etc.), essential oils, trace metals (e.g., zinc, calcium and selenium), anti-irritants (e.g., steroids and non-steroidal anti-inflammatories), botanical extracts (e.g., aloe vera, chamomile, cucumber extract, ginkgo biloba, ginseng, and rosemary), anti-microbial agents, anti-oxidants (e.g., tocopherol), chelating agents (e.g., sodium phytate and trisodium ethylenediamine disuccinate), preservatives (e.g., phenyoxyethanol, caprylyl glycol and chlorphenesin), pH adjusters (e.g., sodium hydroxide and citric acid), absorbents (e.g., aluminum starch octenylsuccinate, kaolin, corn starch, oat starch, cyclodextrin, and zeolite), skin bleaching and lightening agents (e.g., hydroquinone and niacinamide lactate), humectants (e.g., glycerol, sorbitol, urea, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, and dipotassium glycyrrhizate). Non-limiting examples of some of these ingredients are provided below.

UV Absorption Agents or Sunscreens:

Sunscreens can be included in the moisturizers disclosed herein. A sunscreen alone may be unable to completely protect the skin against the adverse effects of ultraviolet radiation. Any skin care component made with the compositions described herein may additionally include a sunscreen and/or a UV absorption agent. In some embodiments, at least one of the five components further comprises a sunscreen, such as a sunscreen of SPF about 15-50. In an embodiment, the day moisturizer further comprises a sunscreen, which can have a SPF of about 20, about 25, about 30, about 35, about 40, about 45 or about 50.

UV absorption agents that can be used in combination with the compositions of the present invention include chemical and physical sunblocks. Non-limiting examples of chemical sunblocks that can be used include salicylates (homomethyl salicylate, benzyl salicylate, glycol salicylate, isopropylbenzyl salicylate, etc.), anthranilates, ethyl urocanate, homosalate, dibenzoylmethane derivatives (e.g., avobenzone), octocrylene, etc. Non-limiting examples of physical sunblocks include metal oxides (e.g., titanium dioxide and zinc oxide).

Moisturizing Agents:

Moisturizing agents can be included in the cleanser, toner, serum, or moisturizers disclosed herein. Non-limiting examples of moisturizing agents that can be used with any of the compositions and components described herein include amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, propanediol, polyglyceryl sorbitol, salts of pyrollidone carboxylic acid, potassium PCA, sodium glucuronate, sodium PCA, sorbitol, sucrose, trehalose, urea, and xylitol.

Other examples of a moisturizing agent include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamon (elettaria cardamomum) oil, carnauba (copernicia cerifera) wax, carrot (daucus carota sativa) oil, castor (ricinus communis) oil, ceramides, ceresin, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl glucose, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed glycosaminoglycans, hydrolyzed soy protein, hydroxylated lanolin, hydroxyproline, isocetyl stearate, isocetyl stearoyl stearate, isodecyl oleate, isopropyl isostearate, isopropyl lanolate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, methyl gluceth-20, methylsilanol PCA, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, PCA, peach (prunus persica) kernel oil, peanut (arachis hypogaea) oil, pentadecalactone, peppermint (mentha piperita) oil, phospholipids, polyamino sugar condensate, polyglyceryl-3 diisostearate, polyglyceryl-4 caprate, polyquaternium-24, polysorbate 20, polysorbate 40, polysorbate 60, polysorbate 80, polysorbate 85, potassium myristate, potassium palmitate, pyridoxine dipalmitate, retinol, retinyl palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), sodium hyaluronate, sodium lactate, sodium palmitate, sodium PCA, sodium polyglutamate, sodium stearoyl lactylate, sorbitan laurate, sorbitan oleate, sorbitan olivate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sunflower seed wax, sweet almond (prunus amygdalus dulcis) oil, tocopherol, tocopheryl acetate, tocopheryl linoleate, tribehenin, tridecyl neopentanoate, tridecyl stearate, tristearin, urea, vegetable oil, water, waxes, wheat (triticum vulgare) germ oil, and ylang ylang (cananga odorata) oil.

Structuring Agents:

Any of the disclosed compositions can include a structuring agent. Structuring agents, in certain examples, assist in providing rheological characteristics to the composition to contribute to the composition's stability. In other examples, structuring agents can also function as an emulsifier or surfactant. Non-limiting examples of structuring agents include stearic acid, palmitic acid, and mixtures thereof.

Essential Oils:

Essential oils can be included in any of the compositions disclosed herein. Essential oils include oils derived from herbs, flowers, trees, and other plants. Such oils are typically present as tiny droplets between the plant's cells, and can be extracted by several method known to those of skill in the art (e.g., steam distilled, enfleurage (i.e., extraction by using fat), maceration, solvent extraction, or mechanical pressing). When these types of oils are exposed to air, they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to about 240° C. and densities ranging from about 0.759 to about 1.096 g/mL.

Essential oils typically are named by the plant from which the oil is found. For example, rose oil or peppermint oil are derived from rose or peppermint plants, respectively. Non-limiting examples of essential oils that can be used include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present disclosure.

Thickening Agents: Thickening agents can be included in any of the compositions disclosed herein. Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions and components described herein. In certain aspects of the present invention, thickeners include hydrogenated polyisobutene or trihydroxystearin, or a mixture of both.

Non-limiting examples of additional thickening agents that can be used in the context of the present disclosure include carboxylic acid polymers, crosslinked polyacrylate polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich). Non-limiting examples of crosslinked polyacrylate polymers include cationic and nonionic polymers. In an embodiment, the thickener includes an acrylate/C₁₀-C₃₀ alkyl acrylate crosspolymer. In certain embodiments, the thickener includes a copolymer of hydroxyethyl acrylate and sodium acryloyldimethyl taurate. Examples are described in U.S. Pat. Nos. 5,100,660; 4,849,484; 4,835,206; 4,628,078; 4,599,379).

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Other useful polysaccharides include scleroglucans comprising a linear chain of (1-3) linked glucose units with a (1-6) linked glucose every third unit.

Non-limiting examples of gums that can be used with the compositions and components described herein, include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, sclerotium gum, sodium carboyxmethyl dextran, sodium carrageenan, tragacanth gum, xanthan gum, and mixtures thereof.

Preservatives:

Any of the disclosed components can include a preservative. Non-limiting examples of preservatives that can be used in the context of the present disclosure include phenoxyethanol, chlorphenesin, caprylyl glycol, or combinations thereof.

Skin Lightening Agents:

Any of the disclosed components can include a skin lightening agent. Non-limiting examples of skin lightening agents that can be used in the context of the present invention include dipotassium glycyrrhizate, ascorbyl glucoside, niacinamide, licorice extract, or combination thereof.

Pharmaceutical Ingredients:

Any of the disclosed components can include pharmaceutically active agents, although in some embodiments the disclosed components do not include pharmaceutically active agents. Non-limiting examples of pharmaceutical active agents include anti-acne agents, agents used to treat rosacea, analgesics, anesthetics, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, wart treatment agents, wound treatment agents, and wound healing agents.

### III. FORMULATIONS

The decolorized muscadine pomace solvent extract having a reduced content of condensed tannins can be incorporated into a variety of skin care products, such as an oil cleanser, serum concentrate, day moisturizer, clarifying toner, and night skin renewal moisturizer. Each of these can be provided in a separate container. The skin care products can be used in combinations that, when applied to a skin surface, can reduce certain signs of aging skin and improve the appearance of skin. In a non-limiting example, the skin surface is a facial skin surface.

These compositions can be incorporated into all types of cosmetically and dermatologically acceptable vehicles. Non-limiting examples of suitable vehicles include emulsions (e.g., water-in-oil, water-in-oil-in-water, oil-in-water, silicone-in-water, water-in-silicone, oil-in-water-in-oil, oil-in-water-in-silicone emulsions), creams, lotions, solutions (both aqueous and hydro-alcoholic), anhydrous bases (such as lipsticks and powders), gels, and ointments. Additional ingredients can be included, such as, but not limited to, cationic surfactants, occlusive agents, humectants, or silicone containing compounds, or any combination thereof. Exemplary additional ingredients are described above.

Compositions including a decolorized muscadine pomace solvent extract, beta-glucan and grape seed extract, can also contain perfumes, preservatives, dyes, softeners, and physical reflectors, as well as any other class of materials whose presence may be cosmetically or otherwise desirable. The compositions may be in the form of a liquid, gel or semi-solid. The selection of ingredient type and amount is dictated by the nature of the composition, i.e., gel or semi-solid, and is within the skill of cosmetic chemists. For example, larger amounts of oil or wax are incorporated into the semi-solid compositions of the present invention than into the liquid ones.

Kits are also contemplated as being used in certain aspects of the present disclosure. For instance, compositions including a decolorized muscadine pomace solvent extract, beta-glucan and grape seed extract, can be included in a kit. A kit can include one or more containers, each including a component of a combination. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of any of the compositions or component described herein. In other embodiments, the container can be squeezed (e.g., metal, laminate, or plastic tube) to dispense a desired amount of the composition. In certain embodiments, the container can contain fabric pads, such as cotton pads, that are soaked in a composition and used for application of a unit dose to the skin (e.g., toner pads). The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions. The package insert typically includes instructions customarily included in commercial packages of cosmetic products that contain information about the indications, usage, dosage, administration, contraindications and/or warnings concerning the use. The instructional materials may be written, in an electronic form (such as a computer disc or compact disc) or may be visual (such as video files).

A. Cleanser

A cleanser composition is a skin care product that is applied to skin to remove cosmetics, dead skin cells, oil, dirt and other loose material from the skin's surface. A cleanser can be in the form of a liquid, gel or semi-solid. In an example, the cleanser is formulated as a gel, which liquefies upon contact with the skin. A cleanser composition can include water and a detergent, which can unclog pores and remove oil. Detergents, also known as surfactants, can be anionic, cationic, non-ionic or zwitterionic. Cleansers can also include a foaming agent, pH balancing agent, and other pharmaceutically acceptable carriers.

A cleanser composition can, for example, include water, glycerin, surfactants, polymers, preservatives and fragrances. In some embodiments, the cleanser is a composition that includes about 50% to about 90% water, such as about 50%, about 55%, about 60%, about 65%, about 70%, about 80%, or about 85% water, based upon the weight of the cleanser composition. In certain embodiments, the cleanser composition includes about 1% to about 10% surfactant, such as about 2% to about 8%, such as about 2%, about 3%, about 4%, about 5%, about 6%, about 7% or about 8% surfactant by weight of the composition. In an embodiment, the cleanser includes about 1% to about 25% moisturizing agent, such as about 1%, about 5%, about 10%, about 15%, about 20%, or about 25% moisturizing agent by weight of the composition. In further embodiments, the cleanser includes about 1% to about 5% emulsifier, such as about 1%, about 2%, about 3%, about 4%, or about 5% emulsifier by weight of the composition. In additional embodiments, the cleanser includes up to about 1% preservative, such as about 0.1%, about 0.5%, about 0.8% or about 1% by weight of the composition. In an embodiment, the cleanser includes up to about 1% fragrance, such as about 0.1%, about 0.5%, about 0.8% or about 1% by weight of the composition.

In certain embodiments, the cleanser composition comprises about 60% to about 80% water based upon the weight of the cleanser composition, such as about 60%, about 65%, about 70%, about 75% or about 80%. In an embodiment, the cleanser comprises about 1% to about 10% decyl glucose, such as about 1%, about 3%, about 5%, about 7% or about 10%. In certain embodiments, the cleanser comprises about 5% to about 15% glycerin, such as about 5%, about 7%, about 10%, about 12% or about 15%. In further embodiments, the cleanser comprises about 1% to about 10% methyl gluceth-20, such as about 1%, about 3%, about 5%, about 7% or about 10%. In some embodiments, the cleanser comprises about 1% to about 5% polyglyceryl-4 caprate, such as about 1%, about 2%, about 3%, about 4% or about 5%. In additional embodiments, the cleanser comprises about 0.1% to about 1% of an acrylate/C₁₀-C₃₀ alkyl acrylate crosspolymer, such as about 0.1%, about 0.3%, about 0.5%, about 0.8% or about 1%. In yet further embodiments, the cleanser comprises about 0.1% to about 1% fragrance, such as about 0.1%, about 0.3%, about 0.5%, about 0.8% or about 1%. In additional embodiments, the cleanser comprises about 0.1% to about 1% preservative, such as about 0.1%, about 0.3%, about 0.5%, about 0.8% or about 1%. The ingredients can be included in combination.

In some examples, the cleanser composition comprises about 0.001% to about 1.0% of decolorized muscadine pomace solvent extract, about 0.000001% to about 0.1% beta-glucan, and about 0.00001% to about 0.1% grape seed extract, with percentages being based upon the weight of the composition. For example, the cleanser composition comprises about 0.01% to about 0.05% of decolorized muscadine pomace solvent extract, about 0.00001% to about 0.01% beta-glucan, and about 0.0001% to about 0.001% grape seed extract, by weight of the composition. In certain embodiments, the cleanser composition comprises about 0.025% decolorized muscadine pomace solvent extract, about 0.00005% beta-glucan, and about 0.0005% grape seed extract, by weight of the composition. In additional embodiments, the cleanser is a composition that comprises about 0.001% to about 1.0% of decolorized muscadine pomace solvent extract, about 0.0001% to about 0.1% beta-glucan, and about 0.00001% to about 0.1% grape seed extract, with percentages being based upon the weight of the composition.

The grape seed extract used in the cleanser composition can be an aqueous grape seed extract. In an embodiment, the beta-glucan used in the cleanser composition is an aqueous solution.

The cleanser compositions disclosed herein can also include Vitamin A The Vitamin A can be present in the form of retinol and its analogues, such as retinyl palmitate. For example, the cleanser composition comprises between about 0.00005% to about 1.0% Vitamin A, such as between about 0.0001% to about 0.01%. In certain embodiments, the cleanser composition comprises about 0.0005% Vitamin A by weight of the composition.

The cleanser compositions disclosed herein can also include Vitamin E The Vitamin E can be present in the form of tocopherol and its analogues, such as tocopheryl acetate. For example, the cleanser composition comprises between about 0.001% to about 1.0% Vitamin E, such as between about 0.005% to about 0.05%. In certain embodiments, the cleanser composition comprises about 0.01% Vitamin E by weight of the composition.

The cleanser compositions disclosed herein can also include Vitamin C The Vitamin C can be present in the form of ascorbate salts and their analogues, such as magnesium ascorbyl phosphate and ascorbyl palmitate. For example, the cleanser composition comprises between about 0.00001% to about 0.1% Vitamin C, such as between about 0.0005% to about 0.05%. In certain embodiments, the cleanser composition comprises about 0.0001% Vitamin C by weight of the composition.

The cleanser compositions disclosed herein can also include panthenol. For example, the cleanser composition comprises between about 0.0001% to about 1.0% panthenol, such as between about 0.0005% to about 0.05%. In certain embodiments, the cleanser composition comprises about 0.001% panthenol by weight of the composition. In some embodiments, the cleanser composition comprises between about 0.001% to about 1.0% panthenol, such as between about 0.002% to about 0.05%.

The cleanser compositions disclosed herein can additionally include superoxide dismutase. For example, the cleanser composition comprises between about 0.00000001% to about 0.1% superoxide dismutase, such as between about 0.0000005% to about 0.05%. In certain embodiments, the cleanser composition comprises about 0.0000001% superoxide dismutase by weight of the composition. In some embodiments, the cleanser composition comprises between about 0.00001% to about 0.1% superoxide dismutase, such as between about 0.00005% to about 0.01%.

In certain examples, the cleanser compositions comprising a decolorized muscadine pomace solvent extract, beta-glucan and grape seed extract, also include an effective amount of at least one of Vitamin A, Vitamin E, panthenol, Vitamin C, or superoxide dismutase, which are present in a sufficient amount to improve the appearance of skin when applied topically to the skin. In an example, the cleanser composition comprises a decolorized muscadine pomace solvent extract, beta-glucan, grape seed extract, Vitamin A, Vitamin E, panthenol, Vitamin C and superoxide dismutase.

In further embodiments of the disclosed combinations comprising five components, the cleanser composition comprises about 0.01 to about 0.05% or about 0.02 to about 0.04%, such as about 0.02, about 0.025, about 0.03, about 0.035, or about 0.04%, of decolorized muscadine pomace solvent extract by weight of the cleanser composition. In more embodiments, the cleanser composition comprises about 0.00001 to about 0.0001% or about 0.00002 to about 0.00009%, such as about 0.00002, about 0.00003, about 0.00004, about 0.00005, about 0.00006, about 0.00007, about 0.00008, or about 0.00009% beta-glucan by weight. In some embodiments, the cleanser composition comprises about 0.001 to about 0.01% or about 0.002 to about 0.0009%, such as about 0.002, about 0.003, about 0.004, about 0.005, about 0.006, about 0.007, about 0.008, or about 0.009% beta-glucan by weight. In an embodiment, the cleanser composition includes about 0.0001 to about 0.001% or about 0.0002 to about 0.0009%, such as about 0.0002, about 0.0003, about 0.0004, about 0.0005, about 0.0006, about 0.0007, about 0.0008, or about 0.0009% grape seed extract by weight. In additional embodiments, the cleanser composition includes about 0.0001 to about 0.1% or about 0.0002 to about 0.0009%, such as about 0.0002, about 0.0003, about 0.0004, about 0.0005, about 0.0006, about 0.0007, about 0.0008, or about 0.0009% Vitamin A by weight. In further embodiments, the cleanser composition includes about 0.00005 to about 0.01% or about 0.00009 to about 0.0005%, such as about 0.00009, about 0.0001, about 0.0002, about 0.0003, about 0.0004, or about 0.0005% Vitamin C by weight. In yet further embodiments, the cleanser composition includes about 0.005 to about 0.1% or about 0.009 to about 0.05%, such as about 0.009, about 0.01, about 0.02, about 0.03, about 0.04, or about 0.05% Vitamin E by weight. In some embodiments, the cleanser composition includes about 0.0002 to about 0.05% or about 0.0009 to about 0.005%, such as about 0.0009, about 0.001, about 0.002, about 0.003, about 0.004, or about 0.005% panthenol by weight. In still additional embodiments, the cleanser composition includes about 0.00000005 to about 0.00001% or about 0.00000009 to about 0.0000005%, such as about 0.00000009, about 0.0000001, about 0.0000002, about 0.0000003, about 0.0000004, or about 0.0000005% superoxide dismutase by weight. The ingredients can be included in combination. Thus the cleanser composition can include decolorized muscadine pomace solvent extract, beta-glucan, grape seed extract, Vitamin A, Vitamin C, Vitamin E, panthenol, and superoxide dismutase in these concentrations. This cleanser composition can be used in conjunction with a toner composition, day moisturizer composition, serum composition, and/or night moisturizer composition.

B. Toner

A toner composition is a skin care product that is applied to the skin to cleanse the skin and reduce pore size, and may also restore the pH balance in the skin. A toner composition can be in the form of a liquid or gel. In an example, the toner composition is formulated as a liquid that is absorbed into cotton pads, for ease in application to the skin. A toner composition can include water and humectants, as well as alcohols, such as ethanol, an antiseptic agent, an astringent, a soap, lipid, or a surfactant, and other pharmaceutically acceptable carriers. An astringent can constrict skin tissue, resulting in reduced pore size, but can also cause skin dryness. Soaps, lipids and surfactants may be included for their ability to clean the surface of the skin.

A toner composition can, for example, include water, surfactants, moisturizers, alcohols, fragrances and preservatives. In some embodiments, the toner composition includes about 60% to about 99% water, such as about 60%, about 70%, about 80%, about 90%, about 95%, or about 99%, based upon the weight of the toner composition. In certain embodiments, the toner composition includes up to about 1% surfactant, such as about 0.1%, about 0.3%, about 0.5%, about 0.8% or about 1%, by weight of the toner composition. In an embodiment, the toner composition includes about 1% to about 10% moisturizing agent, such as about 1%, about 3%, about 5%, about 8%, or about 10%, by weight of the toner composition. In further embodiments, the toner includes up to about 5% alcohol, such as about 0.5%, about 1%, about 2%, about 3%, about 4%, or about 5%, by weight of the toner composition. In additional embodiments, the toner composition includes up to about 1% preservative, such as about 0.1%, about 0.3%, about 0.5%, about 0.8% or about 1%, by weight of the toner composition. In an embodiment, the toner composition includes up to about 1% fragrance, such as about 0.1%, about 0.3%, about 0.5%, about 0.8% or about 1%, by weight of the toner composition.

In certain embodiments, the toner composition includes about 80% to about 99% water based upon the weight of the toner composition, such as about 80%, about 85%, about 90%, about 95% or about 99%. In an embodiment, the toner composition includes about 1% to about 10% glycerin, such as about 1%, about 3%, about 5%, about 8%, or about 10%. In certain embodiments, the toner composition includes about 0.1% to about 5% alcohol, such as about 0.1%, about 0.5%, about 1%, about 2%, about 3%, about 4%, or about 5%. In further embodiments, the toner composition includes about 0.1% to about 1% polysorbate 20, such as about 0.1%, about 0.3%, about 0.5%, about 0.8% or about 1%. In yet further embodiments, the toner composition includes about 0.1% to about 1% fragrance, such as about 0.1%, about 0.3%, about 0.5%, about 0.8% or about 1%. In an additional embodiment, the toner composition includes about 0.1% to about 1% preservative, such as about 0.1%, about 0.3%, about 0.5%, about 0.8% or about 1%. The ingredients can be included in combination.

In some examples, the toner composition includes about 0.001% to about 1.0% of decolorized muscadine pomace solvent extract, about 0.000001% to about 0.1% beta-glucan, and about 0.00001% to about 0.01% grape seed extract, with percentages being based upon the weight of the composition. For example, the toner composition includes about 0.01% to about 0.05% of decolorized muscadine pomace solvent extract, about 0.00001% to about 0.01% beta-glucan, and about 0.0001% to about 0.001% grape seed extract, by weight of the composition. In certain embodiments, the toner composition includes about 0.025% decolorized muscadine pomace solvent extract, about 0.00005% beta-glucan, and about 0.0005% grape seed extract, by weight of the composition. In an embodiment, the toner composition includes about 0.001% to about 1.0% of decolorized muscadine pomace solvent extract, about 0.0001% to about 0.1% beta-glucan, and about 0.00001% to about 0.01% grape seed extract, with percentages being based upon the weight of the composition.

The grape seed extract used in the toner composition can be an aqueous grape seed extract. In an embodiment, the beta-glucan used in the toner composition is an aqueous solution.

The toner compositions disclosed herein can also include Vitamin A The Vitamin A can be present in the form of retinol and its analogues, such as retinyl palmitate. For example, the toner composition comprises between about 0.00005% to about 1.0% Vitamin A, such as between about 0.0001% to about 0.01%. In certain embodiments, the toner composition comprises about 0.0005% Vitamin A by weight of the composition.

The toner compositions disclosed herein can also include Vitamin E. The Vitamin E can be present in the form of tocopherol and its analogues, such as tocopheryl acetate. For example, the toner composition comprises between about 0.001% to about 1.0% Vitamin E, such as between about 0.005% to about 0.05%. In certain embodiments, the toner composition comprises about 0.01% Vitamin E by weight of the composition.

The toner compositions disclosed herein can also include Vitamin C The Vitamin C can be present in the form of ascorbate salts and their analogues, such as magnesium ascorbyl phosphate and ascorbyl palmitate. For example, the toner composition comprises between about 0.00001% to about 0.1% Vitamin C, such as between about 0.0005% to about 0.05%. In certain embodiments, the toner composition comprises about 0.0001% Vitamin C by weight of the composition.

The toner compositions disclosed herein can also include panthenol. For example, the toner composition comprises between about 0.0001% to about 1.0% panthenol, such as between about 0.0005% to about 0.05%. In certain embodiments, the toner composition comprises about 0.001% panthenol by weight of the composition. In one embodiment, the toner composition comprises between about 0.001% to about 1.0% panthenol, such as between about 0.005% to about 0.05%.

The toner compositions disclosed herein can additionally include superoxide dismutase. For example, the toner composition comprises between about 0.0000001% to about 0.1% superoxide dismutase, such as between about 0.000005% to about 0.05%. In certain embodiments, the toner composition comprises about 0.000001% superoxide dismutase by weight of the composition. In an embodiment, the toner composition comprises between about 0.00001% to about 0.1% superoxide dismutase, such as between about 0.00005% to about 0.01%.

In certain examples, the toner compositions comprising a decolorized muscadine pomace solvent extract, beta-glucan and grape seed extract, also include an effective amount of at least one of Vitamin A, Vitamin E, panthenol, Vitamin C, or superoxide dismutase, which are present in a sufficient amount to improve the appearance of skin when applied topically to the skin. In an example, the toner composition comprises a decolorized muscadine pomace solvent extract, beta-glucan, grape seed extract, Vitamin A, Vitamin E, panthenol, Vitamin C and superoxide dismutase.

In further embodiments of the combination comprising five components, the toner composition includes about 0.01 to about 0.05% or about 0.02 to about 0.04%, such as about 0.02, about 0.025, about 0.03, about 0.035, or 0.04%, of decolorized muscadine pomace solvent extract by weight of the toner composition. In more embodiments, the toner composition includes about 0.00001 to about 0.0001% or about 0.00002 to about 0.00009%, such as about 0.00002, about 0.00003, about 0.00004, about 0.00005, about 0.00006, about 0.00007, about 0.00008, or about 0.00009% beta-glucan by weight. In additional embodiments, the toner composition includes about 0.001 to about 0.01% or about 0.002 to about 0.009%, such as about 0.002, about 0.003, about 0.004, about 0.005, about 0.006, about 0.007, about 0.008, or about 0.009% beta-glucan by weight. In further embodiments, the toner composition includes about 0.0001 to about 0.001% or about 0.0002 to about 0.0009%, such as about 0.0002, about 0.0003, about 0.0004, about 0.0005, about 0.0006, about 0.0007, about 0.0008, or about 0.0009% grape seed extract by weight. In additional embodiments, the toner composition includes about 0.0001 to about 0.1% or about 0.0002 to about 0.0009%, such as about 0.0002, about 0.0003, about 0.0004, about 0.0005, about 0.0006, about 0.0007, about 0.0008, or 0.0009% Vitamin A by weight. In yet other embodiments, the toner composition includes about 0.00005 to about 0.01% or about 0.00009 to about 0.0005%, such as about 0.00009, about 0.0001, about 0.0002, about 0.0003, about 0.0004, or about 0.0005% Vitamin C by weight. In more embodiments, the toner composition includes about 0.005 to about 0.1% or about 0.009 to about 0.05%, such as about 0.009, about 0.01, about 0.02, about 0.03, about 0.04, or about 0.05% Vitamin E by weight. In further embodiments, the toner composition includes about 0.0002 to about 0.05% or about 0.0009 to about 0.005%, such as about 0.0009, about 0.001, about 0.002, about 0.003, about 0.004, or about 0.005% panthenol by weight. In some embodiments, the toner composition includes about 0.0000005 to about 0.0001% or about 0.0000009 to about 0.000005%, such as about 0.0000009, about 0.000001, about 0.000002, about 0.000003, about 0.000004, or about 0.000005% superoxide dismutase by weight. The ingredients can be included in combination. Thus the toner composition can include decolorized muscadine pomace solvent extract, beta-glucan, grape seed extract, Vitamin A, Vitamin C, Vitamin E, panthenol, and superoxide dismutase in these concentrations. This toner can be used in conjunction with a cleanser, day moisturizer, serum, and/or night moisturizer.

C. Day Moisturizer

A moisturizer composition is a skin care product that applied to the skin to add moisture to the external layers of the skin, which can also contain additional ingredients, such as a sunscreen and/or a humectant. A moisturizer composition can be a day moisturizer, which contains a sunscreen. In an embodiment, the day moisturizer composition contains a sunscreen of SPF about 15 to about 50, such as an SPF of about 15, about 20, about 25, about 30, about 35, about 40, about 45 or about 50. In certain non-limiting examples, the day moisturizer composition contains a sunscreen of SPF about 50.

Moisturizers can act on the skin in up to three general ways: by forming a film on the surface of the skin to reduce loss of moisture already in the skin, by attracting water vapor from the air to increase moisture in the skin, or by adding moisturizing factors to the skin surface. A moisturizer composition can be in the form of a liquid, gel or semi-solid. In an example, the moisturizer composition is formulated as an emulsion. Skin moisturizers can make the skin softer and more flexible. The moisturizer composition can include a humectant, lipids, oils, surfactants, and pharmaceutically acceptable carriers. A humectant can increase the moisture level of the skin by both reducing water loss and also by attracting ambient moisture to the skin. Glycerin, urea and collagen are humectants that can be incorporated into a moisturizer composition.

In certain examples, the day moisturizer composition includes water, moisturizing agents, sunscreens, skin conditioners, thickening agents, preservatives and fragrances. In some embodiments, the day moisturizer composition includes about 30% to about 70% water, such as about 30%, about 40%, about 50%, about 60%, or about 70%, based upon the weight of the day moisturizer composition. In certain embodiments, the day moisturizer composition includes about 0.1% to about 30% sunscreen, such as about 0.1%, about 1%, about 5%, about 10%, about 15%, about 20%, about 25%, or about 30%, by weight of the day moisturizer composition. In an embodiment, the day moisturizer composition includes about 1% to about 20% moisturizing agent, such as about 1%, about 5%, about 10%, about 15%, or about 20%, by weight of the day moisturizer composition. In further embodiments, the day moisturizer composition includes about 0.1% to about 6% thickening agent, such as about 0.1%, about 0.5%, about 1%, about 2%, about 3%, about 4%, about 5%, or about 6%, by weight of the day moisturizer composition. In an embodiment, the day moisturizer composition includes up to about 8% skin conditioning agent, such as about 0.5%, about 1%, about 2%, about 3%, about 4%, about 5%, about 6%, about 7%, or about 8%, by weight of the day moisturizer composition. In additional embodiments, the day moisturizer composition includes up to about 2% preservative, such as about 0.1%, about 0.5%, about 1%, about 1.5% or about 2%, by weight of the day moisturizer composition. In an embodiment, the day moisturizer composition includes up to about 1% fragrance, such as about 0.1%, about 0.3%, about 0.5%, about 0.8% or about 1%, by weight of the day moisturizer composition.

In certain embodiments, the day moisturizer composition includes about 40% to about 60% water based upon the weight of the day moisturizer composition, such as about 40%, about 45%, about 50%, about 55%, or about 60%. In an embodiment, the day moisturizer composition comprises about 1% to about 10% glycerin, such as about 1%, about 3%, about 5%, about 8% or about 10%. In certain embodiments, the day moisturizer composition comprises about 5% to about 10% homosalate, such as about 5%, about 6%, about 7%, about 8%, about 9%, or about 10%. In further embodiments, the day moisturizer composition comprises about 1% to about 8% sorbitan olivate, such as about 1%, about 2%, about 3%, about 4%, about 5%, about 6%, about 7%, or about 8%. In some embodiments, the day moisturizer composition comprises about 0.1% to about 5% silica, such as about 0.1%, about 0.5%, about 1%, about 2%, about 3%, about 4%, or about 5%. In additional embodiments, the day moisturizer composition comprises about 0.1% to about 1% of xanthan gum, such as about 0.1%, about 0.3%, about 0.5%, about 0.8% or about 1%. In still additional embodiments, the day moisturizer composition comprises about 5% to about 15% octocrylene, such as about 5%, about 8%, about 10%, about 12%, or about 15%. In certain embodiments, the day moisturizer composition comprises about 0.1% to about 5% avobenzone, such as about 0.1%, about 0.5%, about 1%, about 2%, about 3%, about 4%, or about 5%. In an embodiment, the day moisturizer composition comprises about 0.1% to about 8% dimethicone, such as about 0.1%, about 0.5%, about 1%, about 2%, about 3%, about 4%, about 5%, about 6%, about 7%, or about 8%. In yet further embodiments, the day moisturizer composition comprises about 0.1% to about 1% fragrance, such as about 0.1%, about 0.3%, about 0.5%, about 0.8% or about 1%. In additional embodiments, the day moisturizer comprises about 0.5% to about 2% preservative, such as about 0.5%, about 0.8%, about 1%, about 1.5% or about 2%. The ingredients can be included in combination.

In some examples, the day moisturizer composition includes about 0.001% to about 1.0% of decolorized muscadine pomace solvent extract, about 0.0001% to about 0.1% beta-glucan, and about 0.00001% to about 0.01% grape seed extract, with percentages being based upon the weight of the composition. For example, the day moisturizer composition includes about 0.01% to about 0.05% of decolorized muscadine pomace solvent extract, about 0.001% to about 0.01% beta-glucan, and about 0.0001% to about 0.001% grape seed extract, by weight of the composition. In certain embodiments, the day moisturizer composition includes about 0.025% decolorized muscadine pomace solvent extract, about 0.005% beta-glucan, and about 0.0002% grape seed extract, by weight of the composition.

The grape seed extract used in the day moisturizer composition can be an aqueous grape seed extract. In an embodiment, the beta-glucan used in the day moisturizer composition is an aqueous solution.

The day moisturizer compositions disclosed herein can also include Vitamin E The Vitamin E can be present in the form of tocopherol and its analogues, such as tocopheryl acetate. For example, the day moisturizer composition comprises between about 0.001% to about 1.0% Vitamin E, such as between about 0.005% to about 0.05%. In certain embodiments, the day moisturizer composition comprises about 0.01% Vitamin E by weight of the composition.

The day moisturizer compositions disclosed herein can also include Vitamin C The Vitamin C can be present in the form of ascorbate salts and their analogues, such as magnesium ascorbyl phosphate and ascorbyl palmitate. For example, the day moisturizer composition comprises between about 0.00001% to about 0.1% Vitamin C, such as between about 0.0005% to about 0.05%. In certain embodiments, the day moisturizer composition comprises about 0.0002% Vitamin C by weight of the composition.

The day moisturizer compositions disclosed herein can also include panthenol. For example, the day moisturizer composition comprises between about 0.0001% to about 1.0% panthenol, such as between about 0.0002% to about 0.01%. In an embodiment, the day moisturizer composition comprises between about 0.001% to about 1.0% panthenol, such as between about 0.002% to about 0.5%. In certain embodiments, the day moisturizer composition comprises about 0.0005% panthenol by weight of the composition.

The day moisturizer compositions disclosed herein can additionally include superoxide dismutase. For example, the day moisturizer composition comprises between about 0.00001% to about 0.1% superoxide dismutase, such as between about 0.0005% to about 0.05%. In certain embodiments, the day moisturizer composition comprises about 0.0002% superoxide dismutase by weight of the composition.

In certain examples, the day moisturizer compositions comprising a decolorized muscadine pomace solvent extract, beta-glucan and grape seed extract, also include an effective amount of at least one of Vitamin E, panthenol, Vitamin C, or superoxide dismutase, which are present in a sufficient amount to improve the appearance of skin when applied topically to the skin. In an example, the day moisturizer composition comprises a decolorized muscadine pomace solvent extract, beta-glucan, grape seed extract, Vitamin E, panthenol, Vitamin C, and superoxide dismutase.

In some embodiments of the disclosed combinations comprising five components, the day moisturizer composition includes about 0.01 to about 0.05% or about 0.02 to about 0.04%, such as about 0.02, about 0.025, about 0.03, about 0.035, or about 0.04%, of decolorized muscadine pomace solvent extract by weight of the day moisturizer composition. In certain embodiments, the day moisturizer composition includes about 0.001 to about 0.01% or about 0.002 to about 0.009%, such as about 0.002, about 0.003, about 0.004, about 0.005, about 0.006, about 0.007, about 0.008, or about 0.009% beta-glucan by weight. In some embodiments, the day moisturizer composition includes about 0.0001 to about 0.001% or about 0.0002 to about 0.0009%, such as about 0.0002, about 0.0003, about 0.0004, about 0.0005, about 0.0006, about 0.0007, about 0.0008, or about 0.0009% grape seed extract by weight. In an embodiment, the day moisturizer composition includes about 0.00005 to about 0.01% or about 0.00009 to about 0.0005%, such as about 0.00009, about 0.0001, about 0.0002, about 0.0003, about 0.0004, or about 0.0005% Vitamin C by weight. In additional embodiments, the day moisturizer composition includes about 0.005 to about 0.1% or about 0.009 to about 0.05%, such as about 0.009, about 0.01, about 0.02, about 0.03, about 0.04, or about 0.05% Vitamin E by weight. In further embodiments, the day moisturizer composition includes about 0.0002 to about 0.05% or about 0.0003 to about 0.0007%, such as about 0.0003, about 0.0004, about 0.0005, about 0.0006 or about 0.0007% panthenol by weight. In still additional embodiments, the day moisturizer composition includes about 0.00005 to about 0.01% or about 0.00009 to about 0.0005%, such as about 0.00009, about 0.0001, about 0.0002, about 0.0003, about 0.0004, or about 0.0005% superoxide dismutase by weight. The ingredients can be included in combination. Thus the day moisturizer composition can include decolorized muscadine pomace solvent extract, beta-glucan, grape seed extract, Vitamin C, Vitamin E, panthenol, and superoxide dismutase in these concentrations. This day moisturizer can be used in conjunction with a cleanser, toner, serum, and/or night moisturizer.

D. Night Moisturizer

A night moisturizer composition is a moisturizer composition that is applied to the skin to provide a barrier to protect the skin surface, can act as a vehicle for topical delivery of additional ingredients to the skin, but does not contain a chemical sunscreen. A night moisturizer composition can be in the form of a liquid, gel or semi-solid. In an example, the night moisturizer composition is formulated as a semi-solid emulsion of oil and water. A night moisturizer composition differs from a day moisturizer component, as described herein, in that a night moisturizer composition does not provide sun protection, such as by containing a sunscreen, and is designed to infuse anti-aging ingredients, such as Vitamin A, deep within the skin's surface. Moisturizer compositions, including night moisturizer compositions, can make the skin softer and also form a barrier to retain moisture in the skin. The night moisturizer composition can include a humectant, lipids, oils, surfactants, and pharmaceutically acceptable carriers. Use of a night moisturizer composition can counteract skin dryness due to frequent and/or harsh cleaning. Night moisturizer compositions may also contain glycerol, emulsifying agents and preservatives.

In certain examples, the night moisturizer composition includes water, moisturizing agents, pH adjusters, skin conditioners, thickening agents, preservatives and fragrances. In some embodiments, the night moisturizer composition includes about 40% to about 95% water, such as about 40%, about 50%, about 60%, about 70%, about 80%, about 90%, or about 95%, based upon the weight of the night moisturizer composition. In certain embodiments, the night moisturizer composition includes about 0.1% to about 45% moisturizing agent, such as about 0.1%, about 1%, about 5%, about 10%, about 15%, about 20%, about 25%, about 30%, about 35%, about 40%, or about 45%, based on the weight of the night moisturizer composition. In an embodiment, the night moisturizer composition includes about 0.1% to about 1% pH adjuster, such as about 0.1%, about 0.3%, about 0.5%, about 0.8% or about 1%, based on the weight of the night moisturizer composition. In further embodiments, the night moisturizer composition includes about 0.1% to about 1% thickening agent, such as about 0.1%, about 0.3%, about 0.5%, about 0.8% or about 1%, based on the weight of the night moisturizer composition. In an embodiment, the night moisturizer composition includes about 1% to about 8% skin conditioning agent, based on the weight of the night moisturizer composition. In additional embodiments, the night moisturizer composition includes up to about 1% preservative, such as about 0.1%, about 0.3%, about 0.5%, about 0.8% or about 1%, based on the weight of the night moisturizer composition. In an embodiment, the night moisturizer composition includes up to about 1% fragrance, such as about 0.1%, about 0.3%, about 0.5%, about 0.8% or about 1%, based on the weight of the night moisturizer composition.

In certain embodiments, the night moisturizer composition includes about 50% to about 85% water based upon the weight of the night moisturizer composition, such as about 50%, about 55%, about 60%, about 65%, about 70%, about 75%, about 80%, or about 85%. In an embodiment, the night moisturizer composition includes about 5% to about 15% glycerin, such as about 5%, about 10%, or about 15%. In certain embodiments, the night moisturizer composition includes about 1% to about 10% shea butter, such as about 1%, about 3%, about 5%, about 8% or about 10%. In further embodiments, the night moisturizer composition includes about 1% to about 8% dimethicone, such as about 1%, about 2%, about 3%, about 4%, about 5%, about 6%, about 7% or about 8%. In some embodiments, the night moisturizer composition includes about 0.1% to about 1% sodium hydroxide, such as about 0.1%, about 0.3%, about 0.5%, about 0.8% or about 1%. In additional embodiments, the night moisturizer composition includes about 1% to about 10% sodium stearoyl lactylate, such as about 1%, about 3%, about 5%, about 8% or about 10%. In still additional embodiments, the night moisturizer composition includes about 1% to about 8% caprylic/capric triglycerides, such as about 1%, about 2%, about 3%, about 4%, about 5%, about 6%, about 7% or about 8%. In certain embodiments, the night moisturizer composition includes about 0.1% to about 2% sunflower seed wax, such as about 0.1%, about 0.3%, about 0.5%, about 0.8%, about 1.0%, about 1.5%, or about 2%. In an embodiment, the night moisturizer composition includes about 0.1% to about 1% of an acrylate/C₁₀-C₃₀ alkyl acrylate crosspolymer, such as about 0.1%, about 0.3%, about 0.5%, about 0.8% or about 1%. In yet further embodiments, the night moisturizer composition includes about 0.1% to about 1% fragrance, such as about 0.1%, about 0.3%, about 0.5%, about 0.8% or about 1%. In additional embodiments, the night moisturizer composition includes about 0.1% to about 1% preservative, such as about 0.1%, about 0.3%, about 0.5%, about 0.8% or about 1%. The ingredients can be included in combination.

In some examples, the night moisturizer composition includes about 0.001% to about 1.0% of decolorized muscadine pomace solvent extract, about 0.000001% to about 0.1% beta-glucan, and about 0.00001% to about 0.01% grape seed extract, with percentages being based upon the weight of the composition. For example, the night moisturizer composition includes about 0.01% to about 0.05% of decolorized muscadine pomace solvent extract, about 0.00001% to about 0.01% beta-glucan, and about 0.0001% to about 0.001% grape seed extract, by weight of the composition. In certain embodiments, the night moisturizer composition includes about 0.025% decolorized muscadine pomace solvent extract, about 0.00005% beta-glucan, and about 0.0005% grape seed extract, by weight of the composition. In additional embodiments, the night moisturizer composition includes about 0.025% decolorized muscadine pomace solvent extract, about 0.005% beta-glucan, and about 0.0005% grape seed extract, by weight of the composition.

The grape seed extract used in the night moisturizer composition can be an aqueous grape seed extract. In an embodiment, the beta-glucan used in the night moisturizer composition is an aqueous solution.

The night moisturizer compositions disclosed herein include Vitamin A The Vitamin A can be present in the form of retinol and its analogues, such as retinyl palmitate. For example, the night moisturizer composition comprises between about 0.00005% to about 1.0% Vitamin A, such as between about 0.0001% to about 0.01%. In certain embodiments, the night moisturizer composition comprises about 0.0005% Vitamin A by weight of the composition.

The night moisturizer compositions disclosed herein can also include Vitamin E The Vitamin E can be present in the form of tocopherol and its analogues, such as tocopheryl acetate. For example, the night moisturizer composition comprises between about 0.001% to about 1.0% Vitamin E, such as between about 0.01% to about 0.5%. In certain embodiments, the night moisturizer composition comprises about 0.1% Vitamin E by weight of the composition.

The night moisturizer compositions disclosed herein can also include Vitamin C The Vitamin C can be present in the form of ascorbate salts and their analogues, such as magnesium ascorbyl phosphate and ascorbyl palmitate. For example, the night moisturizer composition comprises between about 0.00001% to about 0.1% Vitamin C, such as between about 0.0005% to about 0.05%. In certain embodiments, the night moisturizer composition comprises about 0.0001% Vitamin C by weight of the composition.

The night moisturizer compositions disclosed herein can also include panthenol. For example, the night moisturizer composition comprises between about 0.001% to about 1.0% panthenol, such as between about 0.005% to about 0.05%. In certain embodiments, the night moisturizer composition comprises about 0.01% panthenol by weight of the composition.

The night moisturizer compositions disclosed herein can additionally include superoxide dismutase. For example, the night moisturizer composition comprises between about 0.0000001% to about 0.1% superoxide dismutase, such as between about 0.000005% to about 0.05%. In an embodiment, the night moisturizer composition comprises between about 0.00001% to about 0.1% superoxide dismutase, such as between about 0.0005% to about 0.05%. In certain embodiments, the night moisturizer composition comprises about 0.000001% superoxide dismutase by weight of the composition.

The night moisturizer compositions disclosed herein can also include an extract of schizandra chinensis fruit. For example, the night moisturizer composition comprises, by weight of the composition, about 0.001% to about 5.0% schizandra chinensis fruit extract. In certain embodiments, the night moisturizer composition comprises about 0.3% schizandra chinensis fruit extract.

In some embodiments, the night moisturizer compositions also includes an extract of lotus japonicus. For example, the night moisturizer composition comprises, by weight of the composition, about 0.001% to about 0.5% lotus japonicus extract. In certain embodiments, the night moisturizer composition comprises about 0.01% to about 0.1%, such as about 0.05%, lotus japonicus extract. In some embodiments, the night moisturizer composition comprises about 0.3% schizandra chinensis fruit extract and about 0.05% lotus japonicus extract.

In certain examples, the night moisturizer compositions that include a decolorized muscadine pomace solvent extract, beta-glucan and grape seed extract, also include an effective amount of at least one of Vitamin A, Vitamin E, panthenol, Vitamin C, superoxide dismutase, schizandra chinensis fruit extract, or lotus japonicus extract, which are present in a sufficient amount to improve the appearance of skin when applied topically to the skin. In an example, the night moisturizer composition comprises a decolorized muscadine pomace solvent extract, beta-glucan, grape seed extract, Vitamin A, Vitamin E, panthenol, Vitamin C, superoxide dismutase, schizandra chinensis fruit extract, and lotus japonicus extract.

In an additional embodiment of the disclosed skin care regimens comprising five components, the night moisturizer composition includes about 0.01 to about 0.05% or about 0.02 to about 0.04%, such as about 0.02, about 0.025, about 0.03, about 0.035, or about 0.04%, of decolorized muscadine pomace solvent extract by weight of the night moisturizer composition. In certain embodiments, the night moisturizer composition includes about 0.00001 to about 0.0001% or about 0.00002 to about 0.00009%, such as about 0.00002, about 0.00003, about 0.00004, about 0.00005, about 0.00006, about 0.00007, about 0.00008, or about 0.00009% beta-glucan by weight. In some embodiments, the night moisturizer composition includes about 0.001 to about 0.01% or about 0.002 to about 0.009%, such as about 0.002, about 0.003, about 0.004, about 0.005, about 0.006, about 0.007, about 0.008, or about 0.009% beta-glucan by weight. In further embodiments, the night moisturizer composition includes about 0.0001 to about 0.001% or about 0.0002 to about 0.0009%, such as about 0.0002, about 0.0003, about 0.0004, about 0.0005, about 0.0006, about 0.0007, about 0.0008, or about 0.0009% grape seed extract by weight. In an embodiment, the night moisturizer composition includes about 0.0001 to about 0.1% or about 0.0002 to about 0.0009%, such as about 0.0002, about 0.0003, about 0.0004, about 0.0005, about 0.0006, about 0.0007, about 0.0008, or about 0.0009% Vitamin A by weight. In additional embodiments, the night moisturizer composition includes about 0.00005 to about 0.01% or about 0.00009 to about 0.0005%, such as about 0.00009, about 0.0001, about 0.0002, about 0.0003, about 0.0004, or about 0.0005% Vitamin C by weight. In yet further embodiments, the night moisturizer composition includes about 0.05 to about 0.5% or about 0.09 to about 0.25%, such as about 0.09, about 0.095, about 0.1, about 0.15, about 0.2, or about 0.25% Vitamin E by weight. In some embodiments, the night moisturizer composition includes about 0.002 to about 0.5% or about 0.009 to about 0.05%, such as about 0.009, about 0.01, about 0.02, about 0.03, about 0.04, or about 0.05% panthenol by weight. In still additional embodiments, the night moisturizer composition includes about 0.0000005 to about 0.0001% or about 0.0000009 to about 0.000005%, such as about 0.0000009, about 0.000001, about 0.000002, about 0.000003, about 0.000004, or about 0.000005% superoxide dismutase by weight. In yet further embodiments, the night moisturizer composition includes about 0.001 to about 1% or about 0.1 to about 0.5%, such as about 0.1, about 0.2, about 0.3, about 0.4, or about 0.5% schizandra chinensis fruit extract by weight. In certain additional embodiments, the night moisturizer composition includes about 0.001 to about 1% or about 0.01 to about 0.05%, such as about 0.01, about 0.02, about 0.03, about 0.04, or about 0.05% lotus japonicus extract by weight. The ingredients can be included in combination. Thus the night moisturizer composition can include decolorized muscadine pomace solvent extract, beta-glucan, grape seed extract, Vitamin A, Vitamin C, Vitamin E, panthenol, superoxide dismutase, schizandra chinensis extract and lotus japonicus extract in these concentrations. This night moisturizer composition can be used in conjunction with a cleanser, toner, serum, and/or day moisturizer.

E. Serum

A serum composition is a skin care product that is applied to the skin to deliver biologically active ingredients deeply into the skin layers. For this reason, a serum composition generally contains higher concentrations of active ingredients. A serum composition can be in the form of a liquid, gel or semi-solid. In an example, the serum composition is formulated as a liquid, with a low viscosity and has a higher proportion of water than oil. In certain embodiments, a serum composition is formulated to penetrate deeply within the skin's surface. A serum composition can include synthetic or natural oils, including poly-alkyl siloxanes, as well as an emulsifying agent, alcohols such as ethanol, a lipid, a pH balancing agent, and other pharmaceutically acceptable carriers. Ingredients that can improve absorption of the serum composition into deeper layers of the skin can also be added, such as pentylene glycol ethanol, dipentaerythrityl hexacaprylate or hexacaprate, and propanediol.

A serum composition can, for example, include water, moisturizing agents, pH adjusters, skin conditioners, thickening agents, surfactants, preservatives and fragrances. In some embodiments, the serum composition includes about 50% to about 95% water, such as about 50%, about 55%, about 60%, about 65%, about 70%, about 75%, about 80%, about 85%, about 90%, or about 95%, based upon the weight of the serum composition. In certain embodiments, the serum composition includes about 2% to about 20% moisturizing agent, such as about 2%, about 5%, about 10%, about 15%, or about 20%, based upon the weight of the serum composition. In an embodiment, the serum composition includes about 0.1% to about 1% pH adjuster, such as about 0.1%, about 0.3%, about 0.5%, about 0.8% or about 1%, based upon the weight of the serum composition. In further embodiments, the serum composition includes about 0.1% to about 4% thickening agent, such as about 0.1%, about 0.5%, about 1%, about 2%, about 3%, or about 4%, based upon the weight of the serum composition. In an embodiment, the serum composition includes about 2% to about 10% skin conditioning agent, such as about 2%, about 3%, about 4%, about 5%, about 6%, about 7%, about 8%, about 9%, or about 10%, based upon the weight of the serum composition. In some embodiments, the serum composition includes about 1% to about 15% surfactant, such as about 1%, about 5%, about 8%, about 10%, about 13%, or about 15%, based upon the weight of the serum composition. In additional embodiments, the serum composition includes up to about 2% preservative, such as about 0.1%, about 0.5%, about 0.8%, about 1%, about 1.3%, about 1.5%, about 1.8% or about 2%, based upon the weight of the serum composition. In an embodiment, the serum composition includes up to about 1% fragrance, such as about 0.1%, about 0.3%, about 0.5%, about 0.8% or about 1%, based upon the weight of the serum composition.

In certain embodiments, the serum composition includes about 60% to about 90% water based upon the weight of the serum composition, such as about 60%, about 70%, about 80%, or about 90%. In an embodiment, the serum comprises about 2% to about 10% glycerin, such as about 2%, about 5%, about 7%, or about 10%. In certain embodiments, the serum comprises about 2% to about 10% polysorbate 20, such as about 2%, about 5%, about 7%, or about 10%. In further embodiments, the serum composition includes about 2% to about 10% dimethicone, such as about 2%, about 5%, about 7%, or about 10%. In some embodiments, the serum composition includes about 0.1% to about 1% sodium hydroxide, such as about 0.1%, about 0.3%, about 0.5%, about 0.8% or about 1%. In additional embodiments, the serum composition includes about 1% to about 3% polysorbate 60, such as about 1%, about 1.5%, about 2%, about 2.5% or about 3%. In still additional embodiments, the serum composition includes about 2% to about 10% propanediol, such as about 2%, about 5%, about 7%, or about 10%. In certain embodiments, the serum composition includes about 0.1% to about 3% of a hydroxyethyl acrylate/sodium acrylolydimethyl taurate copolymer, such as about 0.1%, about 0.5%, about 1%, about 1.5%, about 2%, about 2.5%, or about 3%. In an embodiment, the serum composition includes about 0.1% to about 1% of an acrylate/C₁₀-C₃₀ alkyl acrylate crosspolymer, such as about 0.1%, about 0.3%, about 0.5%, about 0.8% or about 1%. In yet further embodiments, the serum composition includes about 0.1% to about 1% fragrance, such as about 0.1%, about 0.3%, about 0.5%, about 0.8% or about 1%. In additional embodiments, the serum composition includes about 0.5% to about 1.5% preservative, such as about 0.5%, about 0.8%, about 1%, about 1.3% or about 1.5%. The ingredients can be included in combination.

In some examples, the serum composition includes about 0.01% to about 10.0% of decolorized muscadine pomace solvent extract, about 0.00001% to about 1.0% beta-glucan, and about 0.0001% to about 0.1% grape seed extract, with percentages being based upon the weight of the composition. For example, the serum composition includes about 0.1% to about 0.5% of decolorized muscadine pomace solvent extract, about 0.0001% to about 0.1% beta-glucan, and about 0.005% to about 0.1% grape seed extract, by weight of the composition. In certain embodiments, the serum composition includes about 0.25% decolorized muscadine pomace solvent extract, about 0.0005% beta-glucan, and about 0.01% grape seed extract, by weight of the composition. In some embodiments, the serum composition includes about 0.25% decolorized muscadine pomace solvent extract, about 0.05% beta-glucan, and about 0.01% grape seed extract, by weight of the composition

The grape seed extract used in the serum composition can be an aqueous grape seed extract. In an embodiment, the beta-glucan used in the serum composition is an aqueous solution.

The serum compositions disclosed herein can include Vitamin A The Vitamin A can be present in the form of retinol and its analogues, such as retinyl palmitate. For example, the serum composition comprises between about 0.00005% to about 1.0% Vitamin A, such as between about 0.001% to about 0.1%. In certain embodiments, the serum composition comprises about 0.01% Vitamin A by weight of the composition.

The serum compositions disclosed herein can also include Vitamin E The Vitamin E can be present in the form of tocopherol and its analogues, such as tocopheryl acetate. For example, the serum composition comprises between about 0.01% to about 10.0% Vitamin E, such as between about 0.05% to about 5%. In certain embodiments, the serum composition comprises about 0.1% Vitamin E by weight of the composition.

The serum compositions disclosed herein can also include Vitamin C The Vitamin C can be present in the form of ascorbate salts and their analogues, such as magnesium ascorbyl phosphate and ascorbyl palmitate. For example, the serum composition comprises between about 0.0001% to about 1.0% Vitamin C, such as between about 0.0005% to about 0.5%. In certain embodiments, the serum composition comprises about 0.001% Vitamin C by weight of the composition.

The serum compositions disclosed herein can also include panthenol. For example, the serum composition comprises between about 0.001% to about 1.0% panthenol, such as between about 0.005% to about 0.5%. In certain embodiments, the serum composition comprises about 0.05% panthenol by weight of the composition.

The serum compositions disclosed herein can additionally include superoxide dismutase. For example, the serum composition comprises between about 0.000001% to about 1.0% superoxide dismutase, such as between about 0.00005% to about 0.5%. In some embodiments, the serum composition comprises between about 0.0001% to about 1.0% superoxide dismutase, such as between about 0.005% to about 0.5%. In certain embodiments, the serum composition comprises about 0.00001% superoxide dismutase by weight of the composition.

The serum compositions disclosed herein can also include extracts of lotus japonicus and/or schizandra chinensis fruit. For example, the serum composition comprises, by weight of the composition, about 0.001% to about 5.0% schizandra chinensis fruit extract. In certain embodiments, the serum composition comprises about 0.01% schizandra chinensis fruit extract. For example, the serum composition comprises, by weight of the composition, about 0.01% to about 5.0% lotus japonicus extract. In certain embodiments, the serum composition comprises about 0.5% lotus japonicus extract. In some embodiments, the serum composition comprises about 0.01% schizandra chinensis fruit extract and about 0.5% lotus japonicus extract.

In certain examples, the serum compositions comprising a decolorized muscadine pomace solvent extract, beta-glucan and grape seed extract, also include an effective amount of at least one of Vitamin A, Vitamin E, panthenol, Vitamin C, superoxide dismutase, schizandra chinensis fruit extract, or lotus japonicus extract, which are present in a sufficient amount to improve the appearance of skin when applied topically to the skin. In an example, the serum composition comprises a decolorized muscadine pomace solvent extract, beta-glucan, grape seed extract, Vitamin A, Vitamin E, panthenol, Vitamin C, superoxide dismutase, schizandra chinensis fruit extract, and lotus japonicus extract.

In one embodiment of the disclosed combinations comprising five components, the serum component comprises about 0.1 to about 0.5% or about 0.2 to about 0.4%, such as about 0.2, about 0.25, about 0.3, about 0.35, or about 0.4%, of decolorized muscadine pomace solvent extract by weight of the serum composition. In an embodiment, the serum composition includes about 0.0001 to about 0.001% or about 0.0002 to about 0.0009%, such as about 0.0002, about 0.0003, about 0.0004, about 0.0005, about 0.0006, about 0.0007, about 0.0008, or about 0.0009% beta-glucan by weight. In some embodiments, the serum composition includes about 0.01 to about 0.1% or about 0.02 to about 0.09%, such as about 0.02, about 0.03, about 0.04, about 0.05, about 0.06, about 0.07, about 0.08, or about 0.09% beta-glucan by weight. In additional embodiments, the serum composition includes about 0.005 to about 0.05% or about 0.01 to about 0.04%, such as about 0.01, about 0.02, about 0.03, or about 0.04% grape seed extract by weight. In further embodiments, the serum composition includes about 0.0001 to about 0.1% or about 0.005 to about 0.05%, such as about 0.005, about 0.006, about 0.007, about 0.008, about 0.009, about 0.01, about 0.02, about 0.03, about 0.04, or about 0.05% Vitamin A by weight. In further embodiments, the serum composition includes about 0.0005 to about 0.05% or about 0.0009 to about 0.005%, such as about 0.0009, about 0.001, about 0.002, about 0.003, about 0.004, or about 0.005% Vitamin C by weight. In still additional embodiments, the serum composition includes about 0.05 to about 0.5% or about 0.09 to about 0.25%, such as about 0.09, about 0.095, about 0.1, about 0.15, about 0.2, or about 0.25% Vitamin E by weight. In yet further embodiments, the serum composition includes about 0.002 to about 0.5% or about 0.009 to about 0.05%, such as about 0.009, about 0.01, about 0.02, about 0.03, about 0.04, or about 0.05% panthenol by weight. In additional further embodiments, the serum composition includes about 0.000005 to about 0.0005% or about 0.000009 to about 0.00005%, such as about 0.000009, about 0.00001, about 0.00002, about 0.00003, about 0.00004, or about 0.00005% superoxide dismutase by weight. In some embodiments, the serum composition includes about 0.001 to about 1% or about 0.01 to about 0.05%, such as about 0.01, about 0.02, about 0.03, about 0.04, or about 0.05% schizandra chinensis fruit extract by weight. In certain further embodiments, the serum composition includes about 0.01 to about 5% or about 0.1 to about 0.5%, such as about 0.1, about 0.2, about 0.3, about 0.4, or about 0.5% lotus japonicus extract by weight. The ingredients can be included in combination. Thus the serum composition can include decolorized muscadine pomace solvent extract, beta-glucan, grape seed extract, Vitamin A, Vitamin C, Vitamin E, panthenol, superoxide dismutase, schizandra chinensis extract and lotus japonicus extract in these concentrations. This serum composition can be used in conjunction with a cleanser, toner, night moisturizer, and/or day moisturizer.

### IV. SKIN CARE REGIMENS AND METHODS OF IMPROVING THE APPEARANCE OF SKIN

Topical application of one, two, three, four, or all five components disclosed herein can improve the appearance of skin, including improving signs of aging, and benefit overall skin health. Thus, disclosed are method that use a combination of one, two, three, four, or five of the components disclosed herein. Embodiments of skin care methods are presented that include improving the appearance of aging skin by applying to the skin a combination of an effective amount of all five components, the components comprising a toner composition, a day moisturizer composition, a night moisturizer composition, a cleanser composition and a serum composition, to a skin surface having at least one sign of aging. One or more components, such as a combination of two, three, four or all five components, is applied for a period of time sufficient to improve the appearance of the at least one sign of aging. Application of at least one of, or all of, the components can be used to improve skin health, such as to mitigate damage from exposure to the sun or other harmful environmental stimuli, to diminish their damaging effects in the skin. The disclosed methods can reduce the effects of ROS and other environmental exposures, such as to pollution and ultraviolet irradiation, on the skin. Urban dust, which is pollution in a city environment that includes heavy metals, can pose a threat to skin health, such as for people living and working in a city that are exposed to air contaminated with metals. Topical application of one, two, three, four, or all five components disclosed herein can reduce skin damage from urban dust.

In some embodiments, the disclosed methods that include use of one or more compositions comprising decolorized muscadine pomace solvent extract, beta-glucan and grape seed extract to improve skin health, as measured by the assays and analysis presented in the Examples. The disclosed decolorized muscadine pomace solvent extracts, when used in a five-step combination, advantageously preserve or improve the levels of polyphenols with anti-oxidative properties that are beneficial in treatment of the skin.

The disclosed compositions and methods are of use for improving the appearance of skin. The methods include applying an effective amount of one, two, three, four or all five components to the skin. A combination of two, three, four or five components can be applied to the skin. In some embodiments, at least one sign of skin aging is improved. Improvements in the appearance of skin can be determined by analyzing, for example, the depth, length, width, or number of wrinkles present in the skin, as well as skin roughness, sagging, pore size, hyperpigmentation, smoothness, radiance or luminosity, firmness, and the evenness of skin tone. A determination of a benefit to the skin can be evaluated by analysis of these properties, and can include objective physical measurements and subject self-assessment. Physical measurements include, but are not limited to, measurements obtained by instruments such as a cutometer, which measures the extent that skin can be mechanically distended, and imaging systems such as VISIA complexion analysis system.

In some embodiments, the skin care methods include applying the combination including the five components. The compositions that are applied include decolorized muscadine pomace solvent extract, beta-glucan, and grape seed extract; these compositions can improve skin health and skin appearance. The use of the methods disclosed herein can improve intrinsic (such as chronological) and extrinsic (such as environmental) skin damage. Application to the skin of an effective amount of one of more of the disclosed composition for a sufficient period of time provides a measurable improvement in the appearance of the skin.

Skin that shows signs of premature aging can be treated using the disclosed methods. Skin that shows signs of premature aging can be present at any location on the body of a subject. Skin surfaces that are of the most concern generally are those not typically covered by clothing, including facial skin surfaces and neck and chest surfaces. For example, the skin surface can be a facial skin surface including the cheek, forehead, and periorbital surfaces. The periorbital surface includes, but is not limited to, the under eye and crow's feet areas. Any of these can be treated using the disclosed methods.

In some embodiments, facial skin is treated using the disclosed methods. For example, the skin may be in the periorbital, forehead, or cheek area. The skin may be skin that is prematurely aging, in that it shows damage that is normally present in skin that is chronologically older than the age of the subject being treated. In some embodiments, the skin appears to be 5, 6, 7, 8, 9, 10, 11, 12, 13, 14, or 15 years older than the age of the subject. In one non-limiting example, the skin may have environmental or other damage that indicates that it is about 30 years old, whereas the subject may be chronologically only about 25 years old. In some embodiments, the skin is damaged by urban dust.

The components can be applied to skin following a regular treatment schedule, called a regimen. Upon regular and consistent application of one or more of the five components, such as one, two, three, four or all five components, the appearance of the skin is improved. Each component is disclosed in detail above. Any disclosed cleanser, toner, day moisturizer, serum, and night moisturizer can be used in any combination.

In some embodiments, the method includes application of five components comprising decolorized muscadine pomace solvent extract, beta-glucan, and grape seed extract, to the skin. In some embodiments, the methods include periodic application to the skin. In an embodiment, at least one component is applied once every about 24 hours (once daily). In some embodiments, at least one component is applied every about twice daily. The time between applications can vary, and may be about 8, about 9, about 10, about 11, about 12, about 13, about 14, about 15, about 16, about 17, about 18, about 19, about 20, about 21, about 22, about 23, or about 24 hours. In certain embodiments, at least one component is applied once in the morning and another component is applied once in the evening.

In another embodiment, the five components are applied once every about 24 hours (once daily). In some embodiments, the five components are applied once every about twice daily. The time between applications can vary, and may be about 8, about 9, about 10, about 11, about 12, about 13, about 14, about 15, about 16, about 17, about 18, about 19, about 20, about 21, about 22, about 23, or about 24 hours. In certain embodiments, at least one of the five components are applied once in the morning and another one of the five components is applied in the evening.

In any of the skin care methods disclosed herein, not all five components need be applied at every treatment. For example, a subset of the five components may be applied once daily and a subset of the five components may be applied twice daily. In certain embodiments, the cleanser and serum components are each applied twice daily, such as in the morning and the evening, and the day moisturizer, toner and night moisturizer components are each applied once daily, such as the day moisturizer applied only in the morning and the night moisturizer and/or toner applied only in the evening. In another embodiment, the day moisturizer is applied in the morning and the night moisturizer is applied in the evening. In another embodiment, the day moisturizer composition may be applied prior to exposure to the environment and the night moisturizer composition can be applied in the evening.

In some embodiments, the method includes a) applying an effective amount of first application of the cleanser composition the skin in the morning; b) rinsing the first application cleaner composition from the skin; c) applying an effective amount of the day moisturizer composition and an effective amount of the serum composition following step b); d) applying an effective amount of second application of the cleanser composition the skin; e) rinsing the second application of the cleaner composition from the skin; f) applying an effective amount of the toner composition, the serum composition, and an effective amount of the night moisturizer composition to the skin, wherein steps a-c and d-f are separated by about eight to about sixteen hours, such as 8, 9, 10, 11, 12, 13, 14, 15 or 16 hours. Steps a-c can be performed in the morning and steps d-f can be performed in the evening.

In any of the skin care methods disclosed herein, the five components can be applied to the skin in any order. In a further embodiment, the cleanser, serum, and day moisturizer are applied in the morning, and the night moisturizer, the toner, and optionally the serum, are applied in the evening. In one example, the cleanser, serum, toner and day moisturizer are applied at the same time, in that order.

In some embodiments, the cleanser composition is applied first, the skin is rinsed. The cleanser can be used one, two, three, four of five times a day. In some non-limiting examples, the cleanser is used once or twice a day, and then rinsed from the skin. In further non-limiting examples, the cleanser is used in the morning and rinsed from the skin.

In additional embodiments, in the morning, the cleanser composition is applied first, the skin is rinsed. Following rinsing the cleanser composition, the serum composition and the day moisturizer composition are applied, in any order. In the evening, the cleanser composition is applied first, the skin is rinsed. Following rinsing the cleanser composition, the night moisturizer and toner are applied, in any order.

In more embodiments, following rinsing the cleanser composition, the toner composition and serum composition (in any order) are applied. In one non-limiting example, the toner is applied to the skin before the serum. In another non-limiting example, the serum is applied to the skin before the toner. In some embodiments, the day moisturizer composition is applied to the skin following the application of the toner composition and/or serum composition. In some embodiments wherein the day moisturizer is utilized the day moisturizer composition may be applied prior to exposure to the environment (i.e. in the morning).

The application of the night moisturizer composition can be separated from the cleanser, toner, serum and/or day moisturizer composition. For example, the night moisturizer composition can be applied to the skin in the evening, such as at bedtime or dinnertime).

The skin care method can be applied for any length of time wherein at least one sign of aging improves. For example, an effective amount of the five components may be applied to the skin for at least about one day, at least about one week, at least about two weeks, at least about four weeks, at least about eight weeks, or at least about twelve weeks. An effective amount of the five components can be applied for 1, 2, 3, 4, 5, or 6 months. In some embodiments, the five components are applied to the skin surface at least once a day for at least about one week. In additional embodiments, the five components are applied to the skin surface at least twice a day for at least about one week. In further embodiments, the five components are applied to the skin surface at least once a day) for at least about twelve weeks, or are applied to the skin surface at least twice a day for at least about twelve weeks.

In some embodiments of any of the skin care methods presented herein, the improvement of the appearance of aging skin comprises reducing at least one of the depth of wrinkles in the skin, the number of wrinkles in the skin, the length of wrinkles in the skin, or the width of wrinkles in the skin. In certain embodiments, the wrinkles are periorbital wrinkles, forehead wrinkles, or cheek wrinkles. In additional embodiments of any of the skin care methods presented herein, the improvement in the appearance of aging skin comprises at least one of reducing skin roughness, increasing skin smoothness, increasing skin radiance, increasing skin firmness, reducing skin sagging, increasing the evenness of skin tone, reducing pore size, or reducing skin hyperpigmentation.

Improvements to the skin can also include at least one of the following: making facial lines appear less noticeable, making facial lines and/or wrinkles feel plumped, improving the appearance of suborbital lines and/or periorbital lines, improving the appearance of crow's feet, reducing and/or diminishing the appearance of wrinkles, particularly facial wrinkles on the cheeks, forehead (for example, perpendicular wrinkles between eyes, horizontal wrinkles above the eyes), and/or around the mouth, and particularly deep wrinkles, folds, or creases, improving skin suppleness, reducing and/or eliminating fine and/or deep lines, folds and creases, and smoothing skin. Methods for measuring improved skin quality are known in the art. See, for example, U.S. Pat. Nos. 6,866,856 and 6,682,763.

The textural qualities of the skin can be improved, including softness, suppleness, and smoothness, leading to enhancement of luster, clarity and brightness. Additional and important qualities of the skin that can be subjectively and objectively measured include, but are not limited to skin laxity, or conversely skin tightness, and the presence and degree of textural fine lines and coarser lines within the skin.

In an embodiment, the method includes the use of a combination of five components, wherein the components are a toner composition, a day moisturizer composition, a night moisturizer composition, a cleanser composition and a serum composition, is disclosed, wherein each of the components comprise: (i) decolorized muscadine (Vitis rotundifolia) pomace solvent extract comprising a liquid bronze muscadine pomace extract combined with a liquid purple muscadine pomace extract to produce a liquid muscadine pomace extract, wherein a) the bronze muscadine pomace extract and the purple muscadine pomace extract are aqueous extracts; b) the mixture of bronze muscadine pomace extract and purple muscadine pomace extract promotes solubility of ellagic acid in the muscadine pomace extract; c) the bronze muscadine pomace extract and the purple muscadine pomace extract are filtered and fermented extracts; and d) the muscadine pomace extract has a polyphenol content of at least about 2%; (ii) beta-glucan, and (iii) grape seed extract, wherein the appearance of skin is improved upon application of the five components to a skin surface. In some embodiments, the skin surface is a facial skin surface. In certain embodiments each of the toner composition, day moisturizer composition, night moisturizer composition, and cleanser composition comprise about 0.001% to about 1.0% of decolorized muscadine pomace solvent extract by weight of the toner, day moisturizer, night moisturizer, or cleanser composition, about 0.000001% to about 0.1% beta-glucan by weight of the toner composition, day moisturizer composition, night moisturizer composition, or cleanser composition, and about 0.00001% to about 0.01% grape seed extract by weight of the toner composition, day moisturizer composition, night moisturizer composition, or cleanser composition. In additional embodiments, the serum composition comprises about 0.01% to about 10.0% of decolorized muscadine pomace solvent extract by weight of the serum composition, about 0.00001% to about 1.0% beta-glucan by weight of the serum composition, and about 0.0001% to about 0.1% grape seed extract by weight of the serum composition.

In an embodiment, the disclosed methods include the use of a toner composition, day moisturizer composition, night moisturizer composition, and/or cleanser composition, wherein each of the toner composition, day moisturizer composition, night moisturizer composition, and/or cleanser composition, include about 0.01% to about 0.05% of decolorized muscadine pomace solvent extract by weight of the toner, day moisturizer, night moisturizer, or cleanser composition, about 0.00001% to about 0.01% beta-glucan by weight of the toner, day moisturizer, night moisturizer, and/or cleanser composition, and about 0.0001% to about 0.001% grape seed extract by weight of the toner composition, day moisturizer composition, night moisturizer composition, and/or cleanser composition. In a further embodiment, the serum composition comprises about 0.1% to about 0.5% of decolorized muscadine pomace solvent extract by weight of the serum composition, about 0.0001% to about 0.1% beta-glucan by weight of the serum composition, and about 0.005% to about 0.1% grape seed extract by weight of the serum composition.

Any of the components utilized the presently disclosed methods can include an additional ingredient. In some embodiments, at least one of the five components further includes panthenol, Vitamin A, Vitamin C, Vitamin E, superoxide dismutase, lotus japonicus extract, schizandra chinensis fruit extract, or any combination of the aforementioned ingredients. In additional embodiments, all of the five components further includes panthenol, Vitamin A, Vitamin C, Vitamin E, superoxide dismutase, lotus japonicus extract, schizandra chinensis fruit extract, or any combination of the aforementioned ingredients. In certain embodiments, the Vitamin C is in the form of magnesium ascorbyl phosphate, the Vitamin E is in the form of Vitamin E acetate, and the Vitamin A is either in the form of Vitamin A palmitate or retinol.

In some embodiments, at least one of the five components includes, by weight of the composition, about 0.001% to about 1.0% panthenol, about 0.00005% to about 1.0% Vitamin A, about 0.00001 to about 0.1% Vitamin C, about 0.001% to about 1.0% Vitamin E, about 0.00001% to about 0.1% superoxide dismutase, or any combination thereof.

In some embodiments, at least one of the five components further includes lotus japonicus extract, schizandra chinensis fruit extract, or both. In certain embodiments, at least one of the five components includes a sunscreen, which may, for example, have an SPF of about 50. The component can be the day moisturizer.

In one embodiment, the disclosed skincare methods can include the use of combination including a toner, day moisturizer, night moisturizer, cleanser and/or serum component, wherein:
A) the toner composition includes about 0.025% of decolorized muscadine pomace solvent extract by weight of the toner composition; about 0.00005% beta-glucan by weight; about 0.0005% grape seed extract by weight; about 0.0005% Vitamin A by weight; about 0.0001% Vitamin C by weight; about 0.01% Vitamin E by weight; about 0.001% panthenol by weight; and about 0.000001% superoxide dismutase by weight;
B) the day moisturizer composition includes about 0.025% of decolorized muscadine pomace solvent extract by weight of the day moisturizer composition; about 0.005% beta-glucan by weight; about 0.0002% grape seed extract by weight; about 0.0002% Vitamin C by weight; about 0.01% Vitamin E by weight; about 0.0005% panthenol by weight; and about 0.0002% superoxide dismutase by weight;
C) the night moisturizer composition includes about 0.025% of decolorized muscadine pomace solvent extract by weight of the night moisturizer composition; about 0.00005% beta-glucan by weight; about 0.0005% grape seed extract by weight; about 0.0005% Vitamin A by weight; about 0.0001% Vitamin C by weight; about 0.1% Vitamin E by weight; about 0.01% panthenol by weight; about 0.000001% superoxide dismutase by weight; about 0.3% schizandra chinensis fruit extract by weight, and about 0.05% lotus japonicus extract by weight;
D) the cleanser composition includes about 0.025% of decolorized muscadine pomace solvent extract by weight of the cleanser composition; about 0.00005% beta-glucan by weight; about 0.0005% grape seed extract by weight; about 0.0005% Vitamin A by weight; about 0.0001% Vitamin C by weight; about 0.01% Vitamin E by weight; about 0.001% panthenol by weight; and about 0.0000001% superoxide dismutase by weight; and
E) the serum composition includes about 0.25% of decolorized muscadine pomace solvent extract by weight of the serum composition; about 0.0005% beta-glucan by weight; about 0.01% grape seed extract by weight; about 0.01% Vitamin A by weight; about 0.001% Vitamin C by weight; about 0.1% Vitamin E by weight; about 0.05% panthenol by weight; about 0.00001% superoxide dismutase by weight; about 0.01% schizandra chinensis fruit extract by weight, and about 0.5% lotus japonicus extract by weight.

In another embodiment, the disclosed skincare methods can include the use of combination including a toner, day moisturizer, night moisturizer, cleanser and/or serum component, wherein:
A) the toner composition includes about 0.04% of decolorized muscadine pomace solvent extract by weight of the toner composition; about 0.00001% beta-glucan by weight; about 0.0001% grape seed extract by weight; about 0.0001% Vitamin A by weight; about 0.0005% Vitamin C by weight; about 0.05% Vitamin E by weight; about 0.005% panthenol by weight; and about 0.000005% superoxide dismutase by weight;
B) the day moisturizer composition includes about 0.04% of decolorized muscadine pomace solvent extract by weight of the day moisturizer composition; about 0.00001% beta-glucan by weight; about 0.0005% grape seed extract by weight; about 0.0005% Vitamin C by weight; about 0.05% Vitamin E by weight; about 0.0001% panthenol by weight; and about 0.000005% superoxide dismutase by weight;
C) the night moisturizer composition includes about 0.04% of decolorized muscadine pomace solvent extract by weight of the night moisturizer composition; about 0.00001% beta-glucan by weight; about 0.0001% grape seed extract by weight; about 0.0001% Vitamin A by weight; about 0.0005% Vitamin C by weight; about 0.4% Vitamin E by weight; about 0.003% panthenol by weight; about 0.000003% superoxide dismutase by weight; and about 0.5% schizandra chinensis fruit extract by weight;
D) the cleanser composition comprises about 0.04% of decolorized muscadine pomace solvent extract by weight of the cleanser composition; about 0.00001% beta-glucan by weight; about 0.0001% grape seed extract by weight; about 0.0003% Vitamin C by weight; about 0.05% Vitamin E by weight; about 0.003% panthenol by weight; and about 0.000005% superoxide dismutase by weight; and
E) the serum composition includes about 0.4% of decolorized muscadine pomace solvent extract by weight of the serum composition; about 0.0001% beta-glucan by weight; about 0.03% grape seed extract by weight; about 0.05% Vitamin A by weight; about 0.005% Vitamin C by weight; about 0.5% Vitamin E by weight; about 0.001% panthenol by weight; about 0.00003% superoxide dismutase by weight; about 0.05% schizandra chinensis fruit extract by weight, and about 0.3% lotus japonicus extract by weight.

In a further embodiment, the disclosed skincare methods can include the use of combination including a toner, day moisturizer, night moisturizer, cleanser and/or serum component, wherein:
A) the toner composition includes about 0.01% of decolorized muscadine pomace solvent extract by weight of the toner composition; about 0.0003% beta-glucan by weight; about 0.0001% grape seed extract by weight; about 0.0003% Vitamin A by weight; about 0.0005% Vitamin C by weight; about 0.05% Vitamin E by weight; about 0.003% panthenol by weight; and about 0.00005% superoxide dismutase by weight;
B) the day moisturizer composition includes about 0.04% of decolorized muscadine pomace solvent extract by weight of the day moisturizer composition; about 0.0001% beta-glucan by weight; about 0.0005% grape seed extract by weight; about 0.0005% Vitamin C by weight; about 0.0003% panthenol by weight; and about 0.0005% superoxide dismutase by weight;
C) the night moisturizer composition includes about 0.01% of decolorized muscadine pomace solvent extract by weight of the night moisturizer composition; about 0.0003% beta-glucan by weight; about 0.0001% grape seed extract by weight; about 0.0005% Vitamin C by weight; about 0.2% Vitamin E by weight; about 0.0005% panthenol by weight; and about 0.1% schizandra chinensis fruit extract by weight;
D) the cleanser composition includes about 0.01% of decolorized muscadine pomace solvent extract by weight of the cleanser composition; about 0.0003% beta-glucan by weight; about 0.0001% grape seed extract by weight; about 0.0001% Vitamin A by weight; about 0.0005% Vitamin C by weight; about 0.004% panthenol by weight; about 0.0000005% superoxide dismutase by weight, and about 0.1% schizandra chinensis fruit extract by weight; and
E) the serum composition includes about 0.05% of decolorized muscadine pomace solvent extract by weight of the serum composition; about 0.005% beta-glucan by weight; about 0.03% grape seed extract by weight; about 0.05% Vitamin A by weight; about 0.005% Vitamin C by weight; about 0.3% Vitamin E by weight; about 0.002% panthenol by weight; about 0.00005% superoxide dismutase by weight; about 0.03% schizandra chinensis fruit extract by weight, and about 0.4% lotus japonicus extract by weight.

In yet another embodiment, the disclosed skincare methods can include the use of combination including a toner, day moisturizer, night moisturizer, cleanser and/or serum component, wherein:
A) the toner composition includes about 0.025% of decolorized muscadine pomace solvent extract by weight of the toner composition; about 0.005% beta-glucan by weight; about 0.0005% grape seed extract by weight; about 0.0005% Vitamin A by weight; about 0.0001% Vitamin C by weight; about 0.01% Vitamin E by weight; about 0.01% panthenol by weight; and about 0.0001% superoxide dismutase by weight;
B) the day moisturizer composition includes about 0.025% of decolorized muscadine pomace solvent extract by weight of the day moisturizer composition; about 0.005% beta-glucan by weight; about 0.0002% grape seed extract by weight; about 0.0002% Vitamin C by weight; about 0.01% Vitamin E by weight; about 0.005% panthenol by weight; and about 0.0002% superoxide dismutase by weight;
C) the night moisturizer composition includes about 0.025% of decolorized muscadine pomace solvent extract by weight of the night moisturizer composition; about 0.005% beta-glucan by weight; about 0.0005% grape seed extract by weight; about 0.0005% Vitamin A by weight; about 0.0001% Vitamin C by weight; about 0.1% Vitamin E by weight; about 0.01% panthenol by weight; about 0.0001% superoxide dismutase by weight; about 0.3% schizandra chinensis fruit extract by weight, and about 0.05% lotus japonicus extract by weight;
D) the cleanser composition includes about 0.025% of decolorized muscadine pomace solvent extract by weight of the cleanser composition; about 0.005% beta-glucan by weight; about 0.0005% grape seed extract by weight; about 0.0005% Vitamin A by weight; about 0.0001% Vitamin C by weight; about 0.01% Vitamin E by weight; about 0.01% panthenol by weight; and about 0.0001% superoxide dismutase by weight; and
E) the serum composition includes about 0.25% of decolorized muscadine pomace solvent extract by weight of the serum composition; about 0.05% beta-glucan by weight; about 0.01% grape seed extract by weight; about 0.01% Vitamin A by weight; about 0.001% Vitamin C by weight; about 0.1% Vitamin E by weight; about 0.05% panthenol by weight; about 0.001% superoxide dismutase by weight; about 0.01% schizandra chinensis fruit extract by weight, and about 0.5% lotus japonicus extract by weight.

In some embodiments, the decolorized muscadine (Vitis rotundifolia) pomace solvent extract used in the component(s) is prepared as disclosed herein. In some embodiments, the ratio of bronze muscadine pomace extract to purple muscadine pomace extract of the decolorized muscadine pomace solvent extract in each of the five components ranges from about 0.1 to about 10 (weight to weight). In certain embodiments, the ratio of bronze muscadine pomace extract to purple muscadine pomace extract of the decolorized muscadine pomace solvent extract in each of the five components ranges from about 0.3 to about 3 (weight to weight).

In further embodiments, the decolorized muscadine pomace solvent included in the component(s) includes about 7% to about 10% polyphenols and less than about 5% monosaccharides by weight of the decolorized muscadine pomace solvent extract, and the condensed tannins are less than about 10% of the total polyphenol content of the decolorized muscadine pomace solvent extract. In an embodiment, the total polyphenols of the decolorized muscadine pomace solvent extract in each of the five components consist of at least about 85% polyphenols other than condensed tannins. The decolorized muscadine pomace solvent extract included in the components further comprises about 0.5% to about 5% fiber, about 7% to about 14% protein, about 0.05% to about 3% fat and about 15 to about 20% organic acids by weight of the decolorized muscadine pomace solvent extract. In a further embodiment, the decolorized muscadine pomace solvent extract in each of the five components further comprises about 1% to about 2% fiber, about 7% to about 8% protein, about 0.5% to about 1.5% fat and about 15.5% to about 16.5% organic acids by weight of the decolorized muscadine pomace solvent extract.

In still further embodiments, the phenolic content of the decolorized muscadine pomace solvent extract in each of the component(s) comprises about 2 to about 3% ellagic acid and about 30 to about 31% gallic acid by weight of the decolorized muscadine pomace solvent extract. For example, in an embodiment, the phenolic content of the decolorized muscadine pomace solvent extract in each of the five components comprises about 2 to about 3% ellagic acid, about 3 to about 4% ellagic acid glycosides, about 30 to about 31% gallic acid, about 2 to about 3% quercetin, about 10 to about 11% gallotannins, about 7 to about 8% ellagitannins, about 29 to about 30% proanthocyanidins, about 4 to about 5% anthocyanins, about 2 to about 3% catechins, and about 6 to about 7% phenolic acids by weight of the decolorized muscadine pomace solvent extract.

### V. EXAMPLES

Skin care regimens and methods including five components (a toner, a day moisturizer, a night moisturizer, a cleanser and a serum), with each component comprising a composition of decolorized muscadine pomace solvent extract, beta-glucan and grape seed extract, can improve the appearance of skin, as shown by the assays and analytical tests presented in the Examples.

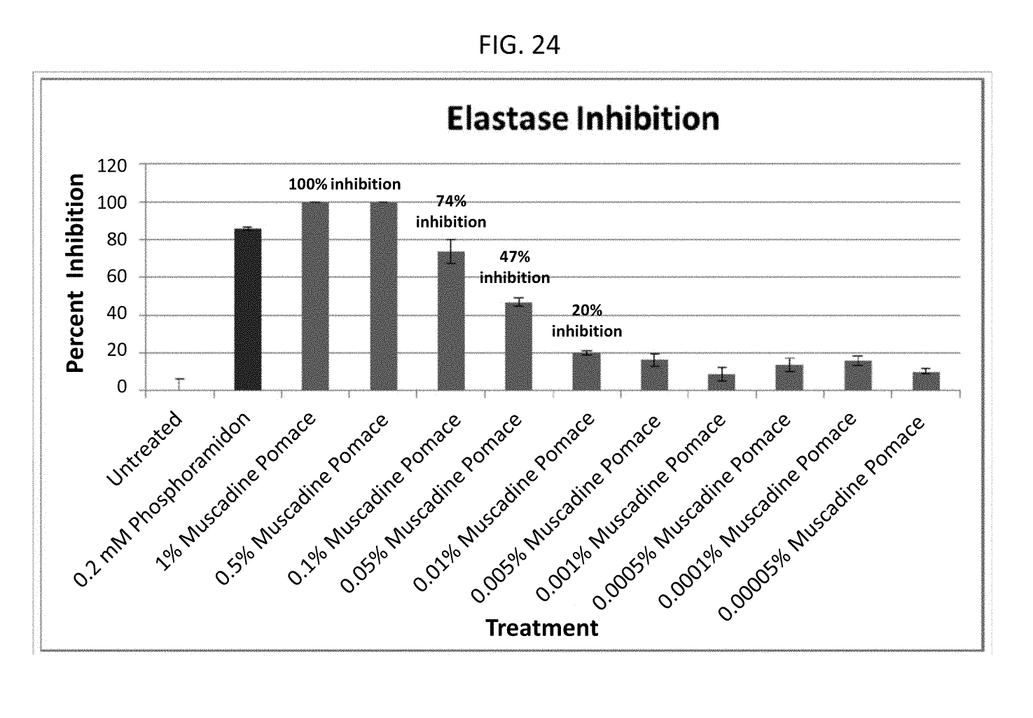

Exemplary results which indicate the mode of action of the decolorized muscadine pomace solvent extract on the skin are presented in Example 1 and FIGS. 24-32. The beneficial effects, including anti-oxidative properties, of compositions containing decolorized muscadine pomace solvent extract are summarized in FIG. 24 for the elastase, collagenase, DPPH, TT dimer, keratinocyte viability, and tyrosinase tests. These tests were performed as described below.

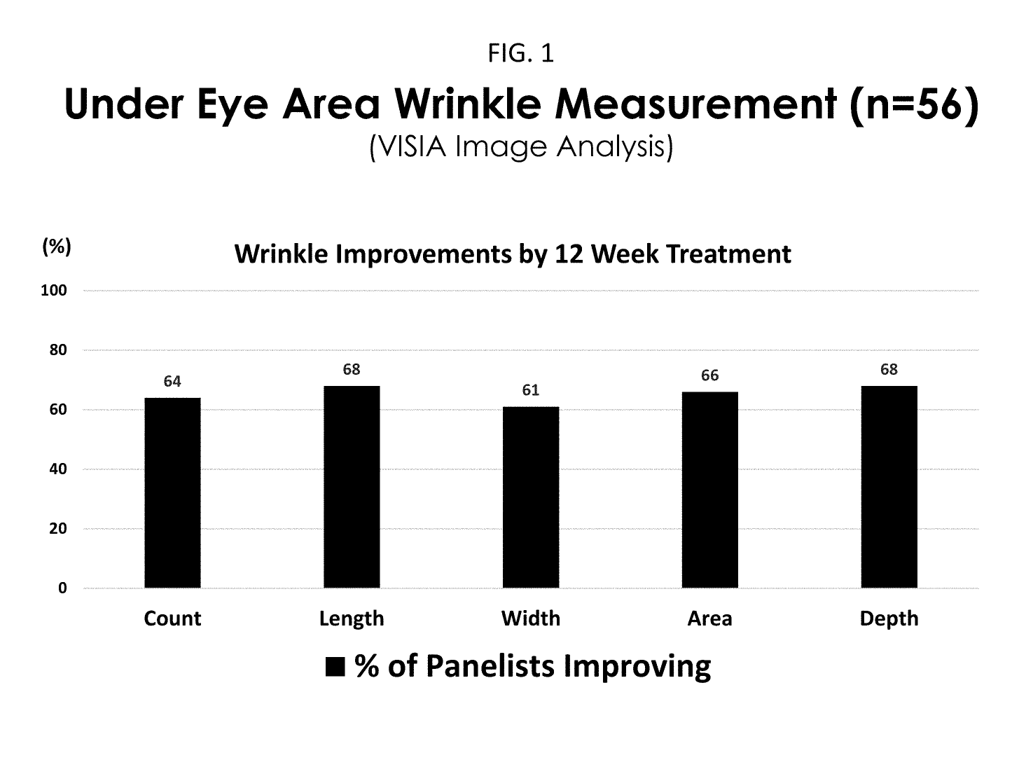

Exemplary results for use of the five components in skin care regimens and methods are presented in Example 2 and FIGS. 1-20. The assays and tests used for the analyses have demonstrated a statistically significant improvement in skin appearance by use of the five components, as evidenced by an evaluation of multiple parameters including the depth, length, width, or number of wrinkles present in the skin, as well as skin roughness, sagging, pore size, hyperpigmentation, smoothness, radiance or luminosity, firmness, and the evenness of skin tone. These parameters were analyzed and/or quantified as described below.

Skin grading parameters include an overall assessment of skin health and appearance, and an analysis of the skin radiance, texture, wrinkling, skin tone/coloration evenness, roughness, pore size and firmness. These parameters were graded on a standardized scale by trained skin graders who assessed skin using a modified Griffiths' 10-point scale according to the following numerical definitions, with half-point scores used as necessary to more accurately describe the skin condition: 0=none (best possible condition); 1 to 3=mild; 4 to 6=moderate; 7 to 9=severe (worst possible condition). The following parameters were evaluated using the indicated scale anchors:

| Parameter | Location(s) | 0 = | 9 = |
|---|---|---|---|
| Wrinkles | Global face | None | Numerous, deep |
| | Periorbital | | wrinkles |
| | Forehead | | |
| | Cheek | | |
| Skin texture | Global face | Smooth, even | Rough, uneven |
| (visual | | looking skin | looking skin texture |
| smoothness) | | texture | |
| Pore size | Global face | Pores are small | Pores are large and |
| | | and not noticeable | prominent |
| Global | Global face | Even skin color, | Significant (severe) |
| hyper- | | no observable | detectable |
| pigmentation | | hyperpigmentation | hyperpigmentation |
| (mottled) | | | appearance, |
| | | | involving most of |
| | | | the face, with very |
| | | | strong intensity |
| Skin tone | Global face | Even, healthy skin | Uneven, discolored |
| (color) | | color | appearance |
| evenness | | | |
| Radiance | Global face | Radiant, luminous | Dull/matte and |
| | | appearance | or/sallow appearance |
| Firmness | Global face | Firm, tight | Loose appearing |
| | | appearing skin | skin |
| Sagging | Global face | None | Visible gravitational |
| | | | looseness |
| Overall | Global face | Excellent, healthy | Poor, unhealthy |
| appearance/ | | looking skin | looking skin |
| healthy look | | | |

For the bioinstrumentation measurements, the following procedures were used. Prior to participating in the following bioinstrumentation measurements, subjects were rested in a designated room within the clinic to acclimate to ambient temperature and humidity conditions for at least 15 minutes. The designated rooms were maintained at a temperature of about 68 to about 75° F. and the relative humidity ranged from about 35 to about 65%. The temperature and humidity of the designated waiting and/or instrumentation rooms were recorded hourly during applicable study visits. If a measurement was unable to be taken, the subject number, location, time point, instrument and reason why, if possible, was recorded as a note to file.

Cutometer Measurements: A single Cutometer measurement was taken on the right ocular bone (directly beneath the center of the eye) at baseline, week 1, week 2, week 4, week 8, and week 12. The Cutometer MPA 580 (Courage+Khazaka, Germany) was used to measure the viscoelastic properties (i.e., firmness and elasticity) of the skin. The instrument applied a vacuum to a small area of skin and measured the elastic response of the skin. The probe was kept at about a 90° angle during the measurements.

A negative pressure of about 300 mbar was applied and released through an about 8-mm probe (standard settings). The measurement lasted for about 30 seconds, during which there were 2 repeated cycles of an about 5-second on (vacuum) time and an about 10-second off (skin release) time. The movement of the skin into and out of the probe was recorded during the application and release of suction. The amount of extensibility, resiliency, pure elasticity and biological elasticity/firmness was recorded.

Chroma Meter Measurements: Chroma Meter measurements were taken on the center of each subject's left cheek (at the intersection of lines extending down from the corner of the eye and horizontally across the bottom of the nose) at baseline, week 1, week 2, week 4, week 8, and week 12. This device took triplicate measurements and produced an average value which was recorded. The Chroma Meter CR-400 (Konica Minolta, Japan) was set to D65 illuminant. The following data was collected by the Chroma Meter, in order to assess skin color: L* values described the relative brightness on a gray scale from black to white; scores increased as the skin tone becomes brighter.

Imaging procedures were described as follows. Prior to photography procedures, clinic personnel ensured that subjects had a clean face with no makeup as described in the study procedures. Subjects removed any jewelry from the areas to be photographed and equilibrated for at least about 15 minutes to ambient conditions within the clinic before any photographs are taken. Subjects were provided with a black or gray matte headband to keep hair away from the face and instructed on proper headband placement. Subjects were provided with a black matte shirt or a black or gray matte cloth that was draped over the subjects' clothing.

VISIA Images: Subjects were instructed to adopt neutral, non-smiling expressions with their eyes gently closed. Subjects were carefully positioned for each photograph, for the center, right, and left side views. Full-face digital images were taken of each subject (right side, left side, and center views) at baseline, week 4, week 8, and week 12, using the VISIA CR photo-station (Canfield Imaging Systems, Fairfield, N.J.) with a Canon Mark II 5D or 6D digital SLR camera (Canon Incorporated, Tokyo, Japan) under the following lighting conditions: Standard lighting 1=visible (bright); Standard lighting 2=visible; Standard lighting 3=raking light for crow's feet area; Cross-polarized; Parallel polarized; or UV spots (blue).

VisioScan Images: A VisioScan VC 98 (Courage+Khazaka electronic GmbH) is a UVA-light (about 340 to about 400 nm) video camera with high resolution that was used to study the skin surface directly. The image captures skin surface texture and dryness. The imaging area was about 6 mm×about 8 mm at a resolution of about 480×about 640 pixels. The VisioScan software was used to calculate and analyze the images for skin roughness on the right cheek. Images were taken at baseline, week 1, week 2, week 4, week 8, and week 12.

### Example 1. Bioassays Demonstrating Beneficial Effects of Decolorized Muscadine Pomace Solvent Extract on Skin

Various bioassays have demonstrated the mode of action of the decolorized muscadine pomace solvent extract on the skin. The beneficial effects of exemplary skin care compositions containing the decolorized muscadine pomace solvent extract for the elastase, collagenase, DPPH, TT dimer, keratinocyte viability, and tyrosinase tests were shown. These tests were performed as follows in a human skin model (MatTek EPIDERM™).

The MatTek EPIDERM™ model uses normal human-derived epidermal keratinocytes that have been cultured to form a multilayered, highly differentiated model of the human epidermis. Ultrastructural analysis has revealed the presence of keratohyalin granules, tonofilament bundles, desmosomes, and a multi-layered stratum corneum containing intercellular lamellar lipid layers arranged in patterns characteristic of in vivo epidermis. Markers of mature epidermis specific differentiation such as pro-filaggrin, the K1/K10 cytokeratin pair, involucrin, and type I epidermal transglutaminase have been localized in this model. The MatTek EPIDERM™ model is also mitotically and metabolically active.

Elastase and Collagenase Inhibition Assay: Human dermal fibroblasts were cultured and used as a source of the elastase enzyme. This enzyme was partially purified from the fibroblasts by lysing the cells in an elastase buffer and retaining the soluble portion of the lysate. Portions of this fibroblast lysate were then be incubated with test materials and a synthetic elastase substrate, Suc-(Ala3)-p-Nitroaniline (STANA). Elastase acts upon this substrate to release p-nitroaniline, which can be detected with a spectrophotometer by measuring the absorbance at a wavelength of about 405 nm. An inhibition of the elastase enzyme is noted by a decrease in the amount of released p-nitroaniline when compared to uninhibited enzyme.

Matrix Metalloproteinase-1 (MMP-1) is an extracellular protease with an approximate molecular weight of about 52 to about 56 kD in its latent form. Upon cleavage of the proenzyme, the about 22 to about 46 kD MMP-1 becomes an active enzyme and can degrade many substrates including collagen, gelatin, and entactin. In human skin, increased MMP-1 activity can be induced via some disease states, exposure to UV irradiation or as part of the natural aging process. This can result an imbalanced state where the degradation of collagen by MMP-1 exceeds its rate of replacement. Therefore, materials that inhibit MMP-1 activity can be beneficial. To screen inhibitors of MMP-1 active human recombinant MMP-1 is incubated in the presence of a thiopeptolide substrate, potential inhibitors and 5,5′-dithiobis(2-nitrobenzoic acid) (DTNB). When the thiopeptolide substrate is broken down by MMP-1 it releases a sulfhydryl group that can react with DTNB and forms 2-nitro-5-thiobenzoic acid, which can be detected spectrophotometrically at about 412 nm. Thus, MMP-1 activity will be proportional to the absorbance at about 412 nm, and in the presence of inhibitors this absorbance will be decreased.

DPPH Assay:

This assay is based on the measurement of the scavenging effect of anti-oxidants on the stable radical 2,2-diphenyl-1-picrylhydrazyl (DPPH). The free radical DPPH has a strong absorbance at about 517 nm, and this absorbance is reduced when DPPH reacts with anti-oxidant compounds and is converted to hydrazine. The DPPH assay is considered a valid and easy assay to evaluate scavenging activity of anti-oxidants, since the radical compound is stable and does not have to be generated as in other radical scavenging assays.

TT Dimer Assay:

The MatTek EPIDERM™ skin model, which consists of normal human-derived epidermal keratinocytes cultured to form a multilayered, highly differentiated model of the human epidermis, was used for this study. The tissues were treated topically either overnight with the test materials prior to UVB exposure (Pretreat group to assess prevention) or treated overnight after a UVB exposure (Post Treat group to assess repair). Following the exposures and treatments, the DNA was extracted from the EPIDERM™ tissues and assayed for thymine dimer content using an ELISA based method.

Ultraviolet-B Radiation Exposure on Keratinocyte Viability:

Human epidermal keratinocytes were treated with the test materials for about 24 hours and then exposed to a dose of UVB light (approximately 50 mj/cm²). Changes in cell viability were then determined about 24 hours post UVB exposure via an MTT assay. The MTT assay is a colorimetric analysis of the metabolic activity of the cell, which is a reflection of cell viability. Viable cells can take up MTT, which is then reduced by mitochondria resulting in the formation of insoluble purple formazin crystals. These crystals are then extracted from the cells with isopropanol and quantified spectrophotometrically. The intensity of the purple color is directly proportional to the number of viable cells and inversely proportional to the toxicity of the test material.

Tyrosinase Inhibition Assay:

Purified tyrosinase enzyme was mixed in a sodium phosphate buffer containing L-DOPA and dbated with the test material. After about 30 minutes of incubation, the amount of L-DOPA converted to DOPA chrome (reflecting tyrosinase activity) is assessed by via a colorimetric assay. Kojic acid is the positive control for tyrosinase inhibition.

Results:

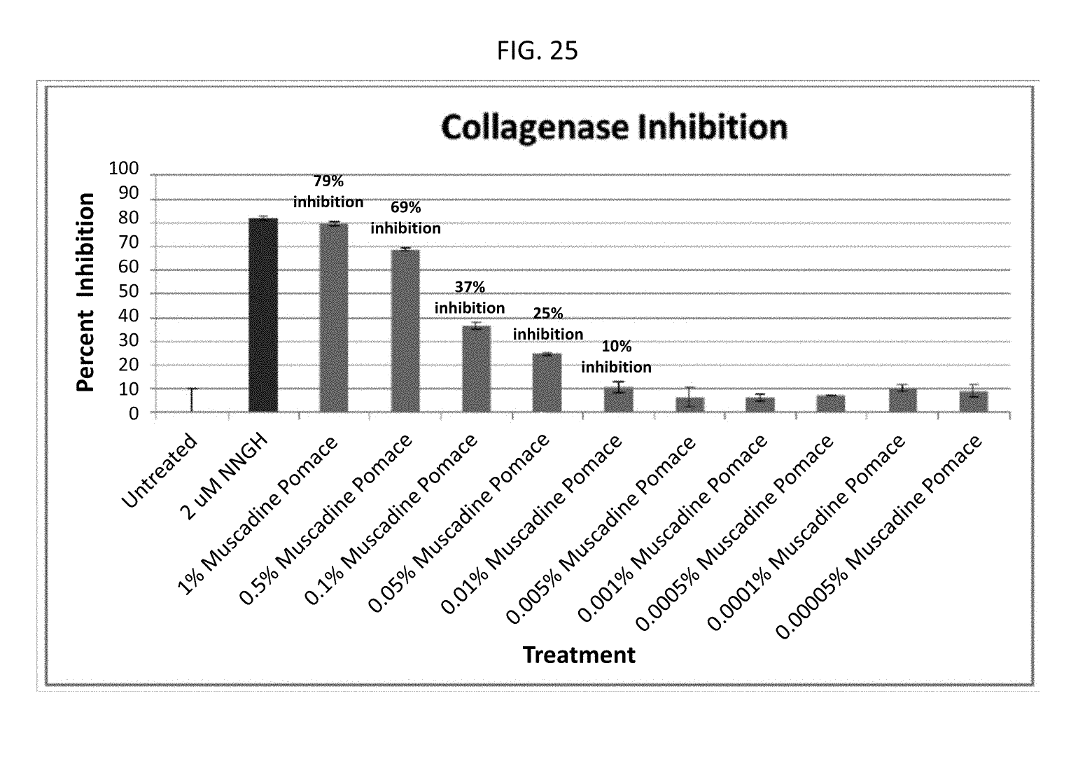

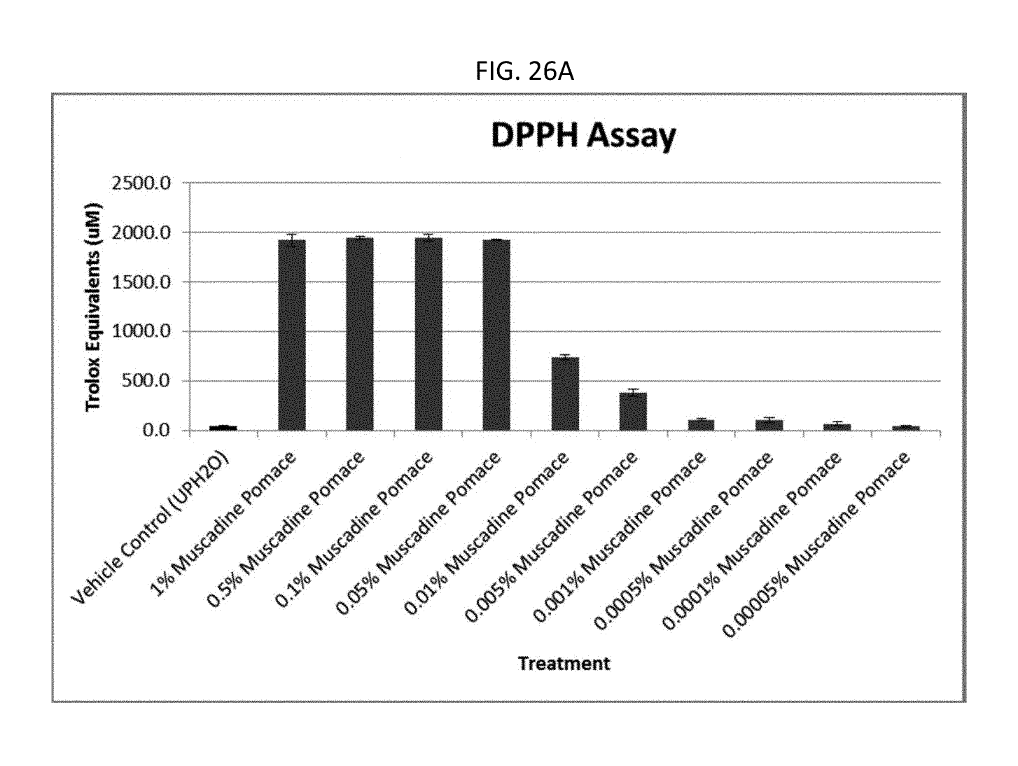

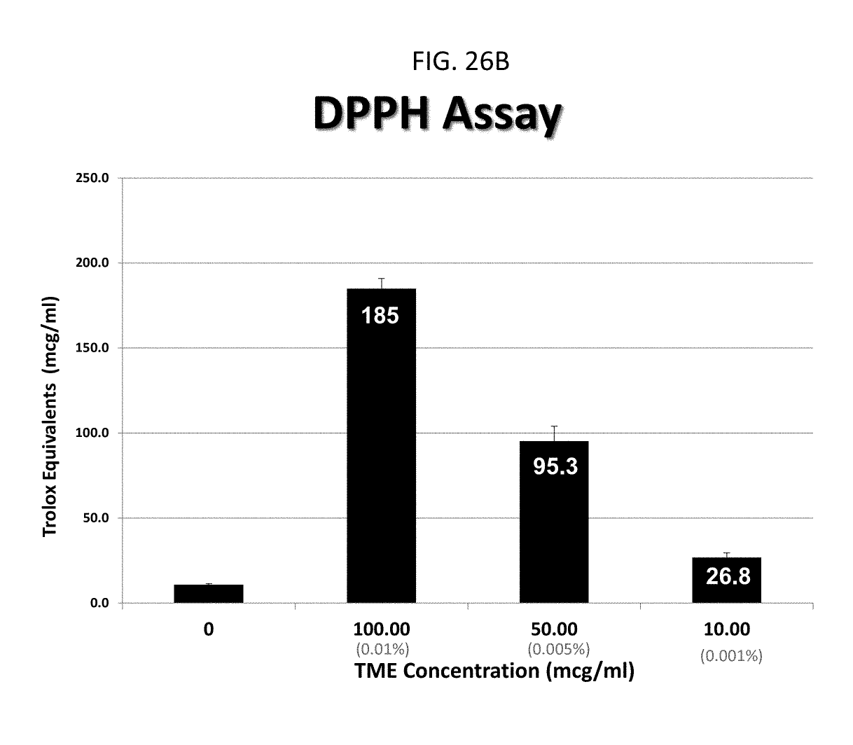

FIG. 24 shows the results of the elastase inhibition test and demonstrates the potential effect on skin elasticity by inhibiting elastin reduction. FIG. 25 shows the results of a collagenase inhibition test that illustrates the potential to maintain skin firmness by avoiding collagen reduction. FIG. 26 shows the results of a DPPH assay that measures free radical scavenging power as shown in Trolox equivalents; the higher the TE value the greater the anti-oxidant power. The decolorized muscadine pomace solvent extract provided anti-oxidant activity greater than about 1500 Trolox equivalents.

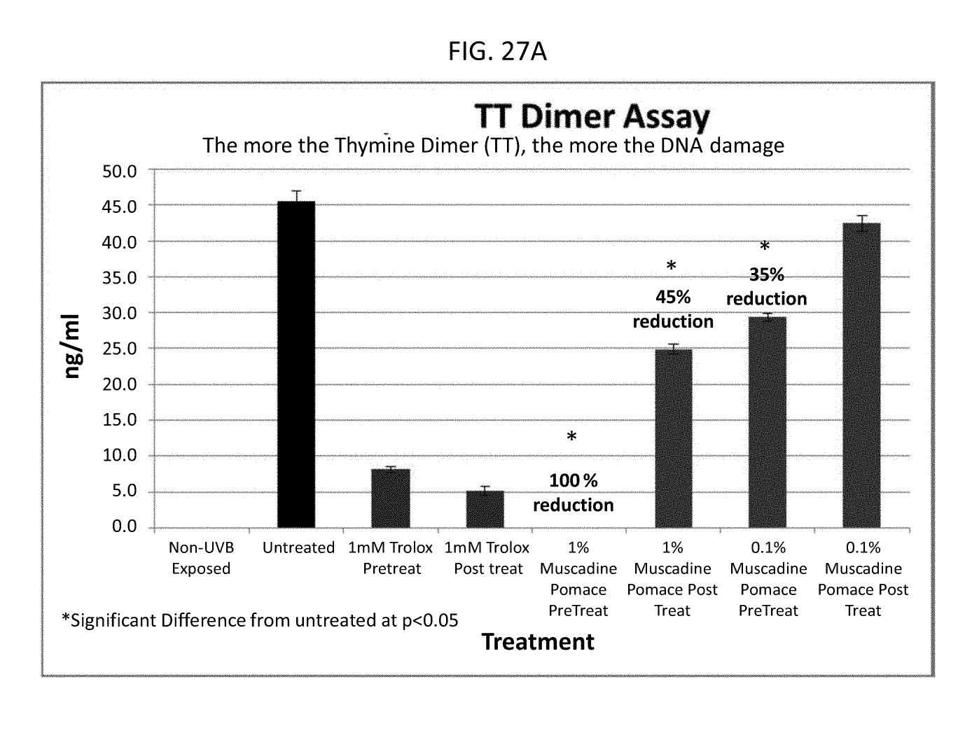

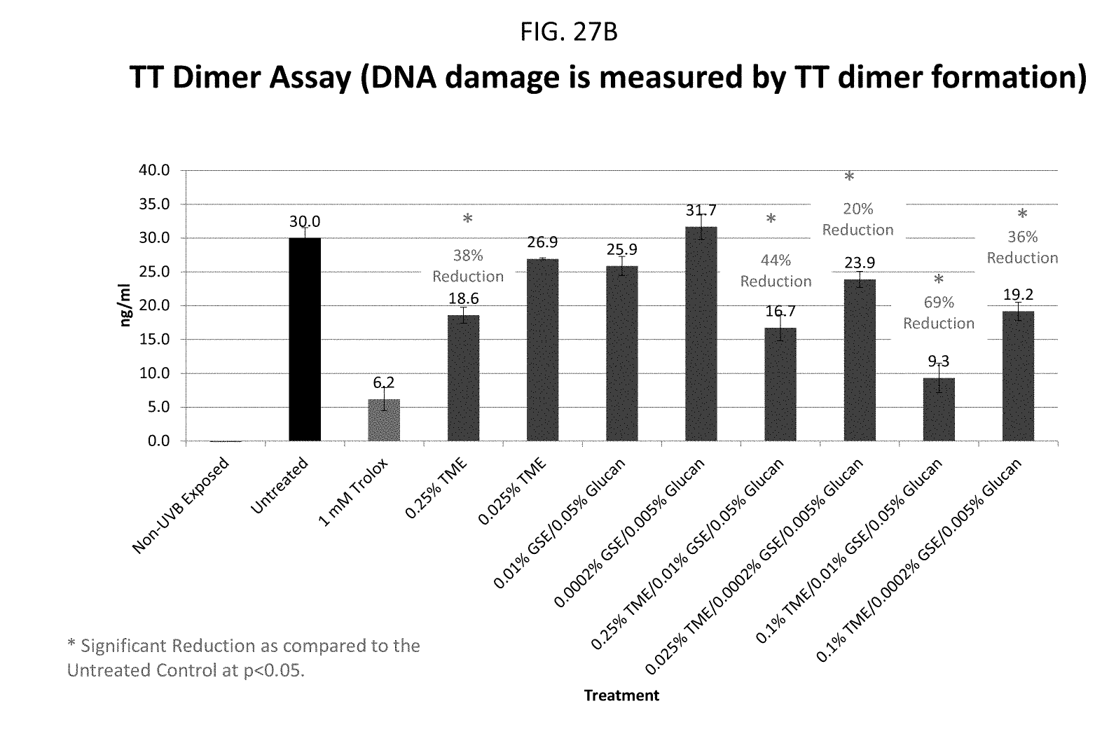

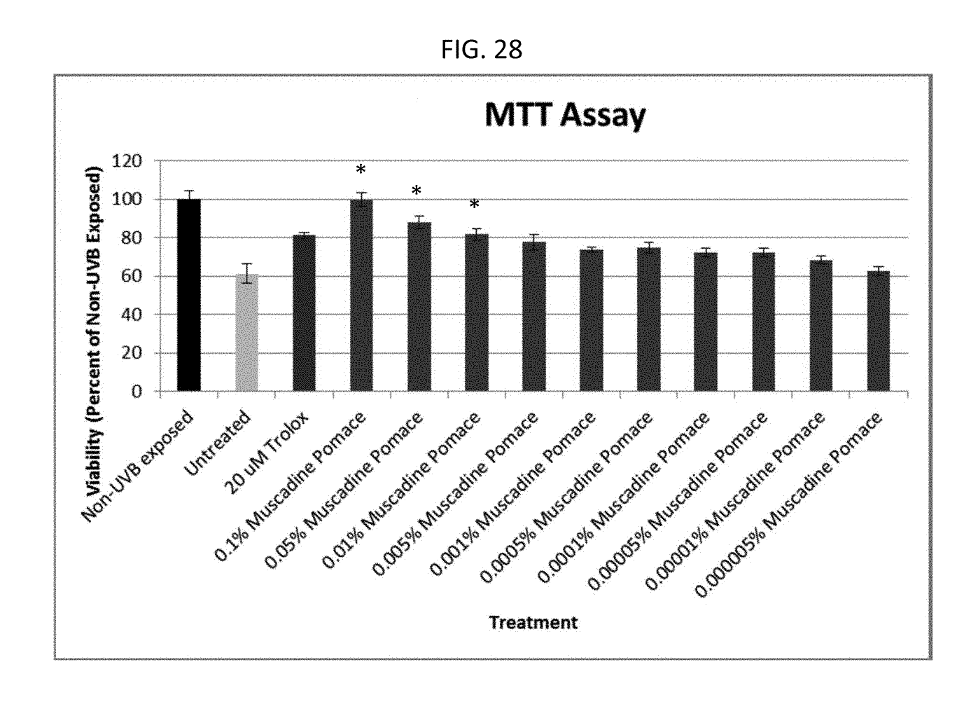

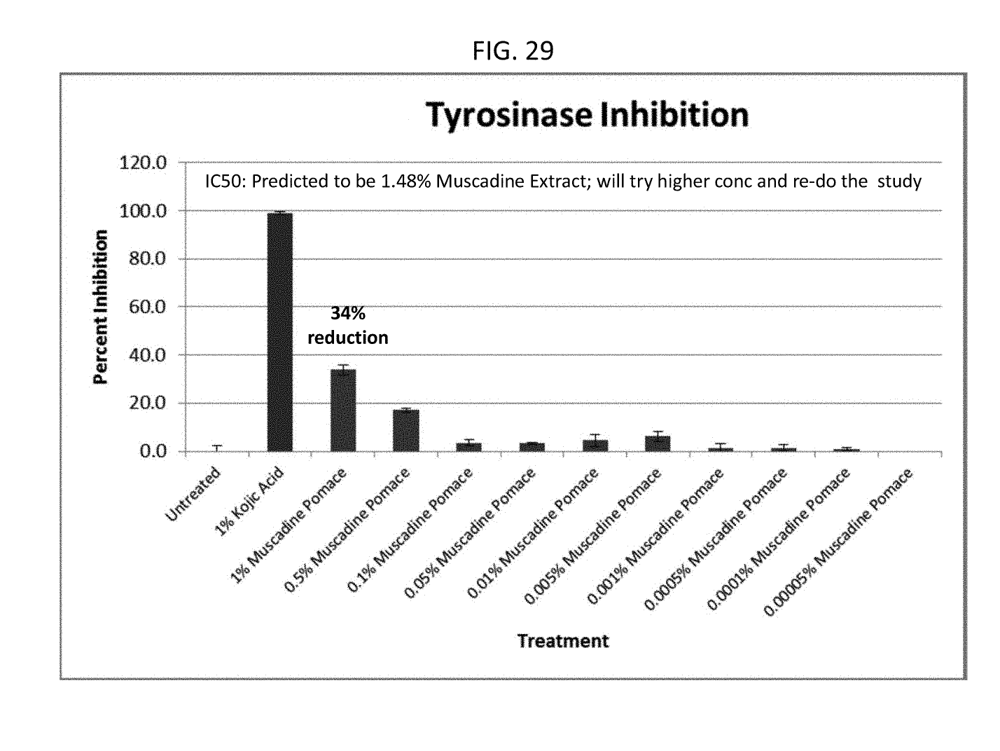

FIG. 27 shows the results of a TT Dimer Assay in which about 1% Muscadine extract pretreatment before UVB exposure completely prevented DNA damage. The decolorized muscadine pomace solvent extract was better than the positive control (1 mM Trolox); about 1% muscadine extract treatment after UVB exposure (post-treatment) also showed about 45% reduction in DNA damage, suggesting an effect on DNA repair; the effect from about 0.1% was lower than about 1% in both pre- and post-treatments. FIG. 28 shows cell survival after ultraviolet B exposure; the decolorized muscadine pomace solvent extracts significantly increased cell survival as compared to untreated cells, and the increase was even better than for about 20 μM Trolox. FIG. 29 show tyrosinase inhibition, which suggests an ability to prevent hyperpigmentation. The decolorized muscadine pomace solvent extract was able to substantially inhibit tyrosinase, and would be expected to help avoid unwanted age- or exposure-related skin pigmentation.

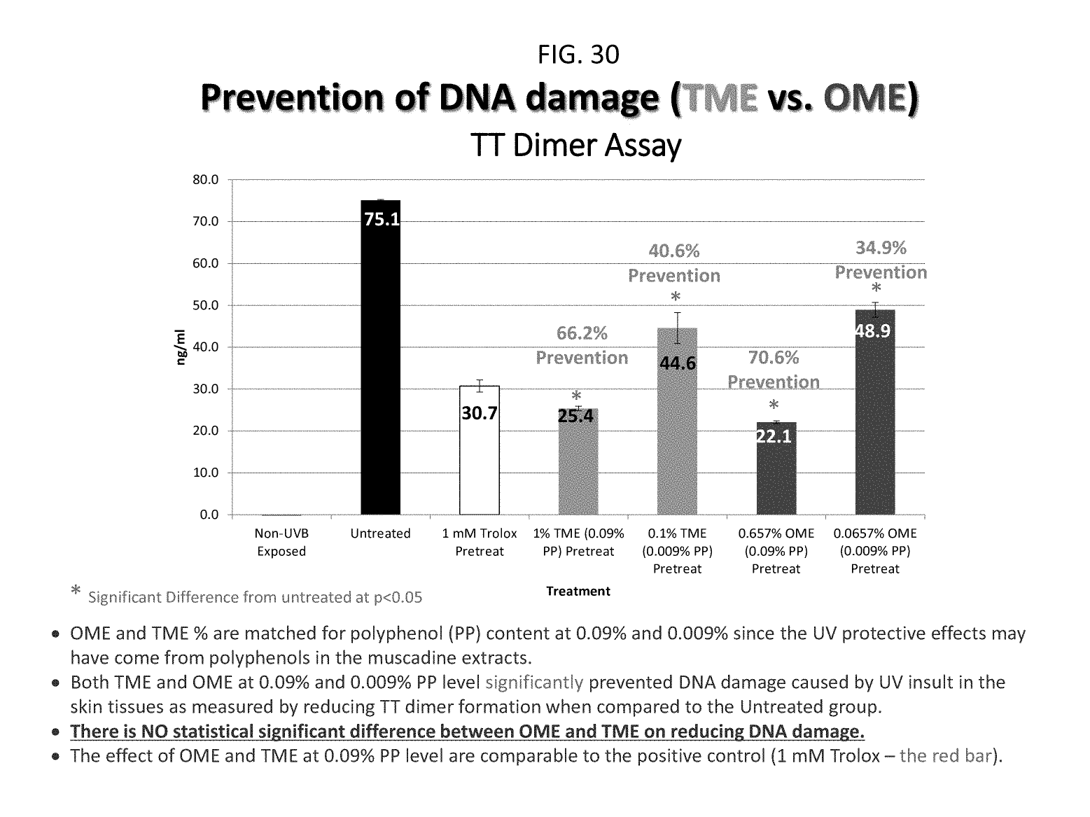

FIG. 30 is a comparative assay that shows the decolorized muscadine pomace solvent extract retains the DNA protectant activity of the precursor extract. FIG. 30 is a graph of a TT Dimer DNA damage comparison assay between a decolorized topical muscadine pomace extract (TME) and orally administered muscadine pomace precursor extract (OME) that has not been decolorized. The OME is the extract disclosed in prior U.S. Pat. No. 8,568,804, U.S. Pat. No. 9,132,162 and U.S. Pat. No. 9,173,916. Both the TME and OME significantly prevented DNA damage caused by UV insult in the skin tissues as measured by reducing TT dimer formation when compared to the untreated group. There was no statistical difference between OME and TME for reducing DNA damage.

In summary, the disclosed decolorized muscadine pomace solvent extracts used at concentrations of about 0.00001% to about 1% by weight, such as, but not limited to about 0.025% to about 0.25% by weight in topical skin care products have lowered levels of condensed tannins and also demonstrate properties that are associated with improved skin elasticity and skin firmness, reduced dark spot formation, improved anti-oxidant activity by free radical scavenging, and protection from ultraviolet-B light, including enhanced skin repair and increased skin cell survival. Decolorization of the extract does not significantly affect the skin protective properties of the extract due to preservation of the overall phenolic profile of the decolorized muscadine pomace solvent extract.

Decolorized Muscadine Pomace Solvent Extract Inhibits Protein Glycation.

Advanced glycation end products (AGEs) are produced by attachment of sugar molecules to cellular and circulating proteins and lipids, a process accelerated by oxidative stress. A variety of AGE molecules have been detected within the body and importantly, increased levels of AGEs are associated with diseases such as diabetes, neurodegeneration, arthritis, and chronic inflammatory disorders. Moreover, a large body of evidence suggests that AGE accumulation underlies the normal process of aging—not only do AGE levels increase with chronological age, but interventions that prolong lifespan (such as caloric restriction) also reduce AGE levels. AGE levels increase in the skin during aging and it has been proposed that glycation of collagen and other skin proteins contributes to the altered appearance and function of aged skin.

Current research suggests that AGEs contribute to cellular aging and dysfunction through two mechanisms. First, the sugars can damage the proteins, interfering with normal function and decreasing cellular viability. Second, AGEs are thought to initiate a vicious cycle of inflammation through their interaction with the receptor for advanced glycation end products (RAGE); activation of RAGE induces multiple inflammatory pathways that ultimately lead to cellular death.

Some, but not all, polyphenols have been shown to inhibit the formation of AGE proteins. It was therefore determined if the decolorized topical muscadine solvent extract (TME) containing reduced tannin levels retained the ability to inhibit protein glycation. Thus, the effects of TME were compared to those of the original/oral muscadine extract (OME). AGE formation was measured using a modification of the standard fluorescence assay as described in Farrar et al., BioFactors 30:193-200, 2007. Briefly, serum albumin is incubated with fructose in phosphate buffer for about 72 hours at about 37° C. and fluorescence intensity is measured at about 370/440 excitation/emission wavelengths. Various concentrations of the muscadine extracts (standardized to μg polyphenols/ml buffer) were compared to control vehicle and results are expressed as percent inhibition of AGE formation produced in the control (vehicle) samples.

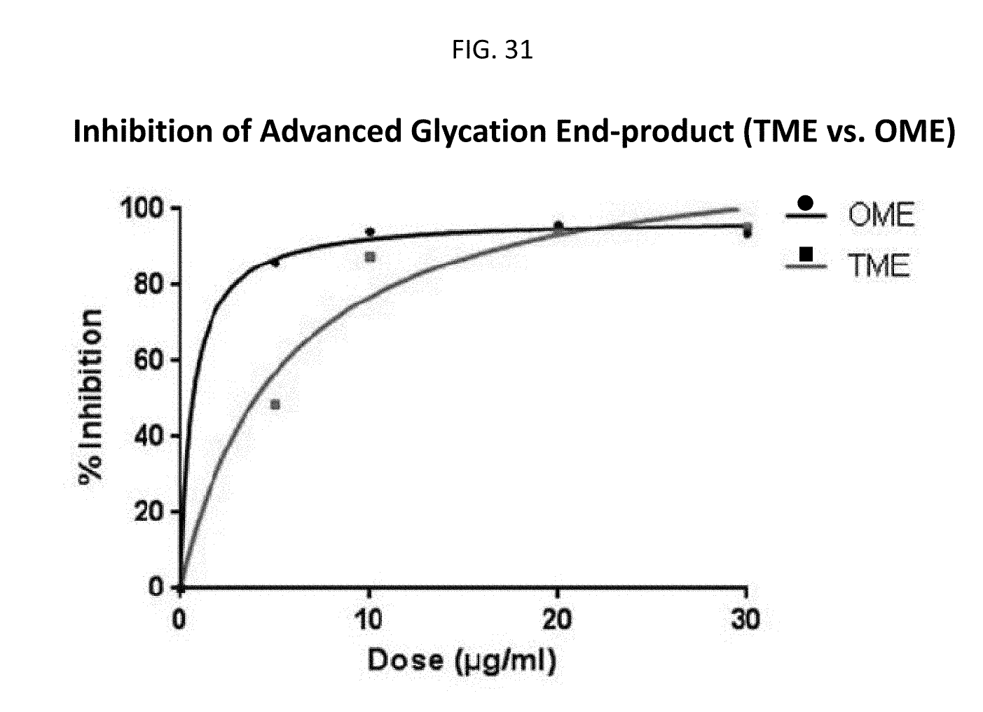

As shown in FIG. 31, both the OME and TME demonstrated excellent activity (efficacy) in inhibiting AGE formation in a dose-related manner Maximal inhibition of AGE formation reached about 95 to about 100% at concentrations of about 15 to about 20 μg polyphenols/ml for both extracts. However, the OME was more potent than the TME as indicated by the concentrations required to inhibit AGE formation by about 50% (IC₅₀) The IC₅₀ value for the OME was about 0.65 μg polyphenols/ml whereas the IC₅₀ value for the TME was about 3.94 μg polyphenols/ml. This suggests that while tannins contribute to the AGE-inhibitory activity, the other muscadine polyphenols remaining in the TME are equally efficacious in inhibiting protein glycation as those found in the OME.

### Example 2. Efficacy Study of Skin Care Regimens and Methods Using Compositions Comprising Decolorized Muscadine Pomace Solvent Extract

A study with human subjects was performed to evaluate the efficacy of five components for skin care, with each component comprising a combination of decolorized muscadine pomace solvent extract, beta-glucan, and grape seed extract. The study involved 57 women (28 Caucasian women and 29 Asian women), ranging in age from about 30 to about 65 years old. Each subject followed a skin care treatment schedule using cleanser, toner pads, serum, day moisturizer and night moisturizer skin care products containing ranges of ingredients as described in Tables 5A and 5B, for a total of twelve weeks. Each subject was evaluated for skin health at the beginning of the study (i.e. week 0), and after one, two, four, eight and twelve weeks, by trained skin graders.

TABLE 5A
Ingredient ranges used in the compositions of the five components.¹

| | | day | night | | |
|---|---|---|---|---|---|
| | toner | moisturizer | moisturizer | cleanser | serum |
| TME² | 0.001-1% | 0.001-1% | 0.001-1% | 0.001-1% | 0.01-10% |
| beta-glucan | 0.000001-0.1% | 0.0001-0.1% | 0.000001-0.1% | 0.000001-0.1% | 0.00001-1% |
| grape seed | 0.00001-0.01% | 0.00001-0.01% | 0.00001-0.01% | 0.00001-0.01% | 0.0001-0.1% |
| extract | | | | | |
| Vitamin A³ | 0.00005-1% | 0-1% | 0.00005-1% | 0.00005-1% | 0.0005-1% |
| Vitamin C⁴ | 0.00001-0.1% | 0.00001-0.1% | 0.00001-0.1% | 0.00001-0.1% | 0.0001-0.1% |
| Vitamin E⁵ | 0.001-1% | 0.001-1% | 0.001-1% | 0.001-1% | 0.01-1% |
| panthenol | 0.0001-1% | 0.0001-1% | 0.001-1% | 0.0001-1% | 0.001-1% |
| superoxide | 0.0000001-0.1% | 0.00001-0.1% | 0.0000001-0.1% | 0.00000001-0.1% | 0.000001-0.1% |
| dismutase | | | | | |
| schizandra | 0-10% | 0-10% | 0-10% | 0-10% | 0-10% |
| chinensis | | | | | |
| fruit extract | | | | | |
| lotus | 0-10% | 0-10% | 0-10% | 0-10% | 0-10% |
| japonicus | | | | | |
| extract | | | | | |

¹Percentages shown are as a weight percentage of the weight of the total composition.
²TME is the disclosed decolorized topical muscadine solvent extract.
³As retinyl palmitate.
⁴As magnesium ascorbyl phosphate.
⁵As tocopherol acetate.

TABLE 5B
Additional ingredient ranges used in the compositions of the five components.¹

| | | day | night | | |
|---|---|---|---|---|---|
| | toner | moisturizer | moisturizer | cleanser | serum |
| TME² | 0.01-0.05% | 0.01-0.05% | 0.01-0.05% | 0.01-0.05% | 0.1-0.5% |
| beta-glucan | 0.000001-0.01% | 0.0001-0.01% | 0.000001-0.01% | 0.000001-0.01% | 0.00001-0.1% |
| grape seed | 0.0001-0.001% | 0.0001-0.001% | 0.0001-0.001% | 0.0001-0.001% | 0.005-0.1% |
| extract | | | | | |
| Vitamin A³ | 0.0001-0.1% | none | 0.0001-0.1% | 0.0001-0.1% | 0.005-0.1% |
| Vitamin C⁴ | 0.00005-0.01% | 0.00005-0.01% | 0.00005-0.01% | 0.00005-0.01% | 0.0005-0.05% |
| Vitamin E⁵ | 0.005-0.1% | 0.005-0.1% | 0.05-0.5% | 0.005-0.1% | 0.05-0.5% |
| panthenol | 0.0001-0.5% | 0.0001-0.5% | 0.002-0.5% | 0.0001-0.5% | 0.002-0.5% |
| superoxide | 0.0000001-0.01% | 0.00005-0.01% | 0.0000001-0.01% | 0.00000001-0.01% | 0.000001-0.05% |
| dismutase | | | | | |
| schizandra | 0-1% | 0-1% | 0.001-1% | 0-1% | 0.001-1% |
| chinensis | | | | | |
| fruit extract | | | | | |
| lotus | 0-1% | 0-1% | 0.001-1% | 0-1% | 0.01-5% |
| japonicus | | | | | |
| extract | | | | | |

¹Percentages shown are as a weight percentage of the weight of the total composition.
²TME is the disclosed decolorized topical muscadine solvent extract.
³As retinyl palmitate.
⁴As magnesium ascorbyl phosphate.
⁵As tocopherol acetate.

In the study, each subject used the cleanser, serum and day moisturizer products in the morning, and the cleanser, toner pads, serum and night moisturizer products in the evening. At the end of the end of the twelve weeks, each subject filled out a self-assessment questionnaire on their perceived skin health. The regimen was well tolerated by the subjects during the twelve week study.

Trained personnel evaluated the skin health of the subjects on ten graded parameters, listed in Table 6, below. Four skin health parameters were evaluated using instrumentation: melanin levels, resilience, elasticity, and skin roughness. The results of the study indicate that some skin health parameters showed a statistically significant improvement within as little as one week of treatment.

TABLE 6
Parameters evaluated to determine skin health.

| Graded Parameters | Instrumentation Parameters |
|---|---|
| cheek wrinkles | chromameter b: melanin production |
| forehead wrinkles | cutometer R2: resilience |
| periorbital wrinkles | cutometer R7: biological elasticity |
| | and firmness |
| skin texture | VisioScan R1: roughness depth |
| pore size | VISIA Image: wrinkles in under eye |
| | and crow's feet areas |
| global hyperpigmentation | |
| skin tone evenness | |
| radiance | |
| firmness | |
| sagging | |

The instrumentation results on roughness indicated that all skin roughness parameters (roughness depth, maximum roughness, average roughness and skin smoothness) showed statistically significant improvements after two weeks of treatment which continuously improved until the end of the study (12 weeks), indicating smoother skin. The wrinkle image analysis showed statistically significant improvements after four weeks of treatment, lasting until the end of the study (twelve weeks) for the under eye area (wrinkle count, length, width, area and depth). Data obtained with the cutometer showed statistically significant improvements after eight weeks of treatment in skin extensibility (firming).

The graded parameter results indicate that statistically significant improvements in many parameters occurred starting after one or two weeks of treatment and continuing through the end of the study (twelve weeks). The skin texture, radiance and overall health parameters showed an improvement in one week. The global face wrinkle, periorbital wrinkle, forehead wrinkle, cheek wrinkle, pore size, skin tone (color) evenness, firmness and roughness parameters improved after two weeks of treatment. The global hyperpigmentation parameter improved after four weeks of treatment. The sagging and firmness parameters improved after eight weeks of treatment. The under eye and crow's feet parameters improved after twelve weeks of treatment. The results of the efficacy study are summarized in Table 7, below.

TABLE 7
Parameters showing statistically significant improvement vs.
treatment time.

| | time to first significant |
|---|---|
| parameters | improvement |
| skin texture (visual smoothness) | 1 week |
| radiance | 1 week |
| overall appearance/healthy global face look | 1 week |
| global face wrinkles (overall face) | 2 weeks |
| periorbital wrinkles | 2 weeks |
| forehead wrinkles | 2 weeks |
| cheek wrinkles | 2 weeks |
| pore size | 2 weeks |
| skin tone (color) evenness | 2 weeks |
| firmness | 2 weeks |
| roughness (improved smoothness) | 2 weeks |
| global hyperpigmentation | 4 weeks |
| sagging | 8 weeks |
| firmness (extensibility) | 8 weeks (by cutometer)* |
| wrinkles in under eye area | 12 weeks (by VISIA) |
| wrinkles in crow's feet area | 12 weeks (by VISIA) |

*Firmness (extensibility) showed a statistically significant improvement only after eight weeks of treatment, but not after twelve weeks.

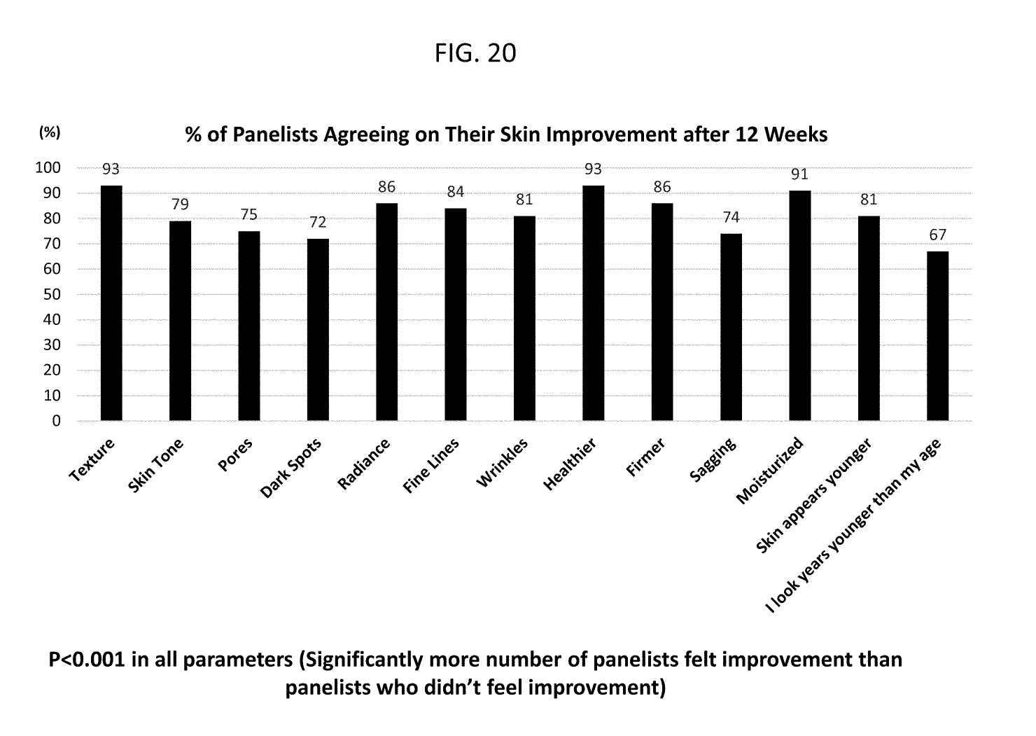

Data on various skin parameters at various times over the 12 week study period are shown in FIGS. 1-19. The results of the subject self-assessment questionnaires are shown in FIG. 20.

As shown in FIG. 1, the number (count), length, width, total size (area) and depth of under eye wrinkles in 56 of the subjects improved after about 12 weeks of treatment. Specifically, about 64% of the subjects showed a reduction in wrinkle count; about 68% of the subjects showed a reduction in wrinkle length, about 61% of the subjects showed a reduction in wrinkle width, about 66% of the subjects showed a reduction in wrinkle area, and about 68% of the subjects showed a reduction in wrinkle depth.

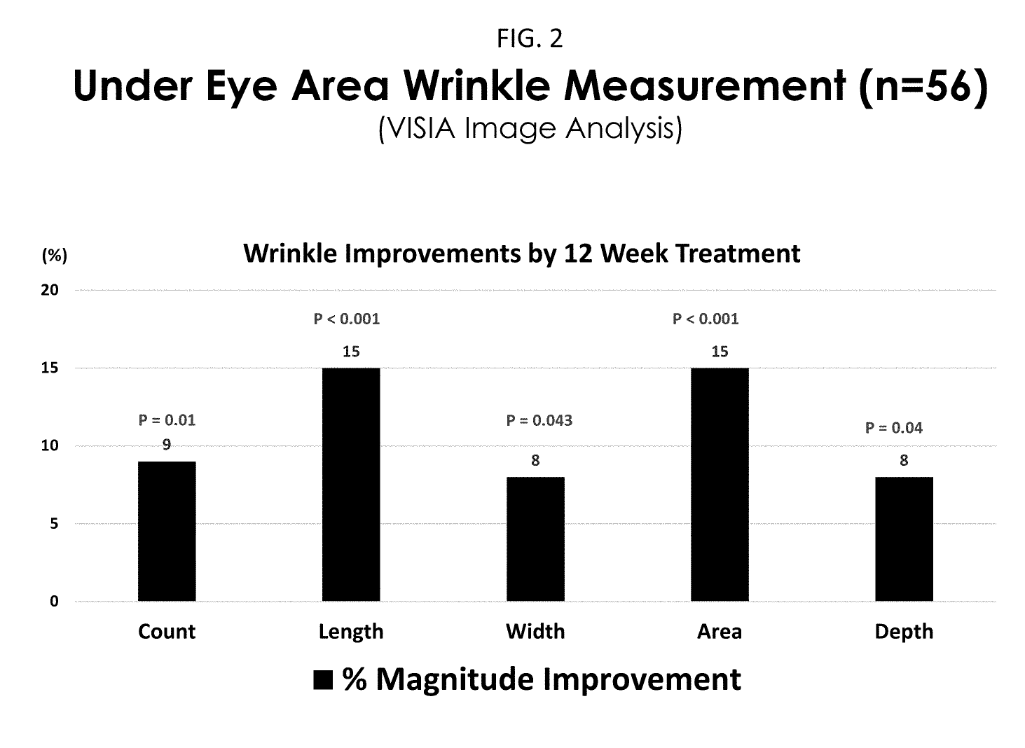

As shown in FIG. 2, the percent magnitude of the improvement in skin health in the 56 subjects of FIG. 1 after about 12 weeks of treatment is shown, in relation to the number (count), length, width, total size (area) and depth of under eye wrinkles in the subjects. Specifically, the magnitude of the reduction in wrinkle count was about 9% (p=0.01); the magnitude of the reduction in wrinkle length was about 15% (p<0.001); the magnitude of the reduction in wrinkle width was about 8% (p=0.043); the magnitude of the reduction in wrinkle area was about 15% (p<0.001); and the magnitude of the reduction in wrinkle depth was about 8% (p=0.04).

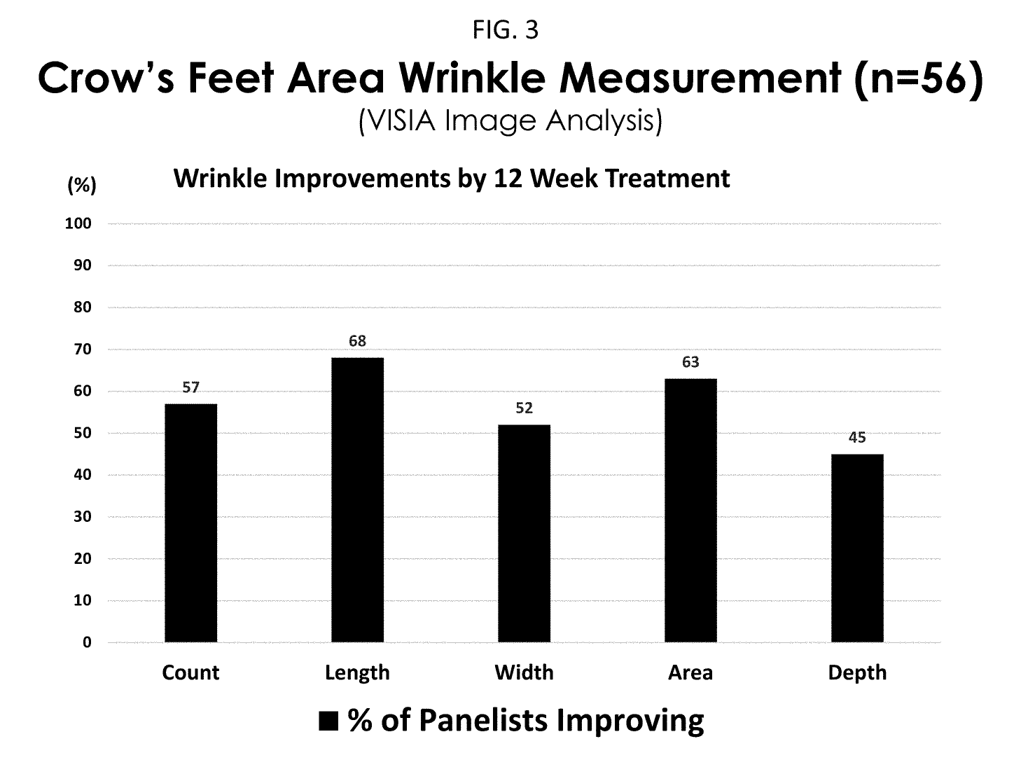

As shown in FIG. 3, the number (count), length, width, total size (area) and depth of wrinkles in the crow's feet area of 56 of the subjects improved after about 12 weeks of treatment. Specifically, about 57% of the subjects showed a reduction in wrinkle count; about 68% of the subjects showed a reduction in wrinkle length, about 52% of the subjects showed a reduction in wrinkle width, about 63% of the subjects showed a reduction in wrinkle area, and about 45% of the subjects showed a reduction in wrinkle depth.

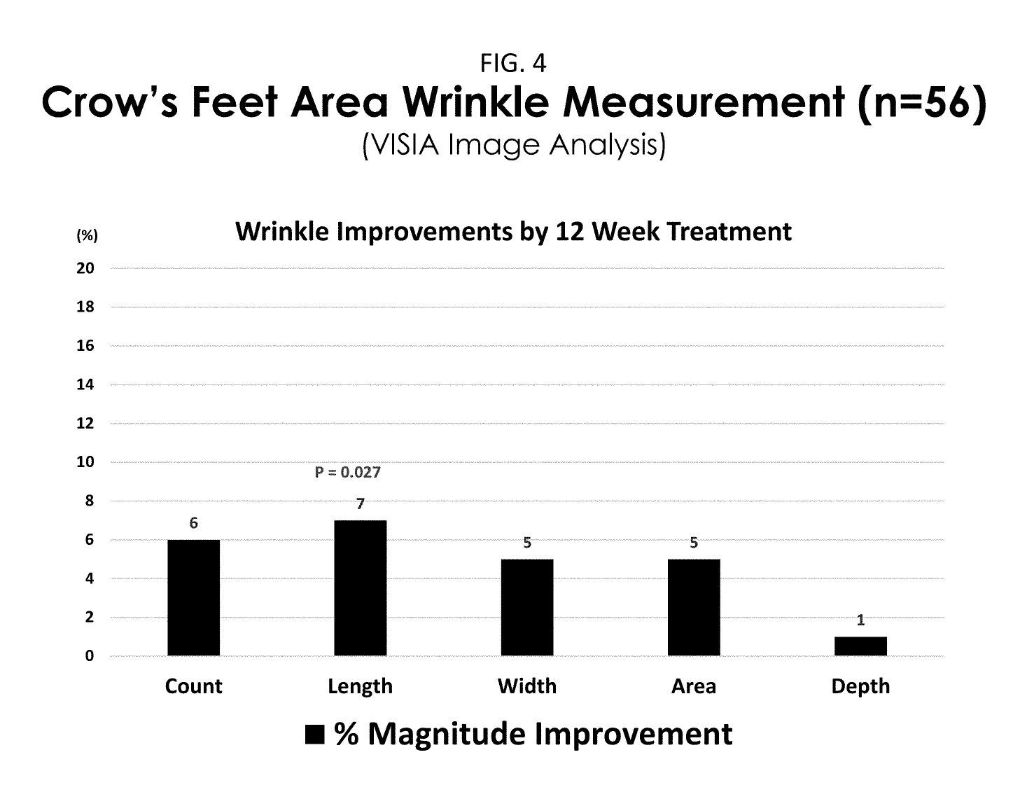

As shown in FIG. 4, the percent magnitude of the improvement in skin health in the 56 subjects of FIG. 3 after about 12 weeks of treatment is shown, in relation to the number (count), length, width, total size (area) and depth of wrinkles in the crow's feet area of the subjects. Specifically, the magnitude of the reduction in wrinkle count was about 6%; the magnitude of the reduction in wrinkle length was about 7% (p=0.027); the magnitude of the reduction in wrinkle width was about 5%; the magnitude of the reduction in wrinkle area was about 5%; and the magnitude of the reduction in wrinkle depth was about 1%.

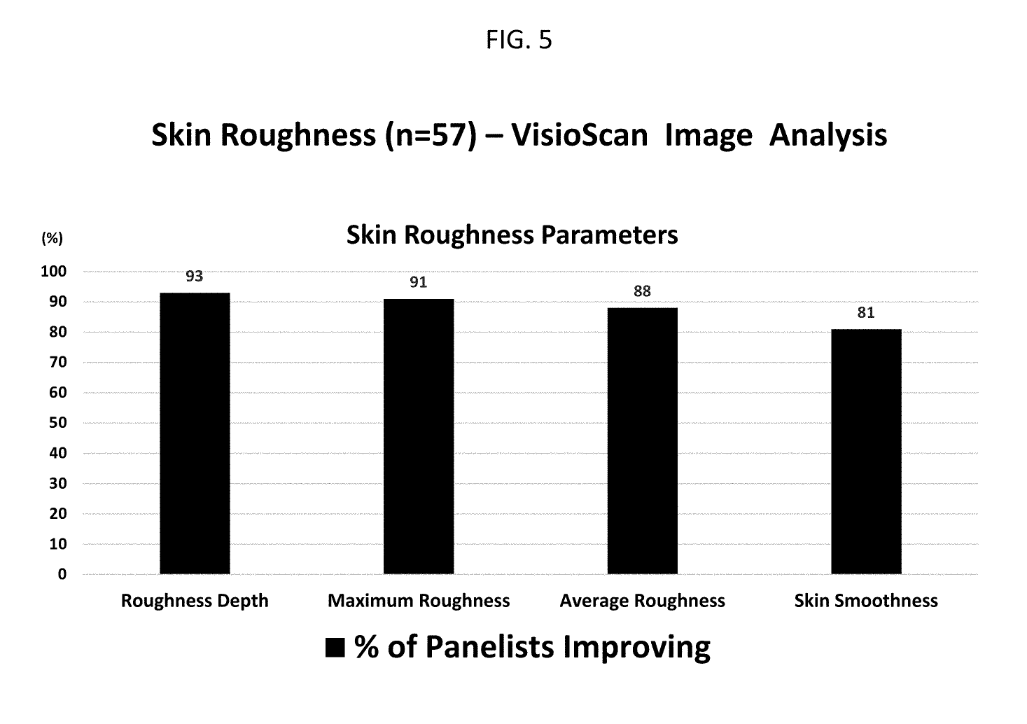

As shown in FIG. 5, the roughness depth, maximum roughness, average roughness and skin smoothness in 57 of the subjects improved after about 12 weeks of treatment. Specifically, about 93% of the subjects showed a reduction in roughness depth; about 91% of the subjects showed a reduction in maximum roughness, about 88% of the subjects showed a reduction in average roughness, and about 81% of the subjects showed an increase in skin smoothness.

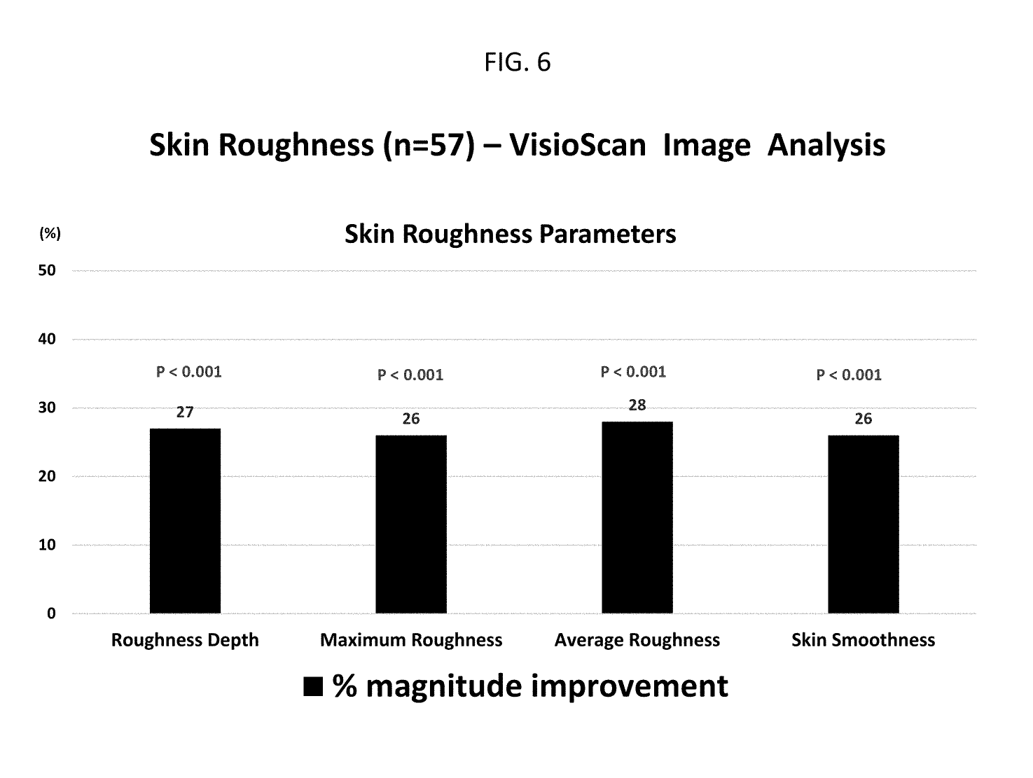

As shown in FIG. 6, the percent magnitude of the improvement in skin health in the 57 subjects of FIG. 5 after about 12 weeks of treatment is shown, in relation to the roughness depth, maximum roughness, average roughness and skin smoothness in the subjects. Specifically, the magnitude of the reduction in roughness depth was about 27% (p<0.001); the magnitude of the reduction in maximum roughness was about 26% (p<0.001); the magnitude of the reduction in average roughness was about 28% ((p<0.001); and the magnitude of the increase in skin smoothness was about 26% (p<0.001).

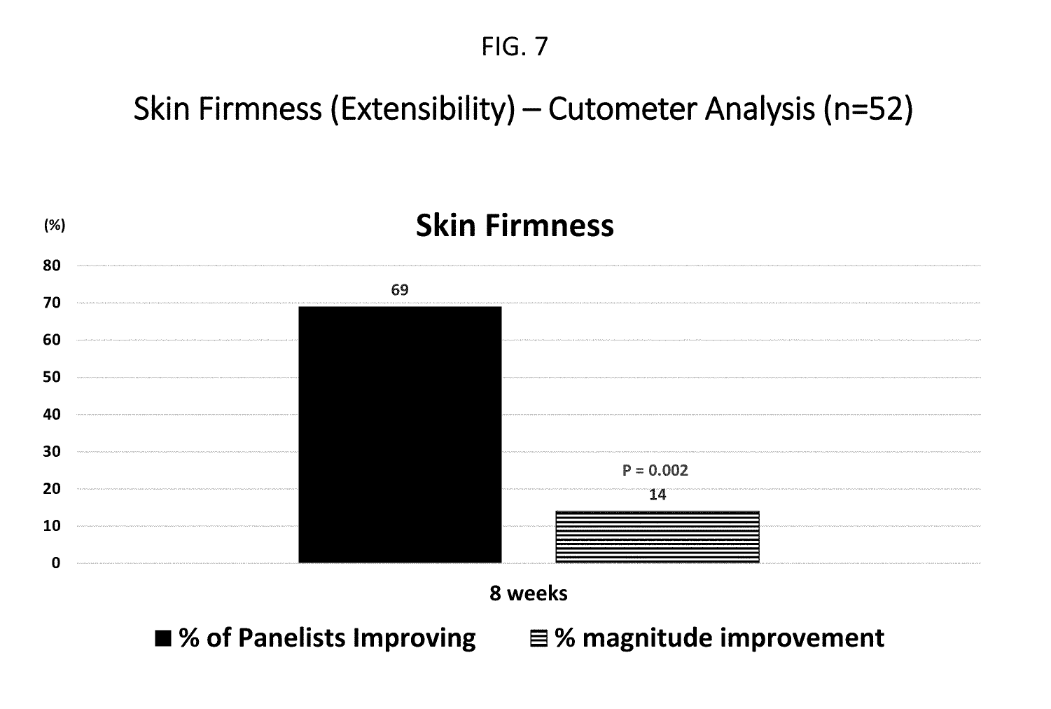

As shown in FIG. 7, about 69% percent of 52 subjects in the study exhibited an improvement in skin firmness after about 8 weeks of treatment as measured with the cutometer, and the magnitude of improvement in skin firmness for these subjects was about 14% (p=0.002).

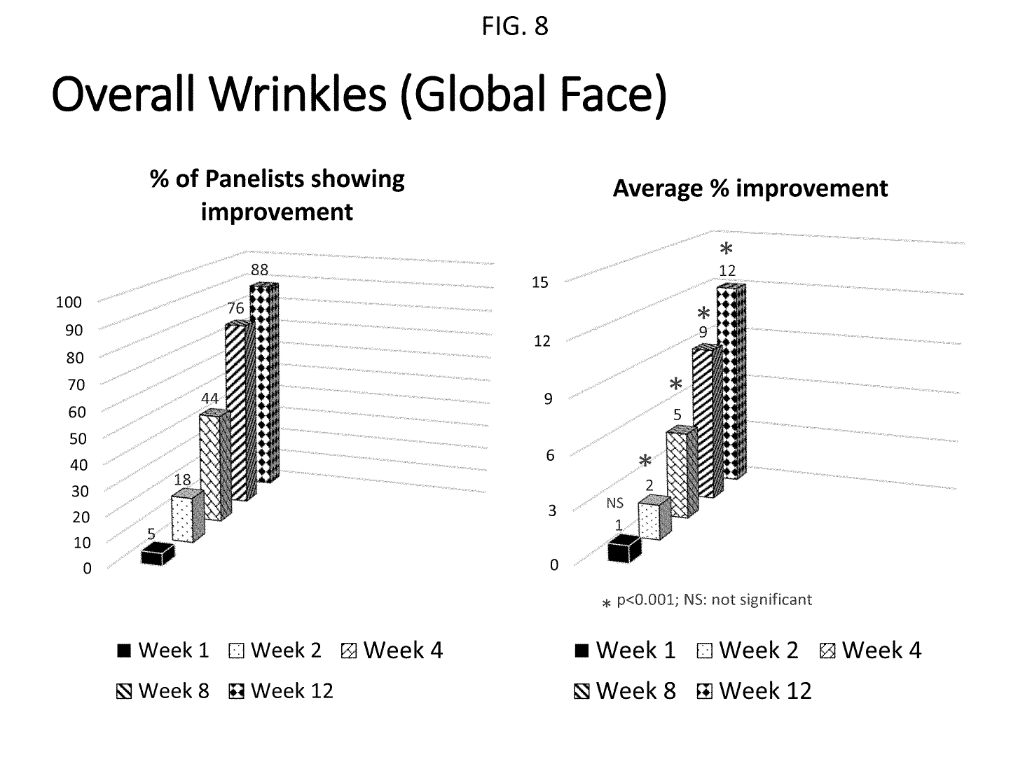

In the left graph of FIG. 8, the percentage of subjects exhibiting an improvement in skin health as measured by an analysis of global facial wrinkles after about 1, about 2, about 4, about 8 and about 12 weeks of treatment is shown in the left graph. The data shows that after about 1 week, about 5% of the subjects showed an improvement in overall facial wrinkles in the global face analysis, about 18% showed an improvement after about 2 weeks, about 44% showed an improvement after about 4 weeks, about 76% showed an improvement after about 8 weeks, and about 88% showed an improvement after about 12 weeks.

The right graph of FIG. 8 shows the average percentage of improvement in the subjects' skin health as measured by an analysis of global facial wrinkles after about 1, about 2, about 4, about 8 and about 12 weeks of treatment. The data shows that after about 1 week, the average percentage of improvement was statistically insignificant; after about 2 weeks, the average percentage of improvement was about 2% (p<0.001); after about 4 weeks, the average percentage of improvement was about 5% (p<0.001); after about 8 weeks, the average percentage of improvement was about 9% (p<0.001); and after about 12 weeks, the average percentage of improvement was about 12% (p<0.001).

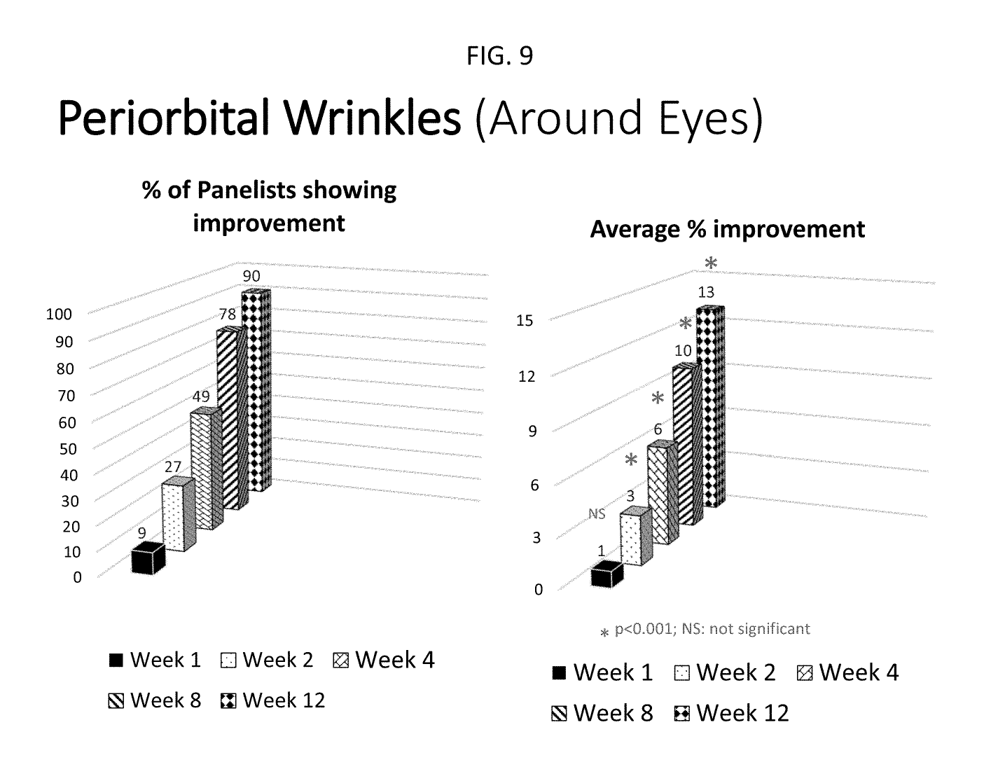

In the left graph of FIG. 9, the percentage of subjects exhibiting an improvement in skin health as measured by an analysis of periorbital wrinkles after about 1, about 2, 4 about, about 8 and about 12 weeks of treatment is shown. The data shows that after about 1 week, about 9% of the subjects showed an improvement in periorbital wrinkles, about 27% showed an improvement after about 2 weeks, about 49% showed an improvement after about 4 weeks, about 78% showed an improvement after about 8 weeks, and about 90% showed an improvement after about 12 weeks.

The right graph of FIG. 9 shows the average percentage of improvement in the subjects' skin health as measured by an analysis of periorbital wrinkles after about 1, about 2, about 4, about 8 and about 12 weeks of treatment. The data shows that after about 1 week, the average percentage of improvement was statistically insignificant; after about 2 weeks, the average percentage of improvement was about 3% (p<0.001); after about 4 weeks, the average percentage of improvement was about 6% (p<0.001); after about 8 weeks, the average percentage of improvement was about 10% (p<0.001); and after about 12 weeks, the average percentage of improvement was about 13% (p<0.001).

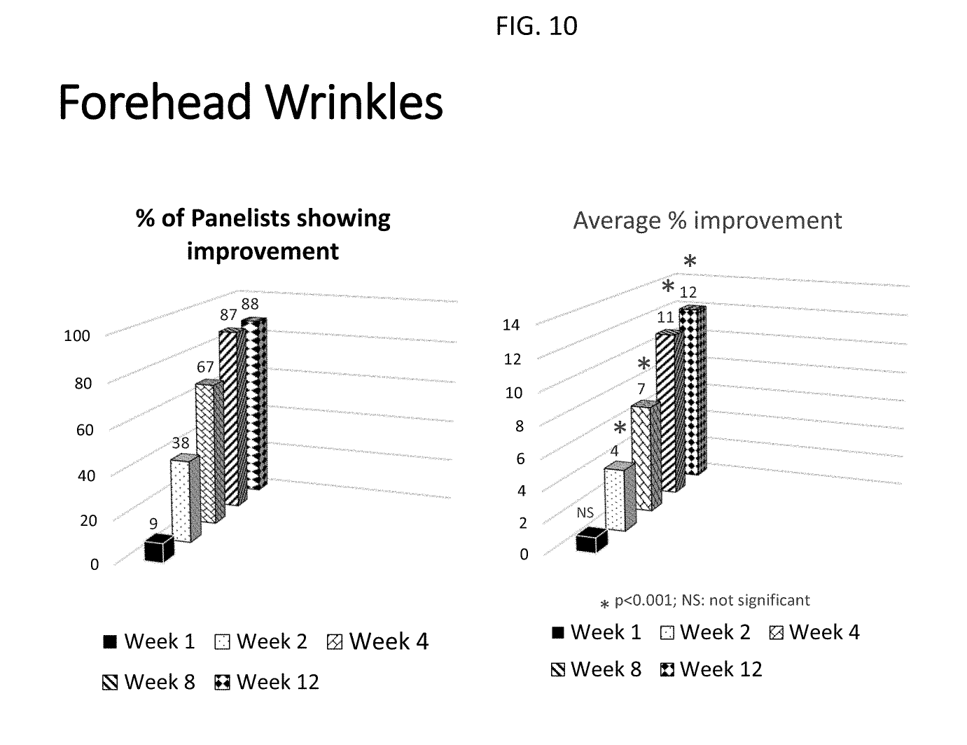

In the left graph of FIG. 10, the percentage of subjects exhibiting an improvement in skin health as measured by an analysis of forehead wrinkles after about 1, about 2, about 4, about 8 and about 12 weeks of treatment is shown. The data shows that after about 1 week, about 9% of the subjects showed an improvement in forehead wrinkles, about 38% showed an improvement after about 2 weeks, about 67% showed an improvement after about 4 weeks, about 87% showed an improvement after about 8 weeks, and about 88% showed an improvement after about 12 weeks.

The right graph of FIG. 10 shows the average percentage of improvement in the subjects' skin health as measured by an analysis of forehead wrinkles after about 1, about 2, about 4, about 8 and about 12 weeks of treatment. The data shows that after about 1 week, the average percentage of improvement was statistically insignificant; after about 2 weeks, the average percentage of improvement was about 4% (p<0.001); after about 4 weeks, the average percentage of improvement was about 7% (p<0.001); after about 8 weeks, the average percentage of improvement was about 11% (p<0.001); and after about 12 weeks, the average percentage of improvement was about 12% (p<0.001).

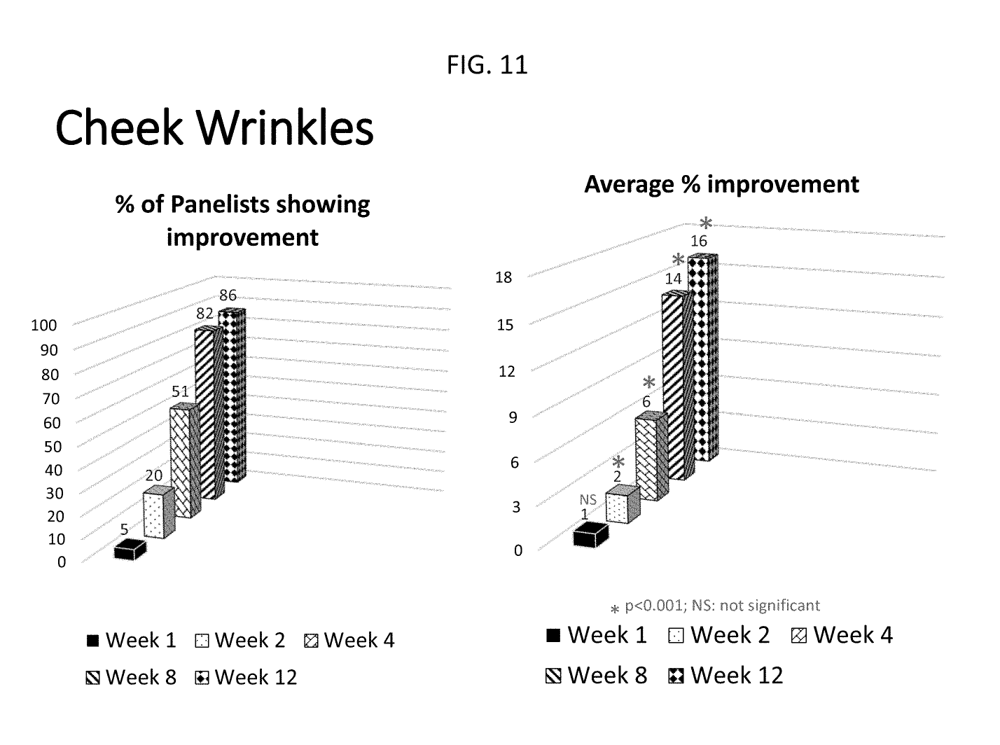

In the left graph of FIG. 11, the percentage of subjects exhibiting an improvement in skin health as measured by an analysis of cheek wrinkles after about 1, about 2, about 4, about 8 and about 12 weeks of treatment is shown. The data shows that after about 1 week, about 5% of the subjects showed an improvement in cheek wrinkles, about 20% showed an improvement after about 2 weeks, about 51% showed an improvement after about 4 weeks, about 82% showed an improvement after about 8 weeks, and about 86% showed an improvement after about 12 weeks.

The right graph of FIG. 11 shows the average percentage of improvement in the subjects' skin health as measured by an analysis of cheek wrinkles after about 1, about 2, about 4, about 8 and about 12 weeks of treatment. The data shows that after about 1 week, the average percentage of improvement was statistically insignificant; after about 2 weeks, the average percentage of improvement was about 2% (p<0.001); after about 4 weeks, the average percentage of improvement was about 6% (p<0.001); after about 8 weeks, the average percentage of improvement was about 14% (p<0.001); and after about 12 weeks, the average percentage of improvement was about 16% (p<0.001).

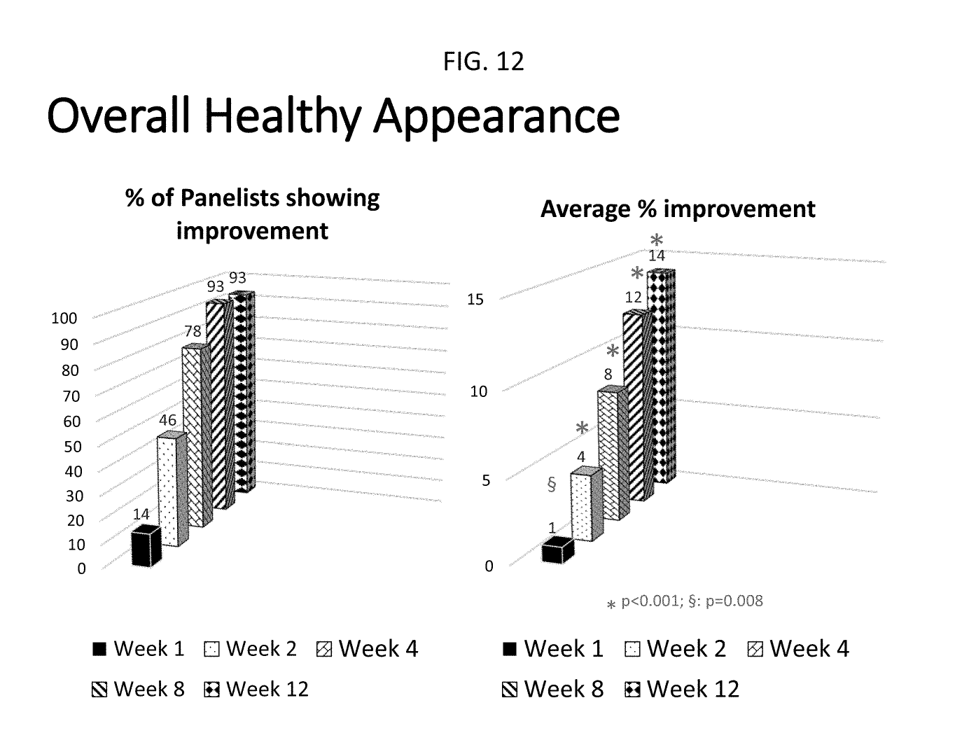

In the left graph of FIG. 12, the percentage of subjects exhibiting an improvement in skin health as measured by an analysis of overall healthy skin appearance after about 1, about 2, about 4, about 8 and about 12 weeks of treatment is shown. The data shows that after about 1 week, about 14% of the subjects showed an improvement in overall healthy skin appearance, about 46% showed an improvement after about 2 weeks, about 78% showed an improvement after about 4 weeks, about 93% showed an improvement after about 8 weeks, and about 93% showed an improvement after about 12 weeks.

The right graph of FIG. 12 shows the average percentage of improvement in the subjects' skin health as measured by an analysis of overall healthy skin appearance after about 1, about 2, about 4, about 8 and about 12 weeks of treatment. The data shows that after about 1 week, the average percentage of improvement was about 1% (p=0.008); after about 2 weeks, the average percentage of improvement was about 4% (p<0.001); after about 4 weeks, the average percentage of improvement was about 8% (p<0.001); after about 8 weeks, the average percentage of improvement was about 12% (p<0.001); and after about 12 weeks, the average percentage of improvement was about 14% (p<0.001).

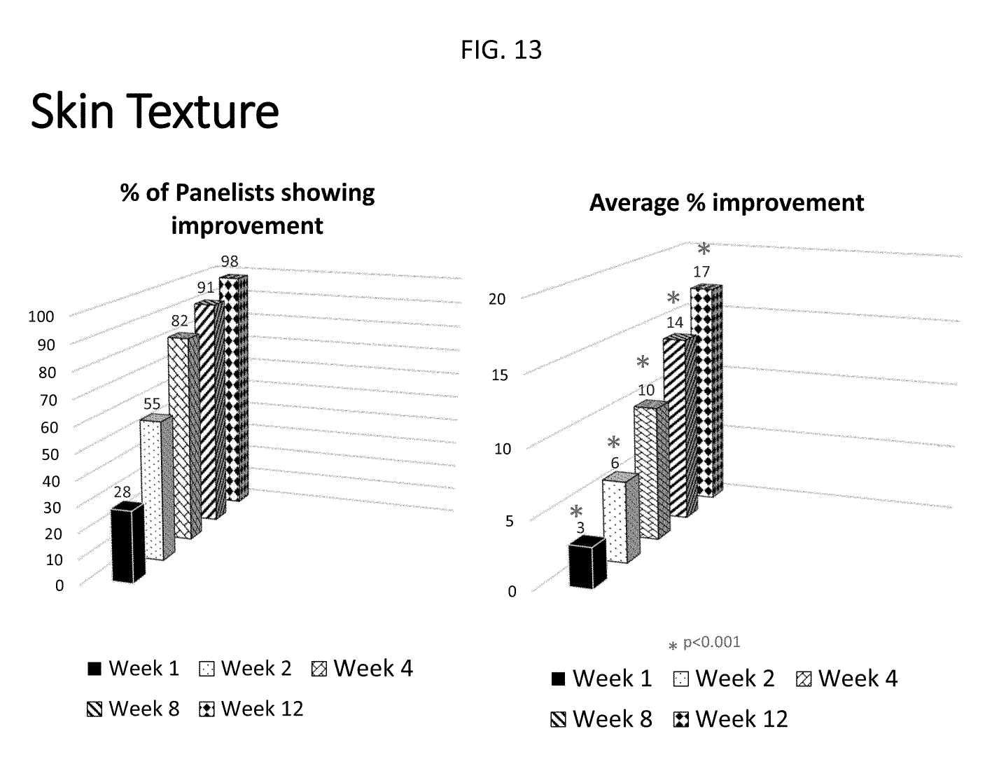

In the left graph of FIG. 13, the percentage of subjects exhibiting an improvement in skin health as measured by an analysis of skin texture after about 1, about 2, about 4, about 8 and about 12 weeks of treatment is shown. The data shows that after about 1 week, about 28% of the subjects showed an improvement in skin texture, about 55% showed an improvement after about 2 weeks, about 82% showed an improvement after about 4 weeks, about 91% showed an improvement after about 8 weeks, and about 98% showed an improvement after about 12 weeks.

The right graph of FIG. 13 shows the average percentage of improvement in the subjects' skin health as measured by an analysis of skin texture after about 1, about 2, about 4, about 8 and about 12 weeks of treatment. The data shows that after about 1 week, the average percentage of improvement was about 3% (p<0.001); after about 2 weeks, the average percentage of improvement was about 6% (p<0.001); after about 4 weeks, the average percentage of improvement was about 10% (p<0.001); after about 8 weeks, the average percentage of improvement was about 14% (p<0.001); and after about 12 weeks, the average percentage of improvement was about 17% (p<0.001).

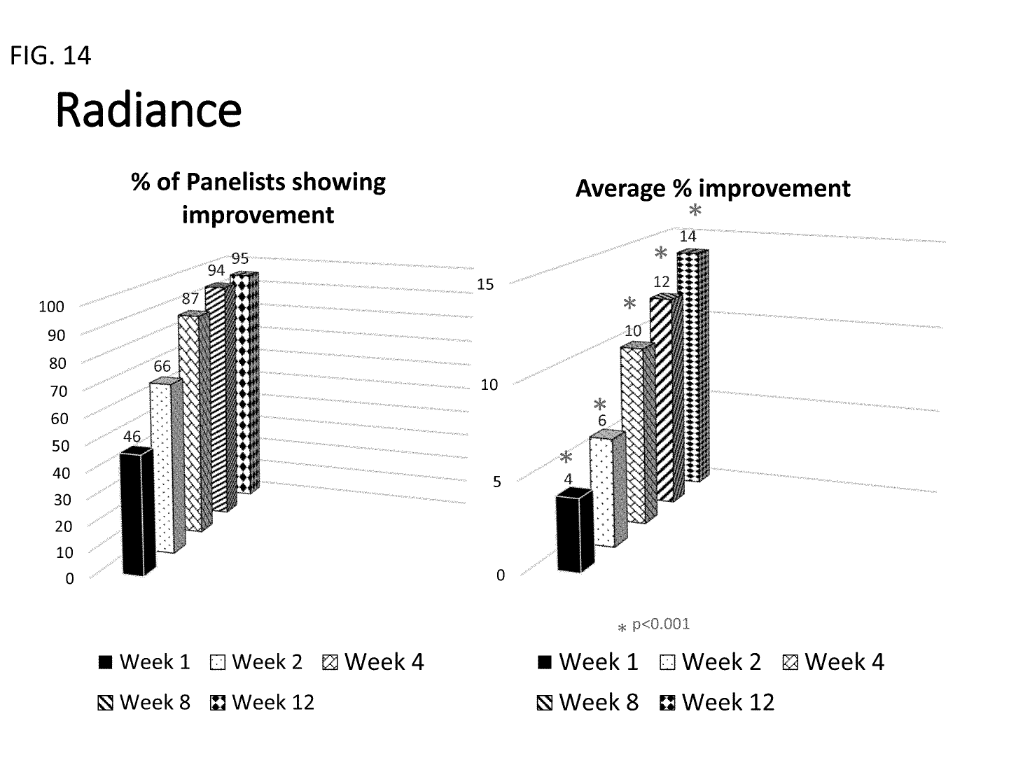

In the left graph of FIG. 14, the percentage of subjects exhibiting an improvement in skin health as measured by an analysis of skin radiance after about 1, about 2, about 4, about 8 and about 12 weeks of treatment is shown. The data shows that after about 1 week, about 46% of the subjects showed an improvement in skin radiance, about 66% showed an improvement after about 2 weeks, about 87% showed an improvement after about 4 weeks, about 94% showed an improvement after about 8 weeks, and about 95% showed an improvement after about 12 weeks.

The right graph of FIG. 14 shows the average percentage of improvement in the subjects' skin health as measured by an analysis of skin radiance after about 1, about 2, about 4, about 8 and about 12 weeks of treatment. The data shows that after about 1 week, the average percentage of improvement was about 4% (p<0.001); after about 2 weeks, the average percentage of improvement was about 6% (p<0.001); after about 4 weeks, the average percentage of improvement was about 10% (p<0.001); after about 8 weeks, the average percentage of improvement was about 12% (p<0.001); and after about 12 weeks, the average percentage of improvement was about 14% (p<0.001).

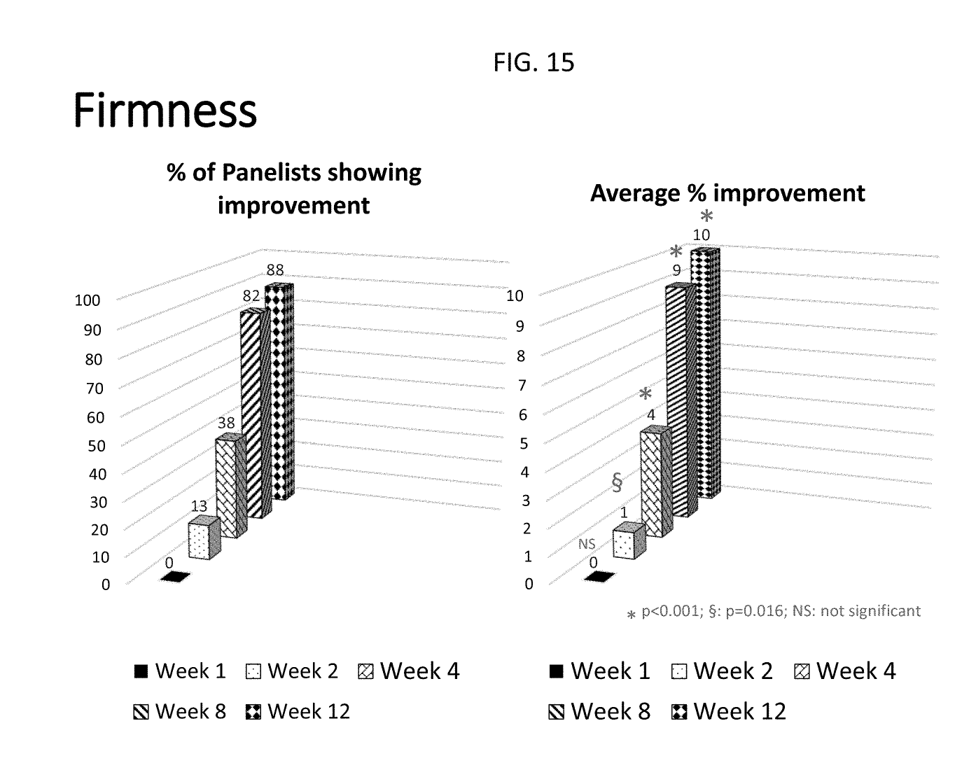

In the left graph of FIG. 15, the percentage of subjects exhibiting an improvement in skin health as measured by an analysis of skin firmness by an expert grader, after about 1, about 2, about 4, about 8 and about 12 weeks of treatment, is shown. The data shows that after about 1 week, none of the subjects showed an improvement in skin firmness, about 13% showed an improvement after about 2 weeks, about 38% showed an improvement after about 4 weeks, about 82% showed an improvement after about 8 weeks, and about 88% showed an improvement after about 12 weeks.

The right graph of FIG. 15 shows the average percentage of improvement in the subjects' skin health as measured by an analysis of skin firmness after about 1, about 2, about 4, about 8 and about 12 weeks of treatment. The data shows that after about 1 week, the average percentage of improvement was statistically insignificant; after about 2 weeks, the average percentage of improvement was about 1% (p=0.016); after about 4 weeks, the average percentage of improvement was about 4% (p<0.001); after about 8 weeks, the average percentage of improvement was about 9% (p<0.001); and after about 12 weeks, the average percentage of improvement was about 10% (p<0.001).

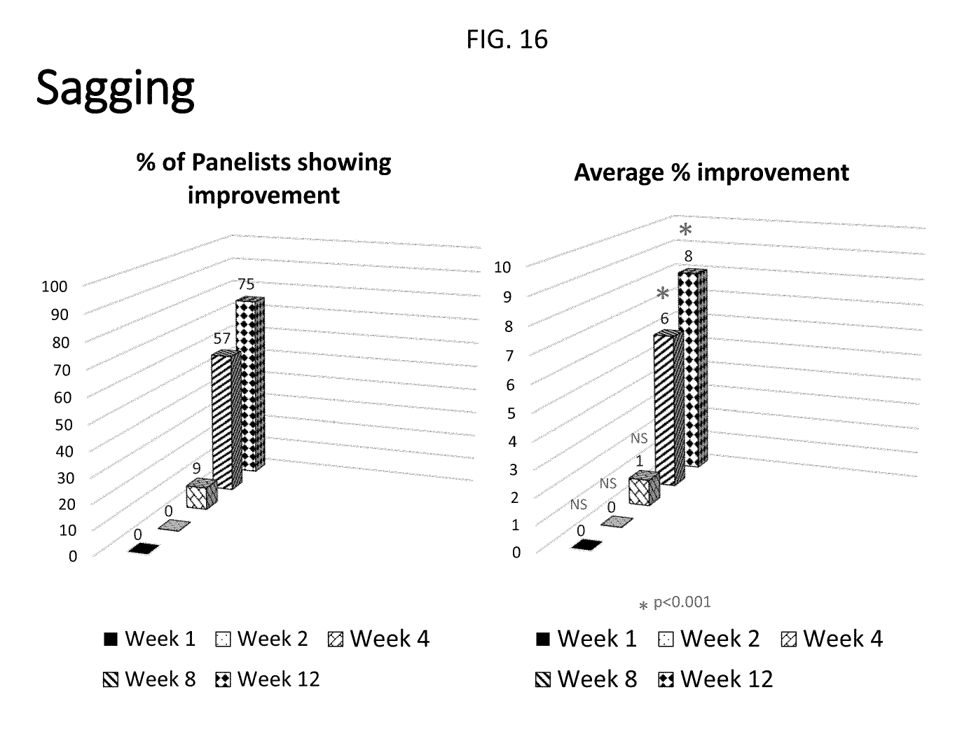

In the left graph of FIG. 16, the percentage of subjects exhibiting an improvement in skin health as measured by an analysis of skin sagging after about 1, about 2, about 4, about 8 and about 12 weeks of treatment is shown. The data shows that after about 1 week and about 2 weeks, none of the subjects showed an improvement in skin sagging, about 9% showed an improvement after about 4 weeks, about 57% showed an improvement after about 8 weeks, and about 75% showed an improvement after about 12 weeks.

The right graph of FIG. 16 shows the average percentage of improvement in the subjects' skin health as measured by an analysis of skin sagging after about 1, about 2, about 4, about 8 and about 12 weeks of treatment. The data shows that after about 1 week and about 2 weeks, the average percentage of improvement was statistically insignificant; after about 4 weeks, the average percentage of improvement was about 1% (statistically insignificant); after about 8 weeks, the average percentage of improvement was about 6% (p<0.001); and after about 12 weeks, the average percentage of improvement was about 8% (p<0.001).

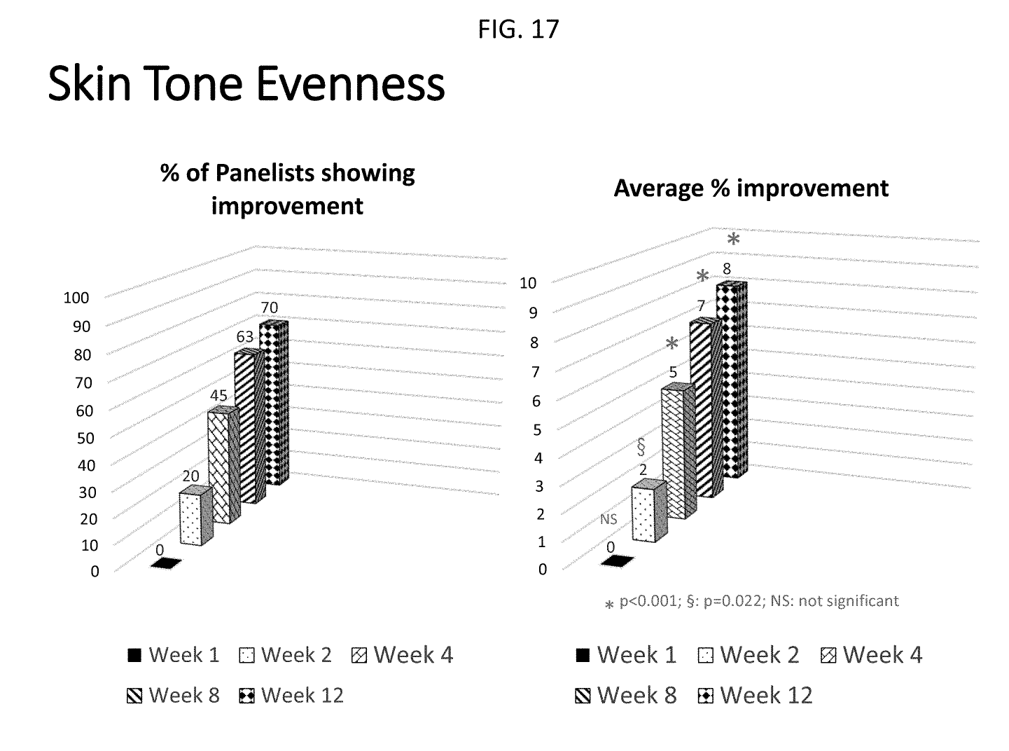

In the left graph of FIG. 17, the percentage of subjects exhibiting an improvement in skin health as measured by an analysis of skin tone evenness after about 1, about 2, about 4, about 8 and about 12 weeks of treatment is shown. This parameter accounts for differences in skin unevenness and discoloration. The data shows that after about 1 week, none of the subjects showed an improvement in skin tone evenness, about 20% showed an improvement after about 2 weeks, about 45% showed an improvement after about 4 weeks, about 63% showed an improvement after about 8 weeks, and about 70% showed an improvement after about 12 weeks.

The right graph of FIG. 17 shows the average percentage of improvement in the subjects' skin health as measured by an analysis of skin tone evenness after about 1, about 2, about 4, about 8 and about 12 weeks of treatment. The data shows that after about 1 week, the average percentage of improvement was statistically insignificant; after about 2 weeks, the average percentage of improvement was about 2% (p=0.022); after about 4 weeks, the average percentage of improvement was about 5% (p<0.001); after about 8 weeks, the average percentage of improvement was about 7% (p<0.001); and after about 12 weeks, the average percentage of improvement was about 8% (p<0.001).

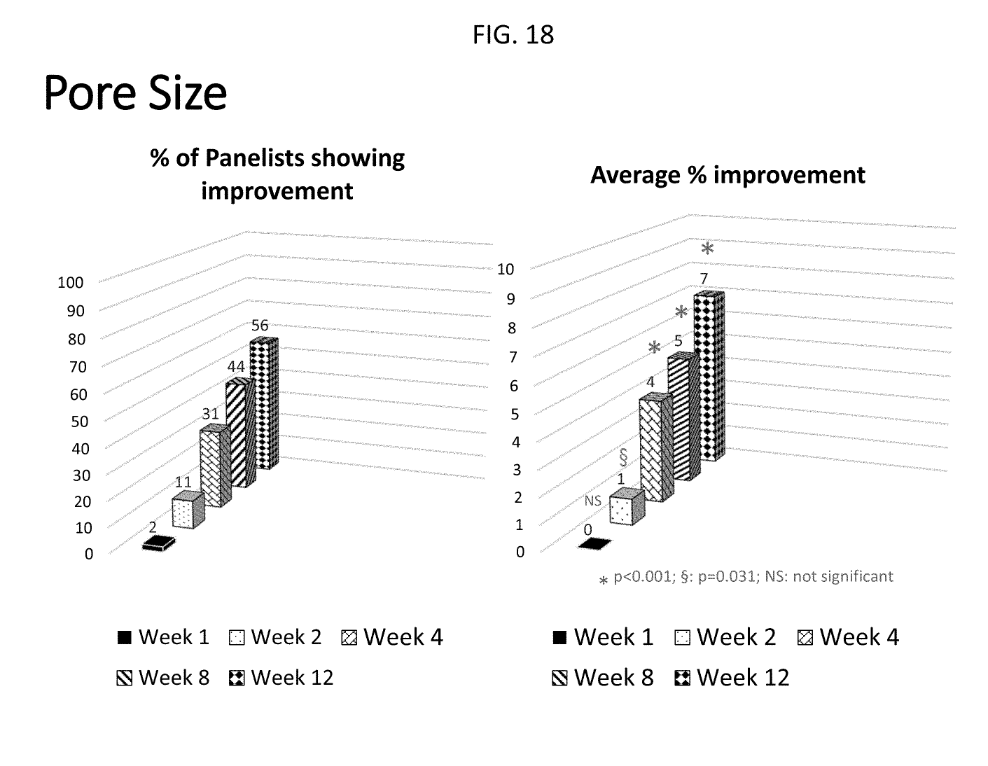

In the left graph of FIG. 18, the percentage of subjects exhibiting an improvement in skin health as measured by an analysis of global facial pore size after about 1, about 2, about 4, about 8 and about 12 weeks of treatment is shown. The data shows that after about 1 week, about 2% of the subjects showed an improvement in pore size, about 11% showed an improvement after about 2 weeks, about 31% showed an improvement after about 4 weeks, about 44% showed an improvement after about 8 weeks, and about 56% showed an improvement after about 12 weeks.

The right graph of FIG. 18 shows the average percentage of improvement in the subjects' skin health as measured by an analysis of global facial pore size after about 1, about 2, about 4, about 8 and about 12 weeks of treatment. The data shows that after about 1 week, the average percentage of improvement was statistically insignificant; after about 2 weeks, the average percentage of improvement was about 1% (p=0.031); after about 4 weeks, the average percentage of improvement was about 4% (p<0.001); after about 8 weeks, the average percentage of improvement was about 5% (p<0.001); and after about 12 weeks, the average percentage of improvement was about 7% (p<0.001).

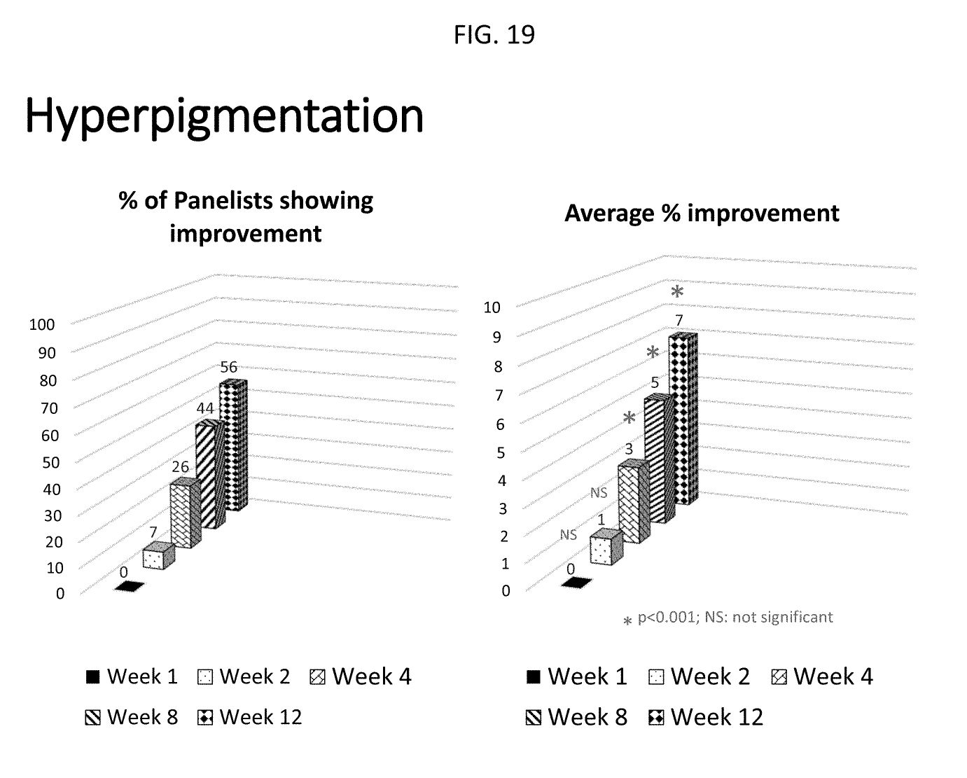

In the left graph of FIG. 19, the percentage of subjects exhibiting an improvement in skin health as measured by an analysis of hyperpigmentation after about 1, about 2, about 4, about 8 and about 12 weeks of treatment is shown. This parameter accounts for differences in dark or mottled skin spots that are strong, intense and/or detectable by eye. The data shows that after about 1 week, none of the subjects showed an improvement in hyperpigmentation, about 7% showed an improvement after about 2 weeks, about 26% showed an improvement after about 4 weeks, about 44% showed an improvement after about 8 weeks, and about 56% showed an improvement after about 12 weeks.

The right graph of FIG. 19 shows the average percentage of improvement in the subjects' skin health as measured by an analysis of hyperpigmentation after about 1, about 2, about 4, about 8 and about 12 weeks of treatment. The data shows that after about 1 week and about 2 weeks, the average percentage of improvement was statistically insignificant; after about 4 weeks, the average percentage of improvement was about 3% (p<0.001); after about 8 weeks, the average percentage of improvement was about 5% (p<0.001); and after about 12 weeks, the average percentage of improvement was about 7% (p<0.001).

The graph shown in FIG. 20 shows the percentage of 57 subjects that perceived an improvement in their skin health in 13 specific parameters after about 12 weeks of following the study treatment (all parameters have a p<0.001). The data shows that a significant number of subjects perceived an improvement in their skin health as evaluated in 13 parameters. Specifically, about 93% of the subjects perceived an improvement in their skin texture, about 79% of the subjects perceived an improvement in their skin tone, about 75% of the subjects perceived an improvement in their pore size, about 72% of the subjects perceived an improvement in their skin hyperpigmentation, about 86% of the subjects perceived an improvement in their skin radiance, about 84% of the subjects perceived an improvement in their fine lines, about 81% of the subjects perceived an improvement in their skin wrinkles, about 93% of the subjects perceived an improvement in their overall skin health, about 86% of the subjects perceived an improvement in their skin firmness, about 74% of the subjects perceived an improvement in their skin sagging, about 91% of the subjects perceived an improvement in their skin moisture levels, about 81% of the subjects perceived an improvement in the appearance of their skin in that their skin appeared younger, and about 67% of the subjects perceived an improvement in their skin in that they looked more than about a year younger than their chronological age.

Thus, the disclosed skin care regimens and methods of improving the appearance of skin by applying to the skin five components (a toner, day moisturizer, night moisturizer, cleanser and serum), each of which include a composition comprising decolorized muscadine pomace solvent extract, beta-glucan, and grape seed extract, are able to significantly improve the appearance of skin as measured by various parameters of skin health, when the concentration of decolorized muscadine pomace solvent extract is about 0.001% to about 1.0% by weight, when the concentration of beta-glucan is about 0.0001% to about 1.0% by weight, and the concentration of grape seed extract is about 0.00001% to about 0.01% by weight. These five components comprise compositions that have low levels of condensed tannins.

### Example 3. DNA Microarray Study Showing the Effectiveness of the Compositions

Metallothionein (MT) gene expression is induced by a high variety of stimuli (e.g., metal exposure or oxidative stress) and is known to bind with wide ranges of metals (e.g., lead, mercury, arsenic, aluminum, cadmium, etc.) both in vivo and in vitro as a means of detoxification of metals to protect cells from damage caused by the exposure to the excessive amount of toxic metals. Metallothionein gene expression is increased in exposure to urban dust. The disclosed skin care methods demonstrating filming/shielding/protecting effect for human skin keratinocyte cells against metals in the air by its binding properties with metals, such as lead and aluminum, and thus it is beneficial for skin health by blocking the heavy metal from direct contacting or permeating skin directly

Topical Muscadine Extract (TME) composition lowered MT gene expressions in human skin keratinocytes during co-treatment with urban dust while MT gene expression was enhanced by exposure to the urban dust 1649b only (without TME). The results suggest that the metals in the urban dust triggered increased MT gene expression, but the TME composition lowered the MT gene expression by blocking the direct contact of metals to the skin keratinocytes. The disclosed skin care methods provide a protecting effect to benefit skin health and to prevent harmful effects caused by the metals in the air. Without being bound by theory, the effect may be lowering the direct contact of metals to the skin, especially when the compositions(s) is/are applied to the skin, such as daily before exposure to the polluted air. There was a reduction of water soluble lead and aluminum species present in urban dust powder 1649b (Signa) after treatment with TME.

DNA microarrays can be used to screen for changes in the expression of different genes. Cultured human keratinocytes were exposed to urban dust alone or urban dust in combination with the test materials. The test materials were TME plus 7 actives (beta glucan, grape seed extract, panthenol, Vitamin A, Vitamin C, Vitamin E, and superoxide dismutase).

After a 24-hour incubation period total RNA was extracted from the cells and then taken through a series of enzyme driven amplification reactions to generate antisense copies of the mRNA. which were suitable for use in array hybridization. The RNA was labeled fluorescently and hybridized overnight with a DNA microarray. After the hybridization reaction, the array was scanned using an array scanner to determine changes in gene expression. The methods are disclosed below:

Human Keratinocyte Cell Culture:

Human epidermal keratinocytes were seeded into culture flasks and grown at 37±2° C. and 5±1% CO₂. Upon reaching confluency the cells were treated with culture media supplemented with either urban dust alone or urban dust in combination with the test material (0.1% Topical Muscadine Extract (TME)+7 other actives in YOUTH Complex [1. Vitamin A as retinyl palmitate with tocopherol: 0.0005%; 2. Vitamin E as tocopheryl acetate: 0.01%; 3. Beta-glucan: 0.005%; 4. Grape Seed Extract: 0.0002%; 5. Vitamin C as magnesium ascorbyl phosphate: 0.0001%; 6. Vitamin B5 (as Panthenol 50%): 0.01%; 7. Superoxide Dismutase: 0.0001%1). A separate flask of cells was treated with culture media alone and acted as an untreated control. The cells were incubated for 24 hours at 37±2° C. and 5±1 CO₂. At the end of the incubation period the culture media was removed via aspiration and the cells were washed once with cold phosphate buffered saline. After removing the wash, the cells were lysed by adding 700 μI of guanidinium thiocyanate lysis solution.

RNA Isolation (Ambion RNAQUEOUS™ Kit):

To the cell lysates prepared above, an equal volume of 64% ethanol was added and the flasks were rocked to mix the contents. After combining, up to 700 μI of the mixture was transferred to a glass fiber filter cartridge, the cartridge was loaded into a 1.5 ml collection tube and the cartridge was centrifuged for 1 minute at 14,000 RPM. The flow through was discarded and any remaining mixture was loaded into the filter cartridge and the centrifugation process was repeated until all of the mixture has been processed. The filter was washed to remove any residual cellular debris from the RNA bound to the glass fibers by subsequently applying 700 μI of wash solution 1 (1 time) and 500 μI of wash solution 2 (2 times) to the filter cartridge and centrifuging at 14,000 RPM for 1 minute to pass each wash through the cartridge. After each wash the flow through was discarded. After the final wash one final spin was performed without wash solution to remove any residual wash solution in the filter cartridge. The RNA bound to the glass fibers within the cartridge was eluted by applying 30 μI of Tris-EDT A buffer (10 mM Tris-HCl, 1 mM EDT A, preheated to 70-80° C.) to the cartridge and centrifuging the cartridge in a new collection tube at 14,000 RPM for one minute. For samples prepared from cell lysates and small tissues the elution process was repeated with an additional 30 μI of preheated TE buffer. For samples prepared from larger tissues (i.e. full thickness tissues) the elution process was repeated two additional times. After the RNA was eluted its concentration was quantified using a RIBOGREEN™ assay.

RNA Concentration Assay (Molecular Probes (RIBOGREEN™ Assay):

The RIBOGREEN™ reagent was provided as a stock solution in DMSO. Prior to use the reagent was diluted 2000-fold in TE buffer. The RNA assay required 200 μI of diluted RIBOGREEN™ reagent per sample to be tested and 1 ml of the reagent for the standards. Once prepared the diluted reagent was stored protected from light. A series of RNA standards was prepared by diluting purified ribosomal RNA derived from E. coli to the following concentrations: 2 μg/ml, 1 μg/ml, 200 ng/ml, 40 ng/ml and O ng/ml (blank). Prior to assaying, the RNA samples prepared above were diluted 1000-fold in TE buffer. For the RNA assay, 100 μI of the diluted samples or standards was transferred to the wells of a 96-well plate. The samples and standards were assayed in duplicate. After the samples/standards were added to the plate. 100 μl of the diluted RIBOGREEN™ assay reagent was added to the wells and the plate was gently mixed and allowed to incubate for 5-10 minutes protected from the light. After this incubation, the plate was read with a fluorimeter using an excitation wavelength of 480 nm and an emission wavelength of 525 nm.

mRNA Amplification (Ambion MESSAGEAMP™ aRNA Kit):

First Strand cDNA Synthesis: To start the first strand synthesis, 5 μg of total RNA for each sample was added to 600 μI PCR tubes and the total volume of liquid in the tube was adjusted to 12 μI with DEPC H₂O. To each tube, 1 μI of T7 Oligo(dT) primer was added and the tube was incubated at 70±2° C. for 10 minutes to denature the RNA and then placed on ice to allow the primer to anneal to the poly A ends of the mRNA. After cooling 2 μI of 10×first strand buffer, 1 μI of RNAse inhibitor and 4 μI of dNTP Mix was added to each tube, and the tube was placed at 42° C. As soon as the tube is heated, 1 μI of reverse transcriptase was added and the tubes were returned to 42±2° C. for 2 hours. At the end of the two hours the tubes were briefly centrifuged to collect all of the fluid at the bottom of the tube and then placed on ice.

Second Strand Synthesis and cDNA Purification:

For the synthesis of the second strand of cDNA the following items were added to the tubes above (in this order): 63 μI DEPC H₂O, 1 O μ110×second strand buffer, 4 μI dNTP mix, 2 μI DNA Polymerase and 1 μI of RNAse H. The tube was mixed and then incubated at 16±2° C. for 2 hours. Towards the end of the 2 hour incubation a sufficient quantity of DEPC H₂O was warmed to 50±2° C. and a cDNA purification filter cartridge was equilibrated with 50 μI of cDNA binding buffer (one cartridge per sample) for at least 5 minutes. After the samples finished incubating, 250 μI of cDNA binding buffer was added to each tube and thoroughly mixed. The contents of the PCR tube was transferred to the cDNA purification filter cartridge. The cartridge was placed in a collection tube and centrifuged at 10,000 RPM for 1 minute. The flow-through was discarded and 650 μI of cDNA wash solution was added to the cartridge. The cartridge was centrifuged again and the flow through was discarded, and then centrifuged one last time to ensure that the wash buffer had been completely emptied from the filter. The cDNA was eluted by applying 10 μI of preheated DEPC H₂O to the filter and centrifuging the filter in a new collection tube at 10,000 RPM for one minute. This elution was performed one additional time to give a total volume of 16-18 μl of cDNA solution.

In Vitro Transcription to Synthesize aRNA and aRNA Purification:

The in vitro transcription was begun by adding the following to the cDNA solution: 3 μI of 50 mM aaUTP, 12 μI of ATP/CTP/GTP Mix (25 mM), 3 μI of 50 mM UTP, 4 μl of 10×Reaction buffer, and 4 μI of T7 enzyme mix. The tube was mixed and then incubated at 37±2° C. for 6-14 hours. Towards the end of the incubation a sufficient volume of Elution Solution was warmed to 50-60° C. and an aRNA filter cartridge was equilibrated with 100 μI of aRNA binding buffer for at least 5 minutes. At the end of the incubation period, 350 μI of aRNA binding buffer was added to the sample tubes and thoroughly mixed. An additional 250 μI of absolute ethanol was also added to each tube. The mixture was then transferred to an aRNA filter cartridge; the cartridge was inserted into a collection tube and centrifuged at 10,000 RPM for 1 minute. The flow-through was discarded and 650 μI of aRNA wash buffer was added to the cartridge followed by centrifuging at 10,000 RPM for one minute. After discarding the flow through the cartridge was spun one final time to remove all traces of the wash buffer. The cartridge was then transferred to a new collection tube 25 μI of prewarmed Elution Solution was added to the cartridge. The cartridge was incubated for 2 minutes at room temperature and then aRNA was eluted by centrifuging for 1 minute at 10,000 RPM. This elution was performed one additional time to give a total volume of 45-50 μI of aRNA solution. The final concentration of the aRNA was determined by the RIBOGREEN™ assay described above.

aRNA: Dye Coupling Reaction:

For the dye coupling reaction, 2 μg of aRNA was vacuum dried and suspended in 4.5 μI of coupling buffer. 5.5 μI of dye mix (Cy3 or Cy5) was then added and the mixture was incubated for 30 minutes at room temperature in the dark. After this incubation, 2.5 μI of 4M hydroxylamine was added and the mixture was incubated for an additional 15 minutes at room temperature in the dark.

Labeled aRNA Purification:

To purify the labeled aRNA, a microcon YM-30 filter column was inserted into a collection tube and filled with 400 μI of TE buffer. The Cy3 and Cy5 probes was combined (12.5 μI of each) and then added to the microcon filter and thoroughly mixed with the TE buffer. The filter was centrifuged at 12,000 RPM for 8 minutes and the flow through was discarded. The column was then washed twice with 400 μI of TE buffer, discarding the flow through each time. After the final wash the filter column was inverted, placed into a new collection tube and centrifuged at 12,000 RPM for 2 minutes to collect the probe.

Microarray Hybridization and Washing (Agilent Technologies Microarrays):

For hybridization, 11 μI of 1 Ox control target RNA (supplied with Agilent Technologies In Situ Hybridization Kit) was mixed with 30 μJ of DEPC water and 2.2 μl of 25× Agilent Fragmentation Buffer. This mixture was incubated at 65° C. for approximately 30 minutes in a hybridization oven. At the end of the incubation 55 μI of Agilent Hybridization Buffer was added along with the fluorescent aRNA probes prepared above. An Agilent SUREHYB® hybridization chamber was prepared by inserting a glass gasket slide into the bottom half of the chamber. At the end of the incubation, the hybridization mixture (approximately 110 μl) was applied to the glass gasket slide and an Agilent Microarray Chip was placed face down on top of this gasket such that the hybridization solution is sandwiched between the glass gasket slide and the microarray face of the chip. The top half of the chamber was then attached and the connecting thumbscrew tightened. After verifying that there was good bubble formation in the chamber, it was placed into the hybridization oven for approximately 17 hours (65° C. and rotating at 4 RPM). At the end of the hybridization period, the microarray/glass gasket was removed from the SUREHYB® chamber and placed in 50 ml of wash solution 1 (room temperature, 6×SSC, 0.005% Triton X-102). After the gasket had fallen away from the microarray, the array was transferred to 300 ml of fresh wash solution 1 on a magnetic stir plate. The array was washed while the solution is mixed at medium speed for 10 minutes and then transferred to 300 ml of wash solution 2 (0.1×SSX, 0.005% Triton X-102, 4° C.) for 5 minutes. After the final wash, the array was centrifuged at 500 RPM for 5 minutes to dry it.

Microarray Scanning and Analysis:

The microarrays were scanned with an Axon GENEPIX® 41 00A Scanner with the scanning resolution set to 10 μm and analyzed with GENEPIX® Pro software. During the initial scan, the PMT gains for the scanner was adjusted such that the cy5/cy3 image count ratios are between 0.9 and 1.1.

### Calculations

RNA RIBOGREEN™ Assay:

To derive the standard curve for the RIBOGREEN™ assay, the relative fluorescent units versus the known RNA concentrations in μg/ml for the standards was plotted and subjected to regression analysis to establish the Une that best fits these data points. Mean RFU values for the test materials and untreated samples were used to estimate the amount of RNA present in each sample.

Microarray Calculations:

The level of gene expression is related to the fluorescence intensity of the probed gene marker on the microarray. Because it is possible to have differences in labeling efficiency when making the Cy3 and Cy5 probes the fluorescence measurements between the two respective dyes were normalized before evaluating changes in gene expression. Fluorescence intensities for the microarrays were subjected to global normalization. The total fluorescent signal for both dyes was normalized with a correction factor that would make the ratio of total intensities for both dyes equal to one.

Keratinocytes were treated with a disclosed composition and urban dust, or were not treated and just exposed to urban dust. The results are shown below:

| | | Gene | |
|---|---|---|---|
| | Gene | Expression | |
| | Expression | Ratio | |
| | Ratio | (TME&7 + | |
| | (Urban | UD | |
| Gene | Dust vs. | vs. | |
| Name | Untreated | Untreated) | Gene Description |
| MT1A | 2.365 | 0.425 | Homo sapiens metallothionein 1A (MT1A), mRNA |
| | | | [NM_005946] |
| MT1B | 2.672 | 0.701 | Homo sapiens metallothionein 1B (MT1B), mRNA |
| | | | [NM_005947] |
| MT1E | 2.578 | 1.062 | Homo sapiens metallothionein 1E (MT1E), mRNA |
| | | | [NM_175617] |
| MT1F | 1.739 | 1.038 | Homo sapiens metallothionein 1F (MT1F), mRNA |
| | | | [NM_005949] |
| MT1G | 2.56 | 0.582 | Homo sapiens metallothionein1G (MT1G), mRNA |
| | | | [NM_005950] |
| MT1G | 2.421 | 0.631 | Homo sapiens metallothionein 1G (MT1G), mRNA |
| | | | [NM_005950] |
| MT1H | 2.194 | 0.408 | Homo sapiens metallothionein 1H (MT1H), mRNA |
| | | | [NM_005951] |
| MT1H | 1.998 | 0.503 | Homo sapiens metallothionein 1H (MT1H), mRNA |
| | | | [NM_005951] |
| MT1M | 2.393 | 1.62 | Homo sapiens metallothionein 1M (MT1M), mRNA |
| | | | [NM_176870] |
| MT1X | 2.513 | 0.492 | Homo sapiens metallothionein 1X (MT1X), mRNA |
| | | | [NM_005952] |
| MT1X | 2.021 | 0.562 | Homo sapiens metallothionein 1X (MT1X), mRNA |
| | | | [NM_005952] |
| MT2A | 5.697 | 4.308 | Homo sapiens metallothionein 2A (MT2A), mRNA |
| | | | [NM_005953] |
| MT2A | 5.987 | 3.67 | Metallothionein-2 (MT-2) (Metallothionein-II) (MT-II) |
| | | | (Metallothionein-2A). [Source: Uniprot/SWISSPROT; |
| | | | Acc: P02795] [ENST00000245185] |
| MT3 | 1.307 | 0.708 | Homo sapiens metallothionein 3 (MT3), mRNA |
| | | | [NM_005954] |

All GENBANK ® Accession numbers incorporated by reference as available on Apr. 30, 2018.

These results show that metallothionein gene expression was reduced when TME with the additional actives (see above) were used. This evidences that the disclosed compositions protect skin cells from metal exposure, such as from urban dust which contains heavy metals. The results show a protective effect of the composition for skin cells.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A combination comprising five components in separate containers, wherein the five components are a toner composition, a day moisturizer composition, a night moisturizer composition, a cleanser composition and a serum composition,
wherein each of the components comprise:
(i) decolorized muscadine (Vitis rotundifolia) pomace solvent extract comprising a liquid bronze muscadine pomace extract combined with a liquid purple muscadine pomace extract to produce a liquid muscadine pomace extract, wherein a) the bronze muscadine pomace extract and the purple muscadine pomace extract are aqueous extracts; b) the mixture of bronze muscadine pomace extract and purple muscadine pomace extract promotes solubility of ellagic acid in the muscadine pomace extract; c) the bronze muscadine pomace extract and the purple muscadine pomace extract are filtered and fermented extracts; and d) the muscadine pomace extract has a polyphenol content of at least about 2%;
(ii) beta-glucan, and
(iii) grape seed extract.

2. The combination of claim 1, wherein the toner composition comprises
about 0.001% to about 1.0% of decolorized muscadine pomace solvent extract by weight of the toner composition,
about 0.000001% to about 0.1% beta-glucan by weight of the toner composition, and
about 0.00001% to about 0.01% grape seed extract by weight of the toner composition.

3. The combination of claim 1, wherein the day moisturizer composition comprises
about 0.001% to about 1.0% of decolorized muscadine pomace solvent extract by weight of the day moisturizer composition,
about 0.0001% to about 0.1% beta-glucan by weight of the day moisturizer composition, and
about 0.00001% to about 0.01% grape seed extract by weight of the day moisturizer composition.

4. The combination of claim 1, wherein the night moisturizer composition comprises
about 0.001% to about 1.0% of decolorized muscadine pomace solvent extract by weight of the night moisturizer composition,
about 0.000001% to about 0.1% beta-glucan by weight of the night moisturizer composition, and
about 0.00001% to about 0.01% grape seed extract by weight of the night moisturizer composition.

5. The combination of claim 1, wherein the cleanser composition comprises
about 0.001% to about 1.0% of decolorized muscadine pomace solvent extract by weight of the cleanser composition,
about 0.000001% to about 0.1% beta-glucan by weight of the cleanser composition, and
about 0.00001% to about 0.01% grape seed extract by weight of the cleanser composition.

6. The combination of claim 1, wherein the serum composition comprises
about 0.01% to about 10.0% of decolorized muscadine pomace solvent extract by weight of the serum composition,
about 0.00001% to about 1.0% beta-glucan by weight of the serum composition, and
about 0.0001% to about 0.1% grape seed extract by weight of the serum composition.

7. The combination of claim 2, wherein the toner composition comprises
about 0.01% to about 0.05% of decolorized muscadine pomace solvent extract by weight of the toner composition,
about 0.000001% to about 0.01% beta-glucan by weight of the toner composition, and
about 0.0001% to about 0.001% grape seed extract by weight of the toner composition.

8. The combination of claim 3, wherein the day moisturizer composition comprises
about 0.01% to about 0.05% of decolorized muscadine pomace solvent extract by weight of the day moisturizer composition,
about 0.001% to about 0.01% beta-glucan by weight of the day moisturizer composition, and
about 0.0001% to about 0.001% grape seed extract by weight of the day moisturizer composition.

9. The combination of claim 4, wherein the night moisturizer composition comprises
about 0.01% to about 0.05% of decolorized muscadine pomace solvent extract by weight of the night moisturizer composition,
about 0.000001% to about 0.01% beta-glucan by weight of the night moisturizer composition, and
about 0.0001% to about 0.001% grape seed extract by weight of the night moisturizer composition.

10. The combination of claim 5, wherein the cleanser composition comprises
about 0.01% to about 0.05% of decolorized muscadine pomace solvent extract by weight of the cleanser composition,
about 0.000001% to about 0.01% beta-glucan by weight of the cleanser composition, and
about 0.0001% to about 0.001% grape seed extract by weight of the cleanser composition.

11. The combination of claim 6, wherein the serum composition comprises
about 0.1% to about 0.5% of decolorized muscadine pomace solvent extract by weight of the serum composition,
about 0.00001% to about 0.1% beta-glucan by weight of the serum composition, and
about 0.005% to about 0.1% grape seed extract by weight of the serum composition.

12. The combination of claim 1, wherein at least one of the five components further comprises a sunscreen.

13. The combination of claim 1, wherein at least one of the five components further comprises panthenol, Vitamin A, Vitamin C, Vitamin E, superoxide dismutase, or any combination thereof.

14. The combination of claim 13, wherein at least one of the five components comprises by weight of the component,
about 0.0001% to about 1.0% panthenol,
about 0.00005% to about 1.0% Vitamin A,
about 0.00001 to about 0.1% Vitamin C,
about 0.001% to about 1.0% Vitamin E,
about 0.00000001% to about 0.1% superoxide dismutase,
or any combination thereof.

15. The combination of claim 13, wherein the at least one of the five components comprises magnesium ascorbyl phosphate.

16. The combination of claim 13, at least one of the five components comprises Vitamin E acetate.

17. The combination of claim 13, wherein at least one of the five components comprises Vitamin A palmitate or retinol.

18. The combination of claim 1, wherein at least one of the five components further comprises lotus japonicus extract, schizandra chinensis fruit extract, or both.

19. The combination of claim 1, wherein the ratio of bronze muscadine pomace extract to purple muscadine pomace extract of the decolorized muscadine pomace solvent extract in each of the five components ranges from about 0.1 to about 10 (weight to weight).

20. The combination of claim 19, wherein the ratio of bronze muscadine pomace extract to purple muscadine pomace extract of the decolorized muscadine pomace solvent extract in each of the five components ranges from about 0.3 to about 3 (weight to weight).

21. The combination of claim 1, wherein the decolorized muscadine pomace solvent extract in each of the five components comprises about 7% to about 10% polyphenols and less than about 5% monosaccharides by weight of the decolorized muscadine pomace solvent extract, and wherein the condensed tannins are less than about 10% of the total polyphenol content of the decolorized muscadine pomace solvent extract.

22. The combination of claim 1, wherein the total polyphenols of the decolorized muscadine pomace solvent extract in each of the five components consist of at least about 85% polyphenols other than condensed tannins.

23. The combination of claim 1, wherein the decolorized muscadine pomace solvent extract in each of the five components further comprises about 0.5% to about 5% fiber, about 7% to about 14% protein, about 0.05% to about 3% fat and about 15 to about 20% organic acids by weight of the decolorized muscadine pomace solvent extract.

24. The combination of claim 23, wherein the decolorized muscadine pomace solvent extract in each of the five components further comprises about 1% to about 2% fiber, about 7% to about 8% protein, about 0.5% to about 1.5% fat and about 15.5% to about 16.5% organic acids by weight of the decolorized muscadine pomace solvent extract.

25. The combination of claim 1, wherein the phenolic content of the decolorized muscadine pomace solvent extract in each of the five components comprises about 2 to about 3% ellagic acid and about 30 to about 31% gallic acid by weight of the decolorized muscadine pomace solvent extract.

26. The combination of claim 25, wherein phenolic content of the decolorized muscadine pomace solvent extract in each of the five components comprises about 2 to about 3% ellagic acid, about 3 to about 4% ellagic acid glycosides, about 30 to about 31% gallic acid, about 2 to about 3% quercetin, about 10 to about 11% gallotannins, about 7 to about 8% ellagitannins, about 29 to about 30% proanthocyanidins, about 4 to about 5% anthocyanins, about 2 to about 3% catechins, and about 6 to about 7% phenolic acids by weight of the decolorized muscadine pomace solvent extract.

27. The combination of claim 1, wherein the purple muscadine pomace extract in at least one of the five components comprises an extract of whole purple muscadine grapes, an extract of purple muscadine pomace from other than whole grapes, or both.

28. A skin care method, comprising:
applying to the skin an effective amount of the combination of claim 1,
wherein each of the five components of the combination are applied for a period of time sufficient to improve the appearance of skin.

29. The skin care method of claim 28, wherein the skin surface is a facial skin surface.

30. The skin care method of claim 28, wherein improving the appearance of the skin comprises reducing at least one of: the depth of wrinkles in the skin; the number of wrinkles in the skin; the length of wrinkles in the skin; or the width of wrinkles in the skin.

31. The skin care method of claim 30, wherein the wrinkles are periorbital wrinkles, forehead wrinkles, or cheek wrinkles.

32. The skin care method of claim 28, wherein improving the appearance of the skin comprises at least one of: reducing skin roughness, increasing skin smoothness, increasing skin radiance, increasing skin firmness, reducing skin sagging, increasing the evenness of skin tone, reducing pore size, or reducing skin hyperpigmentation.

33. The skin care method of claim 28, wherein the five combination is applied to the skin:
a) at least once a day for at least about one week;
b) at least twice a day for at least about one week;
c) at least once a day for at least about twelve weeks; or
d) at least twice a day for at least about twelve weeks.

34. The skin care method of claim 28, comprising:
a) applying an effective amount of first application of the cleanser composition the skin in the morning;
b) rinsing the first application cleaner composition from the skin;
c) applying an effective amount of the day moisturizer composition and an effective amount of the serum composition following step b):
d) applying an effective amount of second application of the cleanser composition the skin;
e) rinsing the second application of the cleaner composition from the skin;
f) applying an effective amount of the serum composition, the toner composition and an effective amount of the night moisturizer composition to the skin, wherein steps a-c and d-f are separated by about eight to about sixteen hours.
